(19) United States
(12) Patent Application Publication
(10) Pub. No.: US 2010/0204119 A1
(43) Pub. Date: Aug. 12, 2010

(54) 4-AMINO-5-OXO-7,8-DIHYDROPYRIMIDO[5,4-F][1,4]OXAZEPIN-6(5H)-YL PHENYL DERIVATIVES

(75) Inventors: Gary E. Aspnes, Rockville, RI (US); Robert L. Dow, Groton, CT (US); Michael J. Munchhof, Salem, CT (US)

Correspondence Address: PFIZER INC.;PATENT DEPARTMENT, Bld 114 M/S 9114, EASTERN POINT ROAD, GROTON, CT, 06340, US

(73) Assignee: Pfizer Inc

(21) Appl. No.: 12/697,658

(22) Filed: Feb. 1, 2010

Related U.S. Application Data
(60) Provisional application No. 61/149,056, filed on Feb. 2, 2009.
(60) Provisional application No. 61/285,380, filed on Dec. 10, 2009.

(51) Int. Cl. A61K 38/16; C07D 498/04; A61K 31/553; A61P 3/10; A61P 3/04; A61K 31/56

(52) U.S. Cl. 514/12; 540/490; 514/211.05; 514/171

(57) ABSTRACT

The invention provides compounds of Formula (I),

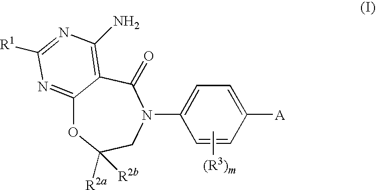

wherein R¹, R^{2a}, R^{2b}, R³, m and A are as defined herein, as well as compositions thereof and methods for treating a disease, condition or disorder that is modulated by the inhibition of the diacylglycerol O-acyltransferase 1 (DGAT-1) enzyme by administering the compounds of the present invention and/or compositions thereof.

This application claims priority from U.S. Provisional Application No. 61/149,056, filed on Feb. 2, 2009 and U.S. Provisional Application No. 61/285,380, filed on Dec. 10, 2009.

### FIELD OF THE INVENTION

The present invention relates to 4-amino-5-oxo-7,8-dihydropyrimido[5,4-f][1,4]oxazepin-6(5H)-yl)phenyl derivatives, as well as pharmaceutical compositions and uses thereof.

### BACKGROUND

It is estimated that somewhere between 34 and 61 million people in the US are obese and, in much of the developing world, incidence is increasing by about 1% per year. Obesity increases the likelihood of death from all causes by 20%, and more specifically, death from coronary artery disease and stroke are increased by 25% and 10%, respectively. Key priorities of anti-obesity treatments are to reduce food intake and/or hyperlipidemia. Since the latter has been suggested to provoke insulin resistance, molecules developed to prevent the accumulation of triglyceride would not only reduce obesity but they would also have the additional effect of reducing insulin resistance, a primary factor contributing to the development of diabetes. The therapeutic activity of leptin agonists has come under scrutiny through their potential to reduce food intake and, also, to reverse insulin resistance; however, their potential may be compromised by leptin-resistance, a characteristic of obesity. Acyl coenzyme A:diacylglycerol acyltransferase 1 (DGAT-1) is one of two known DGAT enzymes that catalyze the final step in mammalian triglyceride synthesis and an enzyme that is tightly implicated in both the development of obesity and insulin resistance. DGAT-1 deficient mice are resistant to diet-induced obesity through a mechanism involving increased energy expenditure. US researchers have now shown that these mice have decreased levels of tissue triglycerides, as well as increased sensitivity to insulin and to leptin. Importantly, DGAT-1 deficiency protects against insulin resistance and obesity in agouti yellow mice, a model of severe leptin resistance. Thus, DGAT-1 may represent a useful target for the treatment of insulin and leptin resistance and hence human obesity and diabetes. Chen, H. C., et al., J Clin Invest, 109(8), 1049-55 (2002).

Although studies show that DGAT-1 inhibition is useful for treating obesity and diabetes, there remains a need for DGAT-1 inhibitors that have efficacy for the treatment of metabolic disorders (e.g., obesity, Type 2 diabetes, and insulin resistance syndrome (also referred to as “metabolic syndrome”)).

### SUMMARY

The invention includes compounds of Formula (I)

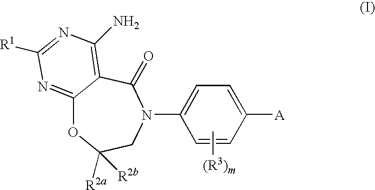

wherein

R¹ is hydrogen, (C₁-C₄)alkyl, (C₁-C₄)perfluoroalkyl, (C₁-C₄)perfluoroalkoxy, or (C₁-C₄)alkoxy;

R^{2a} and R^{2b}, taken separately, are each independently hydrogen, (C₁-C₄)alkyl, or (C₁-C₄)perfluoroalkyl, or R^{2a} and R^{2b}, taken together, are (C₃-C₆)cycloalkyl;

m is 0, 1 or 2;

R³ is halo, (C₁-C₄)alkyl, (C₃-C₈)cycloalkyl, (C₁-C₄)alkoxy, hydroxyl or CF₃, when m is 2, R³ can be the same or different and when m is 0, R³ is hydrogen;

A is a chemical moiety selected from the group consisting of
(i) (C₁-C₈)alkyl optionally substituted with one or two substituents selected from the group consisting of —N(R⁵)(R⁶), hydroxyl, (C₁-C₄)alkoxy, (C₁-C₄)haloalkyl, halo, cyano, —C(O)—OH, —C(O)—(C₁-C₄)alkoxy, and —C(O)—N(R⁵)(R⁶);
(ii) halo;
(iii) 3- to 5-membered carbocyclic ring optionally substituted with hydroxy, (C₁-C₄)alkoxy, cyano or 1 to 2 halo groups;
(iv) —C(O)—R⁴;
(v) a group of formula (Ia)

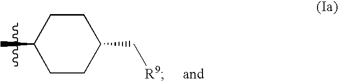

(vi) a group of formula (Ib)

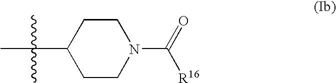

R⁴ is —OR⁵ or —N(R⁵)(R⁶);

R⁵ and R⁶ are each independently selected from H or (C₁-C₆)alkyl;

R⁹ is
(a) —(CH₂)ₚ—C(O)—N(R^{10a})(R^{10b}), where p is 0 or 1, R^{10a} is (C₁-C₆)alkyl-, or halo-substituted(C₁-C₃)alkyl-, and R^{10b} is —CH(CH₃)—R^{10c} or —(CH₂)_{q}R^{10c}, where q is 0, 1 or 2 and R^{10c} is (C₁-C₄)alkyl, —C(O)OH, —C(O)N((C₁-C₃)alkyl)₂, —C(O)NH(C₁-C₃)alkyl, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, (C₁-C₃)alkyl, (C₁-C₄)alkoxy, or cyano;
   or R^{10a} and R^{10b} taken together with the nitrogen to which they are attached form a 4- to 7-membered heterocycle optionally containing an additional heteroatom selected from oxygen, nitrogen or sulfur, where said heterocycle is optionally fused to a 5- to 6-membered heteroaryl containing 1 to 3 heteroatoms each independently selected from O, N or S, wherein said heterocycle and said fused heterocycle are optionally substituted with 1 to 3 substituents selected from hydroxyl, cyano, halo, (C₁-C₃)alkoxy-, (C₁-C₃)alkyl-, hydroxy(C₁-C₆)alkyl-, (C₁-C₃)alkoxy(C₁-C₃)alkyl-, CH₃C(O)NH—, CH₃C(O)—, or oxo;
(b) —(CH₂)ᵣR¹¹, where r is 0, 1 or 2 and R¹¹ is a chemical moiety selected from the group consisting of 1,3-thiazol-4-yl, 1,2,4-oxadiazol-5-yl, 1,3,4-oxadiazol-2-yl, 1,2,4-triazol-3-yl, 1,2,5-triazol-3-yl, or 1,3,4-thiadazol-2-yl; wherein said chemical moiety is optionally substituted with 1 to 3 (C₁-C₃)alkyl groups;
(c) —(CH₂)ₛ—C(OH)(R¹²)(R¹³), where s is 0, 1, or 2 and R¹² and R¹³ are each independently a H or (C₁-C₃)alkyl; or
(d) —(CH₂)ₜ—C(NH₂)(R¹⁴)(R¹⁵), where t is 0, 1, or 2 and R¹⁴ and R¹⁵ are each independently a H or (C₁-C₃)alkyl; and

R¹⁶ is (C₁-C₆)alkyl optionally substituted with hydroxyl, (C₁-C₃)alkoxy, (C₁-C₃)alkyl-SO₂—, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, or (C₁-C₃)alkyl;

or a pharmaceutically acceptable salt thereof.

The invention also includes compounds of Formula (I*)

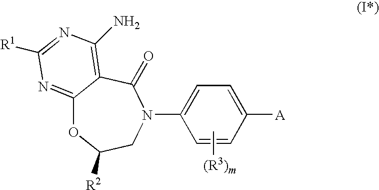

wherein, R¹ is hydrogen, (C₁-C₃)alkyl, methoxy or halo-substituted (C₁-C₃)alkyl (preferably, R¹ is hydrogen, methyl, —CF₃, or methoxy, more preferably, R¹ is hydrogen or methoxy); R² is hydrogen or methyl; m is 0, 1 or 2 (preferably, m is 0 or 1, more preferably, m is 0); R³ is halo, methyl, methoxy, or CF₃, when m is 2, R³ can be the same or different; A is a chemical moiety selected from the group consisting of
(i) (C₁-C₆)alkyl;
(ii) 3- to 5-membered carbocyclic ring optionally substituted with hydroxy, (C₁-C₄)alkoxy, cyano or 1 to 2 halo groups;
(iii) —C(CH₃)₂—R⁴, where R⁴ is cyano, hydroxyl, —C(O)NH₂, —C(O)—O(C₁-C₃)alkyl, —CH₂OH, or fluoro;
(iv) —C(O)O(C₁-C₃)alkyl;
(v) —C(O)—N(R⁵)(R⁶), where R⁵ and R⁶ are each independently selected from H or (C₁-C₃)alkyl; or
(vi) —(CH₂)ₙ—C(OH)(R⁷)(R⁸), where n is 0 or 1 and R⁷ and R⁸ are each independently a H, (C₁-C₃)alkyl, or —CF₃;
(vii) taken together with R³ on an adjacent carbon to form a 5- to 6-membered carbocyclic fused ring;
(viii) a group of formula (Ia)

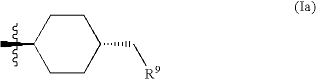

wherein R⁹ is
(a) —(CH₂)ₚ—C(O)—N(R^{10a})(R^{10b}), where p is 0 or 1, R^{10a} is (C₁-C₆)alkyl-, or halo-substituted(C₁-C₃)alkyl-, and R^{10b} is —CH(CH₃)—R^{10c} or —(CH₂)_{q}R^{10c}, where q is 0, 1 or 2 and R^{10c} is (C₁-C₄)alkyl, —C(O)OH, —C(O)N((C₁-C₃)alkyl)₂, —C(O)NH(C₁-C₃)alkyl, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, (C₁-C₃)alkyl, (C₁-C₄)alkoxy, or cyano;
   or R^{10a} and R^{10b} taken together with the nitrogen to which they are attached form a 4- to 7-membered heterocycle optionally containing an additional heteroatom selected from oxygen, nitrogen or sulfur, where said heterocycle is optionally fused to a 5- to 6-membered heteroaryl containing 1 to 3 heteroatoms each independently selected from O, N or S, wherein said heterocycle and said fused heterocycle are optionally substituted with 1 to 3 substituents selected from hydroxyl, cyano, halo, (C₁-C₃)alkoxy-, (C₁-C₃)alkyl-, hydroxy(C₁-C₆)alkyl-, (C₁-C₃)alkoxy(C₁-C₃)alkyl-, CH₃C(O)NH—, CH₃C(O)—, or oxo;
(b) —(CH₂)ᵣR¹¹, where r is 0, 1 or 2 and R¹¹ is a chemical moiety selected from the group consisting of 1,3-thiazol-4-yl, 1,2,4-oxadiazol-5-yl, 1,3,4-oxadiazol-2-yl, 1,2,4-triazol-3-yl, 1,2,5-triazol-3-yl, or 1,3,4-thiadazol-2-yl; wherein said chemical moiety is optionally substituted with 1 to 3 (C₁-C₃)alkyl groups;
(c) —(CH₂)ₛ—C(OH)(R¹²)(R¹³), where s is 0, 1, or 2 and R¹² and R¹³ are each independently a H or (C₁-C₃)alkyl; or
(d) —(CH₂)ₜ—C(NH₂)(R¹⁴)(R¹⁵), where t is 0, 1, or 2 and R¹⁴ and R¹⁵ are each independently a H or (C₁-C₃)alkyl; and
(ix) a group of formula (Ib)

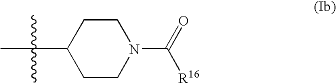

wherein R¹⁶ is
(a) —CH(CH₃)—R¹⁷ or —(CH₂)ᵥR¹⁷, where v is 0, 1 or 2 and R¹⁷ is hydrogen, (C₁-C₃)alkyl, (C₁-C₃)alkoxy, (C₁-C₃)alkyl-SO₂—, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, or (C₁-C₃)alkyl; or
(b) —(CH₂)_{w}—C(OH)(R¹⁸)(R¹⁹), where w is 0 or 1 and R¹⁸ and R¹⁹ are each independently a H or (C₁-C₃)alkyl;
   or a pharmaceutically acceptable salt thereof.

In one preferred embodiment, A is a chemical moiety selected from the group consisting of
(i) (C₁-C₆)alkyl;
(ii) 3- to 5-membered carbocyclic ring optionally substituted with hydroxy, (C₁-C₄)alkoxy, or 1 to 2 halo groups;
(iii) —C(CH₃)₂—R⁴, where R⁴ is cyano, hydroxyl, —C(O)NH₂, —C(O)—O(C₁-C₃)alkyl, —CH₂OH, or fluoro;
(iv) —C(O)O(C₁-C₃)alkyl;
(v) —C(O)—N(R⁵)(R⁶), where R⁵ and R⁶ are each independently selected from H or (C₁-C₃)alkyl; or
(vi) —(CH₂)ₙ—C(OH)(R⁷)(R⁸), where n is 0 or 1 and R⁷ and R⁸ are each independently a H, (C₁-C₃)alkyl, or —CF₃; and
(vii) taken together with R³ on an adjacent carbon to form a 5- to 6-membered carbocyclic fused ring;
   or a pharmaceutically acceptable salt thereof.

Another aspect of the invention is a pharmaceutical composition that comprises (1) a compound of the invention, and (2) a pharmaceutically acceptable excipient, diluent, or carrier. The composition may comprise a therapeutically effective amount of a compound of the invention. The composition may also contain at least one additional pharmaceutical agent. Such agents include anti-obesity agents and/or anti-diabetic agents.

In yet another aspect of the invention, a method for treating a disease, disorder, or condition modulated by DGAT-1 inhibition in animals is provided that includes the step of administering to an animal, such as a human, in need of such treatment a therapeutically effective amount of a compound of the invention (or a pharmaceutical composition thereof). Diseases, conditions, and/or disorders mediated by DGAT-1 inhibition include, e.g., obesity (including weight control or weight maintenance), Type 2 diabetes, diabetic nephropathy, insulin resistance syndrome, hyperglycemia, hyperinsulinemia, hyperlipidemia, impaired glucose tolerance, hypertension, and reducing the level of blood glucose.

Compounds of the invention may be administered in combination with other pharmaceutical agents (in particular, anti-obesity and anti-diabetic agents described herein below). The combination therapy may be administered as (a) a single pharmaceutical composition which comprises a compound of the invention, at least one additional pharmaceutical agent described herein and a pharmaceutically acceptable excipient, diluent, or carrier; or (b) two separate pharmaceutical compositions comprising (i) a first composition comprising a compound of the invention and a pharmaceutically acceptable excipient, diluent, or carrier, and (ii) a second composition comprising at least one additional pharmaceutical agent described herein and a pharmaceutically acceptable excipient, diluent, or carrier. The pharmaceutical compositions may be administered simultaneously or sequentially and in any order.

It is to be understood that both the foregoing summary and the following detailed description and attendant claims are exemplary and explanatory only and are not restrictive of the invention, as claimed.

### DETAILED DESCRIPTION

The invention may be understood even more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. The plural and singular should be treated as interchangeable, other than the indication of number.

The headings within this document are only being utilized to expedite its review by the reader. They should not be construed as limiting the invention or claims in any manner.

In this specification and in the claims that follow, reference will be made to a number of terms that shall be defined to have the following meanings:

As used herein in the specification, “a” or “an” may mean one or more. As used herein in the claim(s), when used in conjunction with the word “comprising”, the words “a” or “an” may mean one or more than one. As used herein “another” may mean at least a second or more.

The term “about” refers to a relative term denoting an approximation of plus or minus 10% of the nominal value it refers, in one embodiment, to plus or minus 5%, in another embodiment, to plus or minus 2%. For the field of this disclosure, this level of approximation is appropriate unless the value is specifically stated require a tighter range.

As used herein, the term “alkyl” refers to a hydrocarbon radical of the general formula CₙH₂ₙ₊₁. The alkane radical may be straight or branched. For example, the term “(C₁-C₆)alkyl” refers to a monovalent, straight, or branched aliphatic group containing 1 to 6 carbon atoms (e.g., methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like). Similarly, the alkyl portion (i.e., alkyl moiety) of an alkoxy group has the same definition as above. “Halo-substituted alkyl” or “halo-substituted alkoxy” refers to an alkyl or alkoxy group substituted with one or more halogen atoms (e.g., fluoromethyl, difluoromethyl, trifluoromethyl, perfluoroethyl, 1,1-difluoroethyl and the like).

The term “cycloalkyl” refers to nonaromatic rings that are fully hydrogenated and may exist as a single ring, bicyclic ring or a spiral ring. Unless specified otherwise, the carbocyclic ring is generally a 3- to 6-membered ring. For example, cycloalkyl include groups such as cyclopropyl, cyclobutyl, cyclopentyl, cyclohexyl, cyclohexenyl, and the like.

“Halogen” or “halo” refers to refers to a chlorine, fluorine, iodine, or bromine atom.

The phrase “therapeutically effective amount” means an amount of a compound of the invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” refers to humans (male or female), companion animals (e.g., dogs, cats and horses), food-source animals, zoo animals, marine animals, birds and other similar animal species. “Edible animals” refers to food-source animals such as cows, pigs, sheep and poultry.

The phrase “pharmaceutically acceptable” indicates that the substance or composition must be compatible chemically and/or toxicologically, with the other ingredients comprising a formulation, and/or the mammal being treated therewith.

The terms “treating”, “treat”, or “treatment” embrace both preventative, i.e., prophylactic, and palliative treatment.

The terms “modulated” or “modulating”, or “modulate(s)”, as used herein, unless otherwise indicated, refers to the inhibition of the diacylglycerol O-acyltransferase 1 (DGAT-1) enzyme with compounds of the invention.

The terms “mediated” or “mediating” or “mediate(s)”, as used herein, unless otherwise indicated, refers to the treatment or prevention the particular disease, condition, or disorder, (ii) attenuation, amelioration, or elimination of one or more symptoms of the particular disease, condition, or disorder, or (iii) prevention or delay of the onset of one or more symptoms of the particular disease, condition, or disorder described herein, by inhibiting the DGAT-1 enzyme.

The terms “compounds (or compound) of the present application (or invention)” or simply “compounds” or “compound” (unless specifically identified otherwise) refer to compounds described herein and pharmaceutically acceptable salts thereof, encompassed within this application, such as compounds encompassed within general formulas and intermediates of the compounds as well as salts, all stereoisomers (including diastereoisomers and enantiomers), tautomers, conformational isomers, and isotopically labeled compounds. Hydrates and solvates of the compounds of the invention are considered to be part of the invention, wherein the compound is in association with water or solvent, respectively.

The term “salt” and “pharmaceutically acceptable salt” refers to inorganic and organic salts of a compound. These salts can be prepared in situ during the final isolation and purification of a compound, or by separately reacting the present compound with a suitable organic or inorganic acid or base and isolating the salt thus formed. Representative salts include the hydrobromide, hydrochloride, hydroiodide, sulfate, bisulfate, nitrate, acetate, trifluoroacetate, oxalate, besylate, palmitate, pamoate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. See, e.g., Berge, et al., J. Pharm. Sci., 66, 1-19 (1977).

In one embodiment of the invention, A is a chemical moiety selected from the group consisting of
(i) (C₁-C₆)alkyl;
(ii) 3- to 5-membered carbocyclic ring optionally substituted with hydroxy, (C₁-C₄)alkoxy, cyano or 1 to 2 halo groups;
(iii) —C(CH₃)₂—R⁴, where R⁴ is cyano, hydroxyl, —C(O)NH₂, —C(O)—O(C₁-C₃)alkyl, —CH₂OH, or fluoro;
(iv) —C(O)O(C₁-C₃)alkyl;
(v) —C(O)—N(R⁵)(R⁶), where R⁵ and R⁶ are each independently selected from H or (C₁-C₃)alkyl;
(vi) —(CH₂)ₙ—C(OH)(R⁷)(R⁸), where n is 0 or 1 and R⁷ and R⁸ are each independently a H, (C₁-C₃)alkyl, or —CF₃; and
(vii) taken together with R³ on an adjacent carbon to form a 5- to 6-membered carbocyclic fused ring;
   or a pharmaceutically acceptable salt thereof.

In another embodiment of the invention,
R¹ is hydrogen or methoxy;
R² is methyl or hydrogen;
m is 0, or 1 when R³ and A are taken together to form a 5- to 6-membered carbocyclic fused ring;
A is
   (i) (C₁-C₄)alkyl;
   (ii) 3 to 4-membered carbocyclic ring optionally substituted with hydroxyl, methoxy, or 1 to 2 fluoro groups; or
   (iii) taken together with R³ on an adjacent carbon to form a 5- to 6-membered carbocyclic fused ring;
      or a pharmaceutically acceptable salt thereof.

In another embodiment of the invention, the compound has a Formula (II)

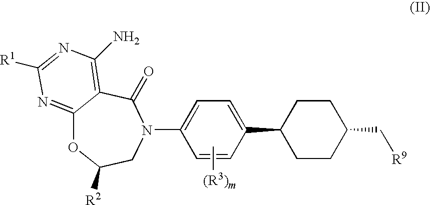

wherein
R¹ is hydrogen, (C₁-C₃)alkyl, methoxy or halo-substituted (C₁-C₃)alkyl;
R² is hydrogen or methyl;
m is 0, 1 or 2;
R³ is halo, methyl, methoxy, or CF₃, when m is 2, R³ can be the same or different;
R⁹ is selected from the group consisting of
   —(CH₂)ₚ—C(O)—N(R^{10a})(R^{10b}), where p is 0 or 1, R^{10a} is (C₁-C₆)alkyl-, or halo-substituted(C₁-C₃)alkyl-, and R^{10b} is —CH(CH₃)—R^{10c} or —(CH₂)_{q}R^{10c}, where q is 0, 1 or 2 and R^{10c} is (C₁-C₄)alkyl, —C(O)OH, —C(O)N((C₁-C₃)alkyl)₂, —C(O)NH(C₁-C₃)alkyl, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, (C₁-C₃)alkyl, (C₁-C₄)alkoxy, or cyano;
      or R^{10a} and R^{10b} taken together with the nitrogen to which they are attached form a 4- to 7-membered heterocycle optionally containing an additional heteroatom selected from oxygen, nitrogen or sulfur, where said heterocycle is optionally fused to a 5- to 6-membered heteroaryl containing 1 to 3 heteroatoms each independently selected from O, N or S, wherein said heterocycle and said fused heterocycle are optionally substituted with 1 to 3 substituents selected from hydroxyl, cyano, halo, (C₁-C₃)alkoxy-, (C₁-C₃)alkyl-, hydroxy(C₁-C₆)alkyl-, (C₁-C₃)alkoxy(C₁-C₃)alkyl-, CH₃C(O)NH—, CH₃C(O)—, or oxo;
   (ii) —(CH₂)ᵣR¹¹, where r is 0, 1 or 2 and R¹¹ is a chemical moiety selected from the group consisting of 1,3-thiazol-4-yl, 1,2,4-oxadiazol-5-yl, 1,2,4-triazol-3-yl, 1,2,5-triazol-3-yl, or 1,3,4-thiadazol-2-yl; wherein said chemical moiety is optionally substituted with 1 to 3 (C₁-C₃)alkyl groups;
   (iii) —(CH₂)ₛ—C(OH)(R¹²)(R¹³), where s is 0, 1, or 2 and R¹² and R¹³ are each independently a H or (C₁-C₃)alkyl; and
   (iv) —(CH₂)ₜ—C(NH₂)(R¹⁴)(R¹⁵), where t is 0, 1, or 2 and R¹⁴ and R¹⁵ are each independently a H or (C₁-C₃)alkyl;
      or a pharmaceutically acceptable salt thereof.

In another embodiment of the invention, R¹ is hydrogen; R² is methyl or hydrogen; m is 0; and R⁹ is
(i) —(CH₂)ₚ—C(O)—N(R^{10a})(R^{10b}), where p is 0, R^{10a} is (C₁-C₆)alkyl- and R^{10b} is —(CH₂)_{q}R^{10c}, where q is 1 and R^{10c} is phenyl, wherein said phenyl is optionally substituted with 1 to 3 substituents each independently selected from halo;
   or R^{10a} and R^{10b} taken together with the nitrogen to which they are attached form a 4- to 7-membered heterocycle optionally containing an additional heteroatom selected from oxygen or nitrogen, wherein said heterocycle is optionally substituted with 1 to 3 substituents selected from (C₁-C₃)alkyl-, or hydroxy(C₁-C₆)alkyl-;
(ii) —(CH₂)ᵣR¹¹, where r is 1 and R¹¹ is 1,2,4-oxadiazol-5-yl, wherein said 1,2,4-oxadiazol-5-yl is optionally substituted with 1 to 3 (C₁-C₃)alkyl groups; or
(iii) —(CH₂)ₛ—C(OH)(R¹²)(R¹³), where s is 1, or 2 and R¹² and R¹³ are each independently a H or (C₁-C₃)alkyl; or
   or a pharmaceutically acceptable salt thereof.

In another embodiment of the invention, the compound has a Formula (III)

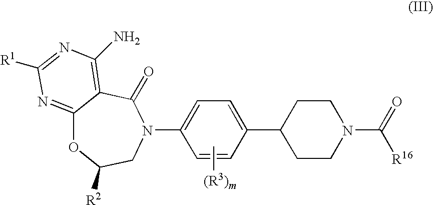

wherein
R¹ is hydrogen, (C₁-C₃)alkyl, methoxy, or halo-substituted (C₁-C₃)alkyl;
R² is hydrogen or methyl;
m is 0, 1 or 2;
R³ is halo, methyl, methoxy, or CF₃, when m is 2, R³ can be the same or different;
R¹⁶ is
   (i) —CH(CH₃)—R¹⁷ or —(CH₂)ᵥR¹⁷, where v is 0, 1 or 2 and R¹⁷ is hydrogen, (C₁-C₃)alkyl, (C₁-C₃)alkoxy, (C₁-C₃)alkyl-SO₂—, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, or (C₁-C₃)alkyl; or
   (ii) —(CH₂)_{w}—C(OH)(R¹⁸)(R¹⁹), where w is 0 or 1 and R¹⁸ and R¹⁹ are each independently a H or (C₁-C₃)alkyl;
      or a pharmaceutically acceptable thereof.

In another embodiment of the invention, R¹ is hydrogen; R² is methyl or hydrogen;

m is 0; R¹⁶ is —(CH₂)ᵥR¹⁷, where v is 0, 1 or 2 and R¹⁷ is (C₁-C₃)alkyl, a 5- to 6-membered cycloalkyl, phenyl, or a 5- to 6-membered heteroaryl containing 1 to 3 heteroatoms each independently selected from oxygen, or nitrogen;

or a pharmaceutically acceptable thereof.

Another embodiment of the invention includes a pharmaceutical composition comprising (i) a compound of any one of the preceding claims; and (ii) a pharmaceutically acceptable excipient, diluent, or carrier.

In another embodiment, the compound or pharmaceutically acceptable salt thereof is present in a therapeutically effective amount.

In yet another embodiment, the composition further comprises at least one additional pharmaceutical agent selected from the group consisting of an anti-obesity agent and an anti-diabetic agent.

In another embodiment, said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine and said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

Another embodiment of the invention includes a method for treating or delaying the progression or onset of Type 2 diabetes and diabetes-related disorders in animals comprising the step of administering to an animal in need of such treatment a therapeutically effective amount of a compound described herein.

In another embodiment of the invention, the method for treating or delaying the progression or onset of Type 2 diabetes and diabetes-related disorders in animals comprises the step of administering to an animal in need of such treatment a pharmaceutical composition described herein.

In another embodiment of the invention, the method for treating a disease, condition or disorder modulated by the inhibition of DGAT-1 in animals comprises the step of administering to an animal in need of such treatment two separate pharmaceutical compositions comprising
(i) a first composition comprising a compound described herein, and a pharmaceutically acceptable excipient, diluent, or carrier; and
(ii) a second composition comprising at least one additional pharmaceutical agent selected from the group consisting of an anti-obesity agent and an anti-diabetic agent, and a pharmaceutically acceptable excipient, diluent, or carrier;
   wherein said disease, condition or disorder modulated by the inhibition of DGAT-1 is selected from the group consisting of obesity, obesity-related disorders, Type 2 diabetes, and diabetes-related disorders.

In one embodiment, said first composition and said second composition are administered simultaneously. In another embodiment, said first composition and said second

Yet another embodiment includes the use of a compound of the invention or a pharmaceutically acceptable salt thereof in the manufacture of a medicament for treating a disease, condition or disorder that is modulated by the inhibition of DGAT-1.

The invention also includes solvates and hydrates of the compounds of the invention. The term “solvate” refers to a molecular complex of a compound of this invention (including pharmaceutically acceptable salts thereof) with one or more solvent molecules. Such solvent molecules are those commonly used in the pharmaceutical art, which are known to be innocuous to the recipient, e.g., water, ethanol, ethylene glycol, and the like, The term “hydrate” refers to the complex where the solvent molecule is water. The solvates and/or hydrates may exist in crystalline form. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like.

The compounds of the invention may contain asymmetric or chiral centers, and, therefore, exist in different stereoisomeric forms. Unless specified otherwise, it is intended that all stereoisomeric forms of the compounds of the invention as well as mixtures thereof, including racemic mixtures, form part of the invention. In addition, the invention embraces all geometric and positional isomers. For example, if a compound of the invention incorporates a double bond or a fused ring, both the cis- and trans-forms, as well as mixtures, are embraced within the scope of the invention.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the invention may be atropisomers (e.g., substituted biaryls) and are considered as part of this invention. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

It is also possible that the intermediates and compounds of the invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

Certain compounds of the invention may exist in different stable conformational forms which may be separable. Torsional asymmetry due to restricted rotation about an asymmetric single bond, for example, because of steric hindrance or ring strain, may permit separation of different conformers.

The invention also embraces isotopically-labeled compounds of the invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the invention (e.g., those labeled with ³H and ¹⁴C) are useful in compound and/or substrate tissue distribution assays. Tritiated (i.e., ³H) and carbon-14 (i.e., ¹⁴C) isotopes may be used for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be used in some circumstances. Positron emitting isotopes such as ¹⁵O, ¹³N, ¹¹C, and ¹⁸F are useful for positron emission tomography (PET) studies to examine substrate occupancy. Isotopically labeled compounds of the invention can generally be prepared by following procedures analogous to those disclosed in the Schemes and/or in the Examples herein below, by substituting an isotopically labeled reagent for a non-isotopically labeled reagent.

Certain compounds of the invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the invention followed by gradual or fast cooling. The presence of polymorphs may be determined by solid probe NMR spectroscopy, IR spectroscopy, differential scanning calorimetry, powder X-ray diffraction or such other techniques.

In general, compounds of this invention may be prepared by methods that include processes known in the chemical arts, particularly in light of the description contained herein in combination with the knowledge of the skilled artisan. Although other reagents, starting materials, intermediate compounds or methods can be used in practice or testing, generalized methods for the preparation of the compounds of the invention are illustrated by the following descriptions, Preparations, and reaction Schemes. Other preparation methods are described in the experimental section. The methods disclosed herein, including those outlined in the Schemes, Preparations, and Examples are for intended for illustrative purposes and are not to be construed in any manner as limitations thereon.

The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

Compounds of the invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

In the preparation of compounds of the invention, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

Scheme I outlines the general procedures one could use to provide compounds of the invention having Formula (I) and (I*).

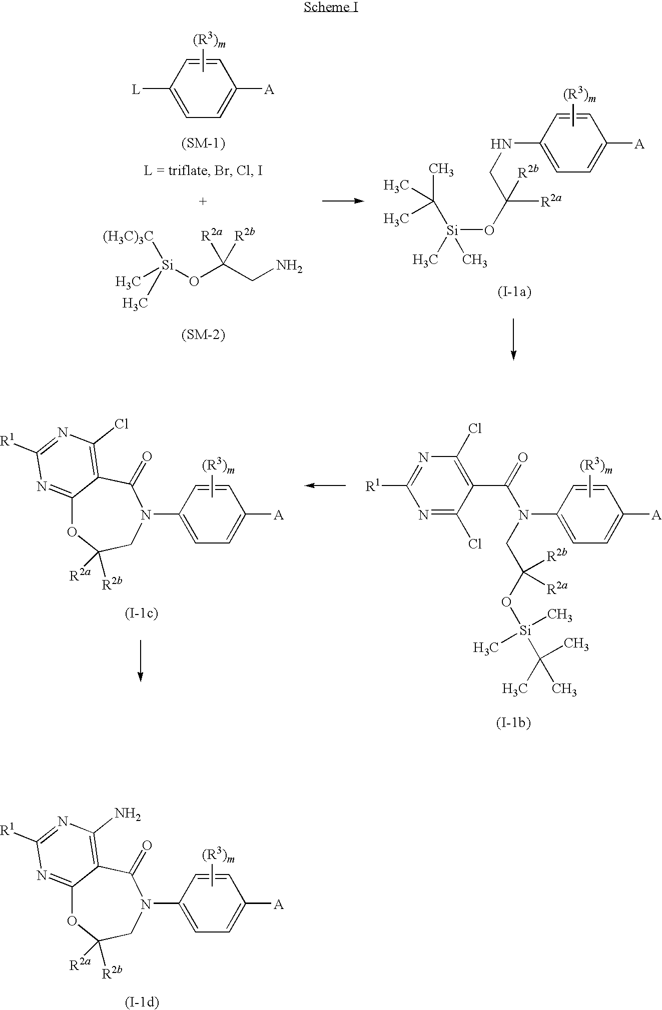

Scheme I has been modified.

The desired starting material (SM-1) can be prepared as described in the intermediate section. The 2-{[tert-butyl(dimethyl)silyl]-oxy}ethanamine (SM-2) can be prepared by various methods including those disclosed in JACS, 129(37), 11408-11420 (2007): Organic Letters, 9(1), 101-104 (2007); or Bioorganic & Medicinal Chemistry, 13(11), 3821-3839 (2005). The tert-butyl(dimethyl)silyl group provides a convenient protecting group for the hydroxyl moiety in subsequent reactions. The two starting materials can be coupled together at elevated temperatures (e.g., about 80° C. to about 130° C.) in the presence of a palladium (or copper) catalyst, a weak base (e.g., cesium carbonate), and 2-dicyclohexyl phosphino-2′,4′,6′-triisopropylbiphenyl (X-PHOS) in an inert environment to form intermediate (I-1a). The desired 4,6-dichloropyrimidine carbonyl moiety is then added to intermediate (I-1a) via an acylation onto the secondary amino group using procedures well known to those of skill in the art (e.g., addition of 4,6-dichloropyrimidine-5-carbonyl chloride in the presence of a mild base, such as triethylamine or pyridine) to form intermediate (I-1b). See, e.g., Tarasov, E., et al., Synlett (5), 625-626 (2005). The silyloxy protecting group can then be removed (e.g., treatment with HCl in a protic solvent, such as methanol). Once the protecting group is removed, then the cyclized lactam (I-1c) can be formed by treatment with a base (e.g., triethylamine or potassium carbonate) in an aprotic solvent at about 20° C. to about 120° C. Preferably, the cyclization is carried out with triethylamine in acetonitrile at a temperature from about 40° C. to about 120° C. Amination of lactam intermediate (I-1c) can be accomplished with ammonia in an aproptic or protic solvent at a temperature between about 0° C. to about 100° C. for about 4 to about 24 hours to form intermediate (I-1d).

Scheme II below describes how one can produce compounds of Formula (II) where R⁹ is —(CH₂)ₚ—C(O)—N(R^{10a})(R^{10b}).

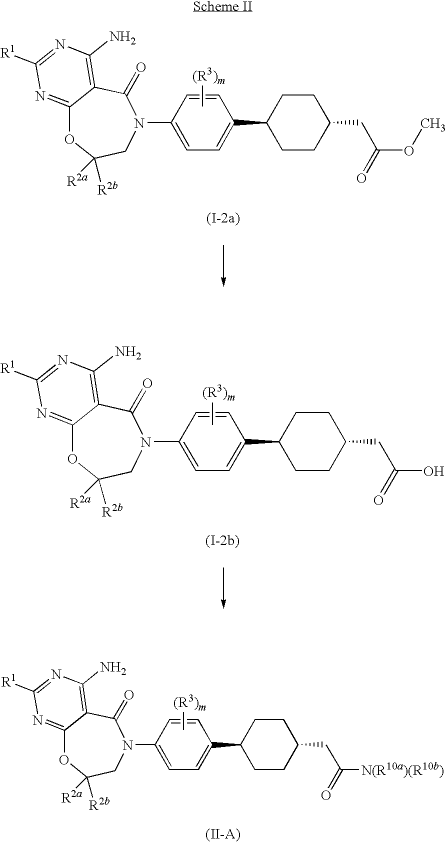

Scheme II has been modified.

The ester (I-1a) may be prepared using the procedures described above in Scheme I where the starting material (SM-1) is the desired trans-4-[4-[[trifluoromethyl)sulfonyl]oxy]phenyl]-cyclohexyl]acetate. The ester (I-1a) can be reacted with a variety of moieties to provide the acid (I-2b), such as treatment with acid or base in the presence of water. The acid (I-2b) can then be coupled with the desired amine (HN(R^{10a})R^{10b})) using conventional peptide coupling reactions to produce the amide (II-A). Alternatively, the ester (I-1a) can be directly condensed with the desired amine (HN(R^{10a})R^{10b})) to produce the amide (II-A).

Scheme III below describes how one could make compounds of Formula III.

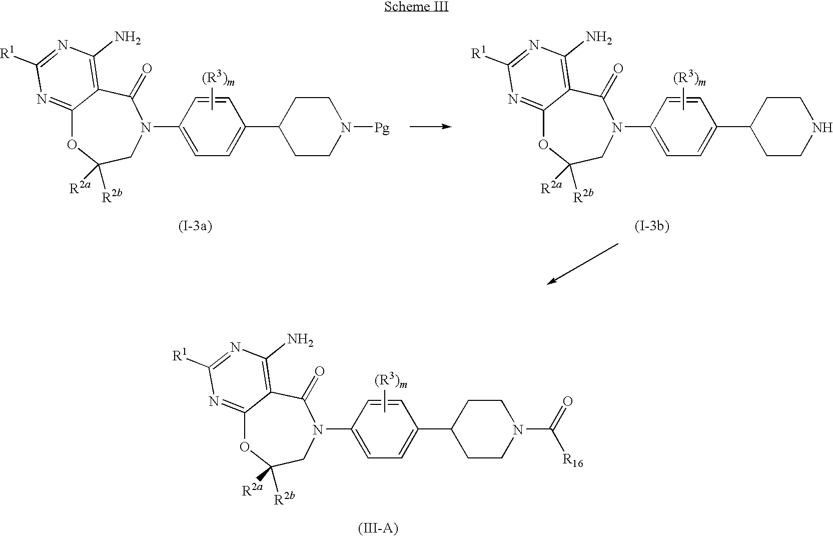

Scheme III has been modified.

Intermediate (I-3a) may be prepared using the procedures described in Scheme I above where the starting material (SM-1) is the desired amino-protected 4-[4-[[trifluoromethyl)sulfonyl]oxy]phenyl]-piperidine. The amino-protecting group may be removed using the procedures appropriate for the particular protecting group used. For example, when the protecting group (Pg) is a t-butoxycarbonyl, then the group may be removed by treatment with acid (e.g., trifluoroacetic or hydrochloric acid). The amino intermediate (I-3b) can then be condensed with the desired acid (R¹⁶CO₂H) utilizing standard amide coupling conditions to produce the N-acylated compound (III-A). Alternatively, amino intermediate (I-3b) can be reacted with the appropriate acid chloride (R¹⁶COCl) in the presence of a base (preferably, triethylamine) to provide the amide compound (III-A).

Compounds of the invention are useful for treating diseases, conditions and/or disorders modulated by the inhibition of the DGAT-1 enzyme; therefore, another embodiment of the invention is a pharmaceutical composition comprising a therapeutically effective amount of a compound of the invention and a pharmaceutically acceptable excipient, diluent or carrier. The compounds of the invention (including the compositions and processes used therein) may also be used in the manufacture of a medicament for the therapeutic applications described herein.

A typical formulation is prepared by mixing a compound of the invention and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the invention or pharmaceutical composition thereof) or aid in the manufacturing of the pharmaceutical product (i.e., medicament).

The formulations may be prepared using conventional dissolution and mixing procedures. For example, the bulk drug substance (i.e., compound of the invention or stabilized form of the compound (e.g., complex with a cyclodextrin derivative or other known complexation agent)) is dissolved in a suitable solvent in the presence of one or more of the excipients described above. The compound of the invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The invention further provides a method of treating diseases, conditions and/or disorders modulated by the inhibition of the DGAT-1 enzyme in an animal that includes administering to an animal in need of such treatment a therapeutically effective amount of a compound of the invention or a pharmaceutical composition comprising an effective amount of a compound of the invention and a pharmaceutically acceptable excipient, diluent, or carrier. The method is particularly useful for treating diseases, conditions and/or disorders that benefit from the inhibition of DGAT-1.

One aspect of the invention is the treatment of obesity, and obesity-related disorders (e.g., overweight, weight gain, or weight maintenance).

Obesity and overweight are generally defined by body mass index (BMI), which is correlated with total body fat and estimates the relative risk of disease. BMI is calculated by weight in kilograms divided by height in meters squared (kg/m²). Overweight is typically defined as a BMI of 25-29.9 kg/m², and obesity is typically defined as a BMI of 30 kg/m². See, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083 (1998).

Another aspect of the invention is for the treatment or delaying the progression or onset of diabetes or diabetes-related disorders including Type 1 (insulin-dependent diabetes mellitus, also referred to as “IDDM”) and Type 2 (noninsulin-dependent diabetes mellitus, also referred to as “NIDDM”) diabetes, impaired glucose tolerance, insulin resistance, hyperglycemia, and diabetic complications (such as atherosclerosis, coronary heart disease, stroke, peripheral vascular disease, nephropathy, hypertension, neuropathy, and retinopathy).

Yet another aspect of the invention is the treatment of diabetes- or obesity-related co-morbidities, such as metabolic syndrome. Metabolic syndrome includes diseases, conditions or disorders such as dyslipidemia, hypertension, insulin resistance, diabetes (e.g., Type 2 diabetes), weight gain, coronary artery disease and heart failure. For more detailed information on Metabolic Syndrome, see, e.g., Zimmet, P. Z., et al., “The Metabolic Syndrome Perhaps an Etiologic Mystery but Far From a Myth—Where Does the International Diabetes Federation Stand?,” Diabetes & Endocrinology, 7(2), (2005); and Alberti, K. G., et al., “The Metabolic Syndrome—A New Worldwide Definition,” Lancet, 366, 1059-62 (2005). Administration of the compounds of the invention may provide a statistically significant (p<0.05) reduction in at least one cardiovascular disease risk factor, such as lowering of plasma leptin, C-reactive protein (CRP) and/or cholesterol, as compared to a vehicle control containing no drug. The administration of compounds of the invention may also provide a statistically significant (p<0.05) reduction in glucose serum levels.

In yet another aspect of the invention, the condition treated is impaired glucose tolerance, hyperglycemia, diabetic complications such as sugar cataracts, diabetic neuropathy, diabetic nephropathy, diabetic retinopathy and diabetic cardiomyopathy, anorexia nervosa, bulimia, cachexia, hyperuricemia, hyperinsulinemia, hypercholesterolemia, hyperlipidemia, dyslipidemia, mixed dyslipidemia, hypertriglyceridemia, nonalcoholic fatty liver disease, atherosclerosis, arteriosclerosis, acute heart failure, congestive heart failure, coronary artery disease, cardiomyopathy, myocardial infarction, angina pectoris, hypertension, hypotension, stroke, ischemia, ischemic reperfusion injury, aneurysm, restenosis, vascular stenosis, solid tumors, skin cancer, melanoma, lymphoma, breast cancer, lung cancer, colorectal cancer, stomach cancer, esophageal cancer, pancreatic cancer, prostate cancer, kidney cancer, liver cancer, bladder cancer, cervical cancer, uterine cancer, testicular cancer and ovarian cancer.

The invention also relates to therapeutic methods for treating the above described conditions in a mammal, including a human, wherein a compound of this invention is administered as part of an appropriate dosage regimen designed to obtain the benefits of the therapy. The appropriate dosage regimen, the amount of each dose administered and the intervals between doses of the compound will depend upon the compound of this invention being used, the type of pharmaceutical compositions being used, the characteristics of the subject being treated and the severity of the conditions.

The invention also provides pharmaceutical compositions which comprise a therapeutically effective amount of a compound, or a pharmaceutically acceptable salt thereof, in admixture with at least one pharmaceutically acceptable excipient. The compositions include those in a form adapted for oral, topical or parenteral use and can be used for the treatment of diabetes and related conditions as described above.

The composition can be formulated for administration by any route known in the art, such as subdermal, inhalation, oral, topical, parenteral, etc. The compositions may be in any form known in the art, including but not limited to tablets, capsules, powders, granules, lozenges, or liquid preparations, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% to about 99 by weight, of the active material, depending on the method of administration. Where the compositions comprise dosage units, each unit will contain, for example, from about 0.1 to 900 mg of the active ingredient, more typically from 1 mg to 250 mg, or 0.01 mg/kg/day to 30 mg/kg/day, such as 0.01 mg/kg/day to 5 mg/kg/day of active compound in single or divided doses.

Compounds of the invention can be formulated for administration in any convenient way for use in human or veterinary medicine, by analogy with other anti-diabetic agents. Such methods are known in the art and have been summarized above. For a more detailed discussion regarding the preparation of such formulations; the reader's attention is directed to Remington's Pharmaceutical Sciences, 21^{st} Edition, by University of the Sciences in Philadelphia.

It is also noted that the compounds of the invention can be used in sustained release, controlled release, and delayed release formulations, which forms are also well known to one of ordinary skill in the art.

The compounds of this invention may also be used in conjunction with other pharmaceutical agents for the treatment of the diseases, conditions and/or disorders described herein. Therefore, methods of treatment that include administering compounds of the invention in combination with other pharmaceutical agents are also provided. Suitable pharmaceutical agents that may be used in combination with the compounds of the invention include anti-obesity agents (including appetite suppressants), anti-diabetic agents, anti-hyperglycemic agents, lipid lowering agents, and anti-hypertensive agents.

Suitable anti-diabetic agents include an acetyl-CoA carboxylase-2 (ACC-2) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidase IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist and a glucokinase activator. Exemplary anti-diabetic agents are metformin and DPP-IV inhibitors (e.g., sitagliptin, vildagliptin, alogliptin and saxagliptin).

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆ (including analogs thereof), thyromimetic agents, dehydroepiandrosterone or an analog thereof, glucocorticoid agoniits or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Exemplary anti-obesity agents for use in the combination aspects of the invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆ (as used herein “PYY₃₋₃₆” includes analogs, such as pegylated PYY₃₋₃₆ e.g., those described in Wellesley, Mass.) using the sodium D line (λ=589 nm) at the indicated temperature and are reported as follows [α]_{D}^{temp}, concentration (c=g/100 ml), and solvent.

Column chromatography was performed with either Baker silica gel (40 μm; J. T. Baker, Phillipsburg, N.J.) or Silica Gel 50 (EM Sciences™, Gibbstown, N.J.) in glass columns or in Flash 40 Biotage™ columns (ISC, Inc., Shelton, Conn.) or Biotage™ SNAP cartridge KPsil or Redisep Rf silica (from Teledyne™ Isco™) under low nitrogen pressure.

### Starting Materials

Methyl [trans-4-[4-[[(trifluoromethyl)sulfonyl]oxy]phenyl]cyclohexyl]acetate was prepared as described for Compound 56 in U.S. Pat. No. 7,244,727, incorporated herein by reference.

2-{[Tert-butyl(dimethyl)silyl]oxy}ethanamine can be prepared by various methods including those disclosed in Journal of the American Chemical Society, 129(37), 11408-11420 (2007); Organic Letters, 9(1), 101-104 (2007); or Bioorganic & Medicinal Chemistry, 13(11), 3821-3839 (2005).

(R)-2-(tert-butyldimethylsilyloxy)propan-1-amine can be prepared by various methods including those disclosed in the Journal of Organic Chemistry, 72(20), 7726-7735 (2007).

### Preparation of Key Intermediates

### Preparation of Intermediate Methyl (trans-4-{4-[(2-{[tert-butyl(dimethyl)-silyl]oxy}ethyl)amino]-phenyl}-cyclohexyl)acetate (I-1a-1)

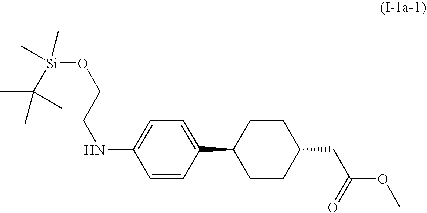

A mixture of methyl [trans-4-[4-[[(trifluoromethyl)-sulfonyl]oxy]phenyl]-cyclohexyl]acetate (10.1 g, 26.6 mmol), 2-{[tert-butyl(dimethyl)silyl]oxy}ethanamine (5.59 g, 31.9 mmol), cesium carbonate (8.65 g, 26.6 mmol), palladium acetate (0.60 g, 2.66 mmol) and X-PHOS (1.27 g, 2.66 mmol) in toluene (53 mL) under nitrogen was heated in a sealed tube at 120° C. for 16 hours. The reaction was cooled, diluted into EtOAc, washed with water (2×), saturated aqueous brine, dried over sodium sulfate and concentrated to afford a dark oil. Chromatography (330 g Biotage Snap Cartridge® silica gel column, 0-15% EtOAc:heptane) afforded methyl (trans-4-{4-[(2-{[tert-butyl(dimethyl)silyl]oxy}ethyl)-amino]phenyl}cyclohexyl)acetate (I-1a-1) as a light-yellow oil, 6.70 g.

¹H NMR (400 MHz, CDCl₃): δ 7.02, (d, 2H), 6.61 (d, 2H), 3.80 (m, 2H), 3.64 (s, 3H), 3.20 (m, 2H), 2.37 (m, 1H), 2.24 (m, 2H), 1.85 (m, 5H), 1.44 (m, 2H), 1.13 (m, 2H), 0.87 (s, 9H), 0.04 (s, 6H). m/z=406.4 (M+1).

### Preparation of Intermediate Methyl [trans-4-(4-{(2-{[tert-butyl(dimethyl)silyl]oxy}-ethyl)[(4,6-dichloropyrimidin-5-yl)carbonyl]amino}phenyl)cyclohexyl]acetate (I-1b-1)

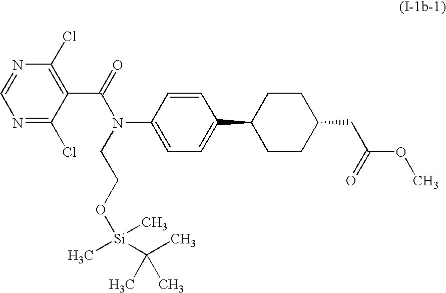

To a stirred, cooled (0° C.) solution of methyl (trans-4-{4-[(2-{[tert-butyl-(dimethyl)silyl]oxy}ethyl)amino]phenyl}cyclohexyl)acetate (I-1a-1: 9.7 g, 24.0 mmol), and triethylamine (3.53 mL, 25.3 mmol) in THF (60 mL) was added dropwise a solution of 4,6-dichloropyrimidine-5-carbonyl chloride (5.31 g, 25.1 mmol) in THF (20 mL). After 2 hours, the reaction was concentrated in vacuo, diluted into EtOAc, washed with water (3×), saturated aqueous brine, dried over sodium sulfate and concentrated in vacuo, to afford an oil (I-1b-1), which was carried on to the next step without further purification.

¹H NMR (400 MHz, CDCl₃): δ 8.57 (s, 1H), 7.35 (d, 2H), 7.03 (d, 2H), 4.00 (m, 2H), 3.87 (m, 2H), 3.63 (s, 3H), 2.37 (m, 1H), 2.22 (m, 2H), 1.82 (m, 5H), 1.36 (m, 2H), 1.11 (m, 2H), 0.83 (s, 9H), 0.02 (s, 6H). m/z=580.3 (M+1).

### Preparation of Intermediate Methyl (trans-4-{4-[[4,6-dichloropyrimidin-5-yl)carbonyl](2-hydroxyethyl)amino]phenyl}cyclohexyl)acetate (I-1c-1)

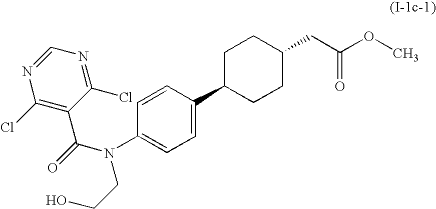

A solution of methyl [trans-4-(4-{(2-{[tert-butyl(dimethyl)silyl]oxy}ethyl)[(4,6-dichloropyrimidin-5-yl)carbonyl]amino}phenyl)cyclohexyl]acetate (I-1b-1: 14.0 g, 24.0 mmol) in a methanolic solution of HCl (3 mL of concentrated aqueous HCl in 97 mL of methanol) was stirred at room temperature for 30 minutes. Methanol was removed in vacuo, the residue was dissolved in EtOAc, washed with saturated aqueous sodium bicarbonate, saturated aqueous brine, dried over sodium sulfate and concentrated in vacuo to afford an oil (I-1c-1), which was carried on to the next step without further purification.

¹H NMR (400 MHz, CDCl₃): δ 8.59 (s, 1H), 7.32 (d, 2H), 7.04 (d, 2H), 4.08 (m, 2H), 3.92 (m, 2H), 3.63 (s, 3H), 2.38 (m, 1H), 2.23 (m, 2H), 1.82 (m, 5H), 1.39 (m, 2H), 1.11 (m, 2H). m/z=466.2 (M+1).

### Preparation of Intermediate Methyl {trans-4-[4-(4-chloro-5-oxo-7,8-dihydropyrimido[5,4-f][1,4]oxazepin-6-(5H)-yl)phenyl]cyclohexyl}acetate (I-1d-1)

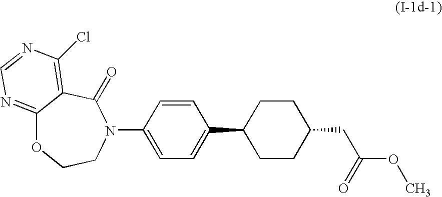

A slurry of methyl (trans-4-{4-[[4,6-dichloropyrimidin-5-yl)carbonyl](2-hydroxy-ethyl)amino]phenyl}cyclohexyl)acetate (I-1c-1: 4.78 g, 10.2 mmol, unpurified material) and triethylamine (4.15 g, 41 mmol) in acetonitrile was stirred at 80° C. for 6 hours. The reaction was cooled, concentrated in vacuo, diluted into EtOAc, washed with water (3×), saturated aqueous brine, dried over sodium sulfate and concentrated in vacuo to afford a yellow solid. This material was slurried in methanol (10 mL), filtered, the solids washed with methanol (2×3 mL) and dried in vacuo to afford the title compound (I-1d-1) as a yellow solid, 4.03 g.

¹H NMR (400 MHz, CDCl₃): δ 8.75 (s, 1H), 7.22 (s, 4H), 4.75 (m, 2H), 4.03 (m, 2H), 3.63 (s, 3H), 2.50 (m, 1H), 2.23 (m, 2H), 1.87 (m, 5H), 1.44 (m, 2H), 1.19 (m, 2H). m/z=430.3 (M+1).

### Preparation of Intermediate Methyl {trans-4-[4-(4-amino-5-oxo-7,8-dihydropyrimido[5,4-f][1,4]oxazepin-6-(5H)-yl)phenyl]cyclohexyl}acetate (I-1e-1)

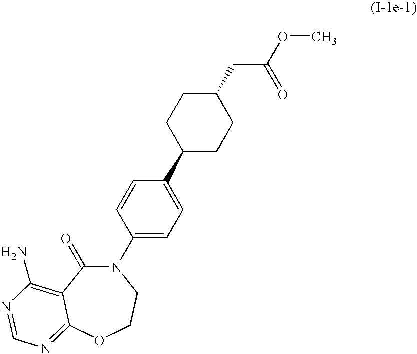

A solution of methyl {trans-4-[4-(4-chloro-5-oxo-7,8-dihydropyrimido[5,4-f][1,4]oxazepin-6-(5H)-yl)phenyl]cyclohexyl}acetate (I-1d-1: 5.29 g, 12.3 mmol) in 0.5M ammonia in p-dioxane (120 mL) was stirred at room temperature for 24 hours. The reaction mixture was concentrated in vacuo, diluted into EtOAc (1 L), washed with water, saturated aqueous brine, dried over sodium sulfate and concentrated in vacuo to afford the title compound (I-1e-1) as an off-white solid, 5.04 g.

¹H NMR (400 MHz, CDCl₃): δ8.22 (s, 1H), 8.16 (br s, 1H), 7.23 (d, 2H), 7.16 (d, 2H), 5.75 (br s, 1H), 4.63 (m, 2H), 3.98 (m, 2H), 3.64 (s, 3H), 2.44 (m, 1H), 2.21 (m, 2H), 1.81 (m, 5H), 1.42 (m, 2H), 1.10 (m, 2H). m/z=411.3 (M+1). IC₅₀ 34.5 nM (range 30-40 nM).

### Preparation of Intermediate {Trans-4-[4-(4-amino-5-oxo-7,8-dihydropyrimido[5,4-f][1,4]oxazepin-6(5H)-yl)phenyl]cyclohexyl}acetic acid (I-1f-1)

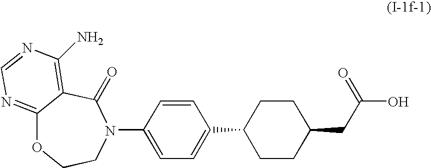

A stirred solution of methyl {trans-4-[4-(4-amino-5-oxo-7,8-dihydropyrimido[5,4-f][1,4]oxazepin-6-(5H)-yl)phenyl]cyclohexyl}acetate (I-1e-1: 5.05 g, 12.3 mmol) and 1N aqueous lithium hydroxide (36.9 mL) in p-dioxane (96 mL) and water (27 mL) was stirred at 50° C. for one hour. After cooling, the reaction solution was adjusted to pH ˜3.5 with 6N aqueous hydrochloric acid and the mixture was concentrated to near dryness. This residue was slurried in water (40 mL) for 1 hour, filtered, the solids washed with water (2×20 mL), ether (3×30 mL) and dried in vacuo to afford the title compound (I-1f-1) as an off-white solid, 4.58 g.

¹H NMR (400 MHz, DMSO-d₆): δ 8.12 (s, 1H), 7.58 (br s, 2H) 7.21 (s, 4H), 4.56 (m, 2H), 3.92 (m, 2H), 2.42 (m, 1H), 2.08 (m, 2H), 1.75 (m, 5H), 1.42 (q, 2H), 1.05 (q, 2H). m/z=397.3 (M+1). IC₆₀ 19.1 nM (range 5.2-63.6 nM).

### Preparation of Intermediate Methyl [trans-4-(4-{[(2R)-2-{[tert-butyl(dimethyl)silyl]oxy}propyl]-amino}phenyl)cyclohexyl]acetate (I-1a-2)

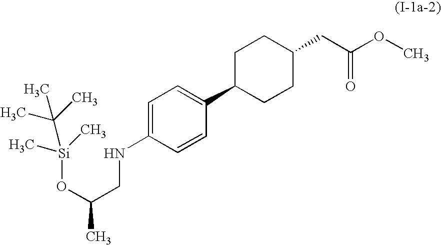

A mixture of methyl [trans-4-[4-[[trifluoromethyl)sulfonyl]oxy]phenyl]-cyclohexyl]acetate (5.00 g, 13.1 mmol), (R)-2-(tert-butyldimethylsilyloxy)propan-1-amine (2.99 g, 15.8 mmol), cesium carbonate (5.14 g, 15.8 mmol), palladium acetate (310 mg, 1.32 mmol) and X-PHOS (627 mg, 1.32 mmol) in toluene (100 mL) under nitrogen was heated in a sealed tube at 120° C. for 16 hours. The reaction was cooled, diluted into EtOAc (500 mL), washed with water (2×200 mL), saturated aqueous brine, dried over sodium sulfate and concentrated to afford a dark oil. Chromatography (120 g silica gel column, 3-15% EtOAc:heptane) afforded methyl [trans-4-(4-{[(2R)-2-{[tert-butyl(dimethyl)silyl)oxy}propyl]amino}phenyl)cyclohexyl]acetate (I-1a-2) as a light-yellow oil, 4.55 g (86%). m/z=420.1 (M+1).

### Preparation of Intermediate methyl [trans-4-(4-{[(2R)-2-{[tert-butyl(dimethyl)silyl]oxy}propyl][(4,6-dichloropyrimidin-5-yl)carbonyl]amino}-phenyl)cyclohexyl]acetate (I-1b-2)

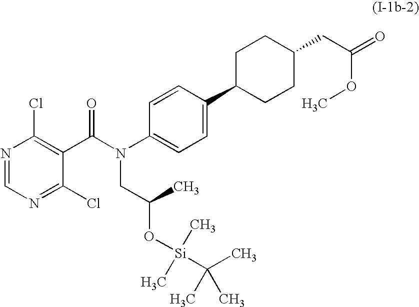

A mixture of 4,6-dichloropyrimidine-5-carbonyl chloride (2.27 g, 10.7 mmol), methyl [trans-4-(4-{[(2R)-2-{[tert-butyl(dimethyl)silyl]oxy}propyl]-amino}phenyl)cyclohexyl]acetate (I-1a-2: 4.50 g, 10.7 mmol) and triethylamine (2.24 mL, 16.1 mmol) in THF (150 mL) was stirred at room temperature under nitrogen for 14 hours. The reaction mixture was concentrated to remove THF. The residue was diluted with ethyl acetate (300 mL), washed with water (2×200 mL), dried over MgSO₄ and concentrated. The crude material was purified by a 120 g silica gel column eluted with 3-15% ethyl acetate in heptane to give methyl [trans-4-(4-{[(2R)-2-{[tert-butyl(dimethyl)silyl]oxy}propyl][(4,6-dichloropyrimidin-5-yl)carbonyl]amino}-phenyl)cyclohexyl]acetate (I-1b-2) as a colorless oil 4.01 g (63%).

m/z=594.2 (M+1). 1H NMR (400 MHz, chloroform-d) δ −0.06 (s, 6H) 0.71 (s, 9H) 0.99-1.14 (m, 2H) 1.25-1.30 (m, 3H) 1.30-1.42 (m, 2H) 1.78 (dd, J=28.30, 11.90 Hz, 5H) 2.20 (d, J=7.03 Hz, 2H) 2.28-2.39 (m, 1H) 3.64 (s, 3H) 3.83-3.97 (m, 2H) 4.04-4.14 (m, 1H) 7.00 (d, J=8.20 Hz, 2H) 7.19 (d, J=8.59 Hz, 2H) 8.53 (s, 1H).

### Preparation of Methyl 2-((1S,4s)-4-(4-((R)-4-chloro-8-methyl-5-oxo-7,8-dihydropyrimido-[5,4-f][1,4]oxazepin-6(5H)-yl)phenyl)cyclohexyl)acetate (I-1c-2)

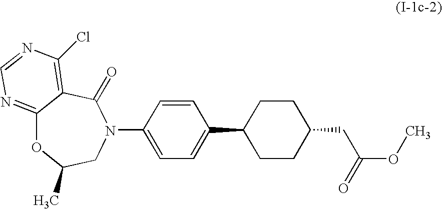

4M HCl in dioxane (25 mL) was added to a solution of methyl [trans-4-(4-{[(2R)-2-{[tert-butyl(dimethyl)silyl]oxy}propyl][(4,6-dichloropyrimidin-5-yl)carbonyl]amino}phenyl)cyclohexyl]acetate (I-1b-2: 3.95 g, 6.72 mmol), in methanol (50 mL). The mixture was stirred at 23° C. for 30 minutes. The reaction mixture was concentrated to remove the solvent. The residue was dissolved in acetonitrile (200 mL), then K₂CO₃ (1.86 g, 13.5 mmol) and 5 Angstrom molecular sieves (1.0 g) were added to it. The reaction mixture was stirred at 80° C. for 30 hours. EtOAc (250 mL) and water (250 mL) were added to reaction mixture. The organic layer was separated and dried over MgSO₄ and concentrated. The crude material was purified by a 120 g silica gel column eluted with 30-50% EtOAc in heptane to give a colorless oil 1.85 g (61%) as the title compound (I-1c-2).

m/z=444.1 (M+1). 1H NMR (400 MHz, chloroform-d) δ 1.09-1.23 (m, 2H) 1.43 (d, J=6.64 Hz, 3H) 1.44-1.57 (m, 2H) 1.80-1.96 (m, 5H) 2.26 (d, J=7.05 Hz, 2H) 2.44-2.55 (m, 1H) 3.68 (s, 3H) 3.80-3.95 (m, 2H) 5.00-5.12 (m, 1H) 7.29 (s, 4H) 8.76 (s, 1H).

### Preparation of Intermediate (trans-4-{4-[(8R)-4-amino-8-methyl-5-oxo-7,8-dihydropyrimido[5,4-f][1,4]oxazepin-6(5H)-yl]phenyl}cyclohexyl)acetic acid (I-1d-2)

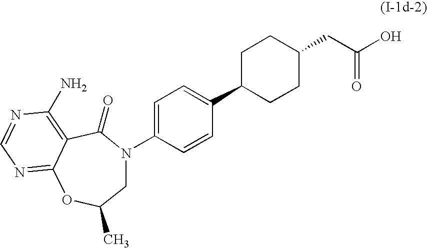

A mixture of methyl (trans-4-{4-[(8R)-4-chloro-8-methyl-5-oxo-7,8-dihydropyrimido[5,4-f][1,4]oxazepin-6(5H)-yl]phenyl}cyclohexyl)acetate (I-1c-2: 1.50 g, 3.38 mmol) in 0.5M ammonia in dioxane (20 mL) was stirred at 50° C., in a tightly capped flask, for 6 hours. The reaction mixture was concentrated to give a white solid, which was carried on to the next step without further purification. LiOH (247 mg, 9.89 mmol) was added to a solution of the white solid in THF/MeOH/water (30 mL, 3:2:1) and then the resulting solution was stirred at 23° C. for 18 hours. 1M HCl solution was added to reaction solution to adjust pH to about 3.2° C. i-propanol in DCM (130 mL) was added to extract reaction mixture. The organic layer was separated and dried over MgSO₄ and concentrated to give a solid. Purification was done by chromatography (80 g, silica gel column) with methanol/DCM from 2-6% to give a white solid 1210 mg (89%) as the title compound (I-1d-2).

m/z=411.1 (M+1). 1H NMR (400 MHz, METHANOL-d₄) δ ppm 1.11-1.25 (m, 2H) 1.36 (d, J=6.64 Hz, 3H) 1.53 (q, J=12.88 Hz, 2H) 1.75-1.96 (m, 5H) 2.21 (d, J=7.03 Hz, 2H) 2.46-2.58 (m, 1H) 3.80-3.96 (m, 2H) 4.92-5.03 (m, 1H) 7.25 (d, 2H) 7.31 (d, 2H) 8.17 (s, 1H).

### Preparation of Intermediate tert-butyl 4-[4-(4-amino-5-oxo-7,8-dihydropyrimido[5,4-f][1,4]oxazepin-6(5H)-yl)phenyl]piperidine-1-carboxylate (I-3a)

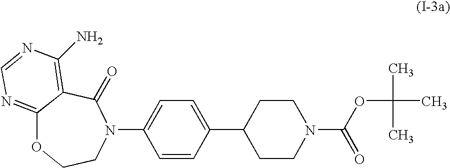

Intermediate I-3a was prepared according to procedures utilized to prepare (I-1e-1) utilizing tert-butyl 4-(4-trifluoromethylsulfonyloxy)phenyl)piperidine-1-carboxylate (prepared according to PCT Application No. WO2008075070 (Intermediate LL using 4-iodophenyl-trifluoromethylsulfonate as the starting material)) and 2-{[tent-butyl (dimethyl)silyl]oxy}ethanamine as the starting materials.

### Preparation of intermediate 4-[4-(4-Amino-5-oxo-7,8-dihydropyrimido[5,4-f][1,4]oxazepin-6(5H)-yl)phenyl]piperidine (I-3b)

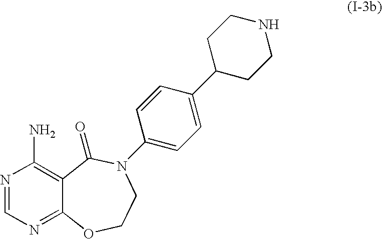

A solution of tent-butyl 4-[4-(4-amino-5-oxo-7,8-dihydropyrimido[5,4-f][1,4]oxazepin-6(5H)-yl)phenyl]piperidine-1-carboxylate (I-3a: 1.04 g, 2.37 mmol) and trifluoroacetic acid (7.4 mL) in dichloromethane (7.4 mL) was stirred at room temperature for 2 hours. The reaction solution was concentrated in vacuo, the residue diluted into 10% isopropyl alcohol:dichloromethane, washed with saturated aqueous sodium bicarbonate. The organic phase was concentrated in vacuo to afford the title compound (I-3b) as an off-white solid, 0.67 g.

1H NMR (400 MHz, CHLOROFORM-d) δ ppm 8.28 (s, 1H) 8.17 (br. s., 1H) 7.29 (d, 2H) 7.20 (d, 2H) 5.60 (br. s., 1H) 4.69-4.65 (m, 2H) 4.00-3.96 (m, 2H) 3.20-3.13 (m, 2H), 2.78-2.60 (m, 3H), 1.83-1.78 (m, 2H), 1.68-1.55 (m, 2H)

### Preparation of Intermediate 4-tert-butylphenyl trifluoromethanesulfonate (1C-1)

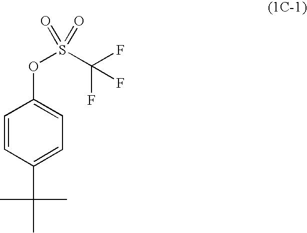

To a stirred solution of 4-tert-butylphenol (2.88 g, 19.2 mmol) and triethylamine (4.01 ml, 28.8 mmol) in dichloromethane (101 mL) was added a solution triflic anhydride (6.8 g, 24 mmol) drop wise. The mixture was continued to stir at 0° C. for 2 hrs. The reaction mixture was washed with water and brine and dried over sodium sulfate, filtered and concentrated to give a dark brown oil. Product was purified on silica gel eluting with heptane to give 4-tert-butylphenyl trifluoromethanesulfonate (1C-1) (3.64 g 67.3%) as a clear oil.

1H NMR (400 MHz, CHLOROFORM-d) d ppm 1.31 (s, 9H) 7.17 (d, J=8.72 Hz, 2H) 7.43 (d, J=8.72 Hz, 2H)

### Preparation of Intermediate methyl 2-(4-bromophenyl)-2-methylpropanoate (1D-1)

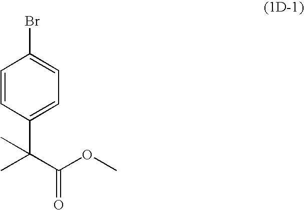

A solution of 4-bromophenylacetic acid (10 g, 47 mmol) in methanol (194 ml, 46.5M) and sulfuric acid (2.48 ml, 46.5 mmol) was heated to reflux for 16 hours. Reaction was concentrated, diluted with ethyl acetate and washed with saturated sodium bicarbonate and brine. Organic was dried over sodium sulfate, filtered and concentrated to give methyl 2-(4-bromophenyl)acetate (10.63 g, 100%) as a colorless oil.

1H NMR (400 MHz, CHLOROFORM-d) d ppm 3.56 (s, 2H) 3.68 (s, 3H) 7.14 (d, J=8.59 Hz, 2H) 7.43 (d, J=8.59 Hz, 2H)

A solution of methyl 2-(4-bromophenyl)acetate (6 g, 30 mmol) in tetrahydrofuran (67.2 ml, 0.39M) was added 1M potassium t-butoxide in tetrahydrofuran (57.6 ml, 57.6 mmol). Reaction mixture was cooled to 0° C. and methyl iodide (3.59 ml, 57.6 mmol) was added drop wise. After addition was complete, reaction was slowly warmed up to room temperature and stirred for 16 hours. Reaction mixture was then carefully quenched with 1M hydrochloric acid and concentrated. Reaction was diluted with water and extracted with ethyl acetate. Pooled organics were washed with water and brine and then dried over sodium sulfate, filtered and concentrated to give a crude dark oil. Crude product purified on silica gel eluting with 0%-5% ethyl acetate in heptane to give methyl 2-(4-bromophenyl)-2-methylpropanoate (1D-1) (6.44 g, 92%) as a yellow oil

1H NMR (400 MHz, CHLOROFORM-d) d ppm 1.54 (s, 6H) 3.63 (s, 3H) 7.19 (d, J=8.78 Hz, 2H) 7.43 (d, J=8.98 Hz, 2H)

MS (LC-MS) 371.2 (M+1)

### Preparation of Intermediate 1-(4-bromophenyl)cyclobutanol (1O-1)

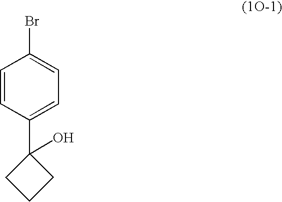

1-Bromo-4-iodobenzene (1.6293 g, 5.75 mmol) dissolved in tetrahydrofuran (10 mL). Reaction cooled to −78° C. and n-butyl lithium (2.5M solution in hexane, 2.42 mL, 6.05 mmol) added and continued to stir at −78° C. for 20 minutes. Cyclopentanone (0.448 mL, 6.05 mmol) added to cold solution and once addition was complete, reaction was warmed to room temperature and stirred for 16 hours. Reaction was diluted with aqueous saturated ammonium chloride and extracted with a 1:1 solution of ethyl acetate in tetrahydrofuran. Pooled organics dried over sodium sulfate, filtered and concentrated to give a thick oil. Oil purified on silica gel eluting with a gradient from 0% to 30% ethyl acetate in heptane to give 1-(4-bromophenyl)cyclobutanol (0.8033 g, 65%) as a clear oil. US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Compounds of the invention and combination therapies may be administered in conjunction with exercise and a sensible diet.

Embodiments of the invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England).

### General Experimental Procedures

NMR spectra were recorded on a Varian Unity™ 400 (available from Varian Inc., Palo Alto, Calif.) at room temperature at 400 MHz for proton. Chemical shifts are expressed in parts per million (δ) relative to residual solvent as an internal reference. The peak shapes are denoted as follows: s, singlet; d, doublet; dd, doublet of doublet; t, triplet; q, quartet; m, multiplet; bs, broad singlet; 2s, two singlets. Atmospheric pressure chemical ionization mass spectra (APCI) were obtained on a Fisons™ Platform II Spectrometer (carrier gas: acetonitrile: available from Micromass Ltd, Manchester, UK). Chemical ionization mass spectra (CI) were obtained on a Hewlett-Packard™ 5989 instrument (ammonia ionization, PBMS: available from Hewlett-Packard Company, Palo Alto, Calif.). Electrospray ionization mass spectra (ES) were obtained on a Waters™ ZMD instrument (carrier gas: acetonitrile: available from Waters Corp., Milford, Mass.). High resolution mass spectra (HRMS) were obtained on an Agilent™ Model 6210 using time of flight method. Where the intensity of chlorine or bromine-containing ions are described, the expected intensity ratio was observed (approximately 3:1 for ³⁵Cl/³⁷Cl-containing ions and 1:1 for ⁷⁹Br/⁸¹Br-containing ions) and the intensity of only the lower mass ion is given. In some cases only representative ¹H NMR peaks are given. Optical rotations were determined on a PerkinElmer™ 241 polarimeter (available from PerkinElmer Inc.,

### Preparation of Intermediate 3-(4-bromophenyl)cyclobutanol (1Q-1)

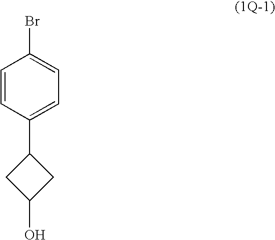

To a stirred mixture of dimethyl acetamide (6.6 mL, 71 mmol) and dichloroethane (50 mL) was added triflic anhydride (11.9 mL, 70.9 mmol) drop wise over 10 minutes at −12° C. for 25 minutes. 1-Bromo-4-vinylbenzene (8.4 mL, 64.46 mmol) was added followed by slow addition of 2,4,6-collidine. Reaction mixture was then heated to 150° C. for 4 hours. Water (60 ml) was added, and the mixture was stirred at 80° C. for 20 hours. Reaction mixture was cooled to room temperature and water (40 mL) and ethyl acetate (200 mL) added. The organic phase was separated, washed with brine, dried over magnesium sulfate, filtered and concentrated. The obtained dark brown residue was extracted with toluene (2×250 ml) and concentrated. Crude residue was purified on silica gel, eluting with a gradient from 0% to 20% ethyl acetate in heptane to give 3-(4-bromophenyl)cyclobutanone.

1H NMR (CHLOROFORM-d) Shift: 7.46 (d, J=8.4 Hz, 2H), 7.16 (d, J=8.4 Hz, 2H), 3.57-3.67 (m, 1H), 3.43-3.54 (m, 2H), 3.13-3.25 (m, 2H)

Sodium borohydride (1.23 g mg, 32.5 mmol) was added to a solution of 3-(4-bromophenyl)cyclobutanone (6.65 g, 29.5 mmol) in tetrahydrofuran (50 mL) at 0° C. The reaction was stirred at room temperature for 1 hour. Saturated sodium bicarbonate added and stirred at room temperature for 1 hour. Extracted with a 1:1 solution of ethyl acetate in heptane. The extract was washed with brine, dried over magnesium sulfate and concentrated to obtain 3-(4-bromophenyl)cyclobutanol (1Q-1) (6.4 g, 95%) as a mixture of cis and trans isomers, which will be used for the next step without purification.

### Preparation of Intermediate 2,2,2-trifluoro-1-(4-iodophenyl)ethanol (1R-1)

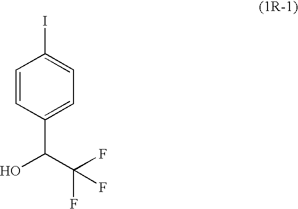

2,2,2-Trifluoro-1-(4-iodophenyl)ethanone (1.8 g, 6 mmol) was dissolved in methanol (60 mL) and cooled to 0° C. Sodium borohydride (0.227 g, 6 mmol) added and reaction stirred at 0° C. for 3 hours. Saturated aqueous ammonium chloride was added and the reaction mixture was extracted with ethyl acetate. Organic was washed with water (2 mL), dried over sodium sulfate, filtered and concentrated. Crude purified on silica gel eluting with a gradient from 3% to 20% ethyl acetate in heptane to give 2,2,2-trifluoro-1-(4-iodophenyl)ethanol (1.5 g, 82%). GCMS was 302 at 2.11 min.

1H NMR (400 MHz, CHLOROFORM-d) d ppm 2.65 (d, J=4.49 Hz, 1H) 4.91-5.02 (m, 1H) 7.20 (d, J=8.39 Hz, 2H) 7.74 (d, J=8.39 Hz, 2H)

To a solution of tert-butyldimethylsilyl chloride (686 mg, 4.55 mmol), 4-dimethylaminopyridine (50.6 mg, 0.414 mmol) and triethylamine (0.865 mL, 6.21 mmol) in dichloromethane (20 mL), a solution of 2,2,2-trifluoro-1-(4-iodophenyl)ethanol in 5 ml of dichloromethane was added drop wise at room temperature. Stirred for 24 hours. Reaction was concentrated and water (50 mL) added. Solution extracted with ethyl acetate (100 mL) and organic layer washed with brine, dried over magnesium sulfate, filtered and concentrated. Crude purified on silica gel, eluting with 0% to 10% ethyl acetate in heptane to give tert-butyldimethyl(2,2,2-trifluoro-1-(4-iodophenyl)ethoxy)silane (450 mg, 26%) as a colorless oil.

1H NMR (400 MHz, CHLOROFORM-d) d ppm −0.03 (s, 3H) 0.10 (s, 3H) 0.88 (s, 9H) 4.84 (q, J=6.44 Hz, 1H) 7.15-7.18 (m, 1H) 7.18-7.20 (m, 1H) 7.68-7.71 (m, 1H) 7.71-7.74 (m, 1H).

### Preparation of Intermediate 1-bromo-4-(3,3-difluorocyclobutyl)benzene (1T-1)

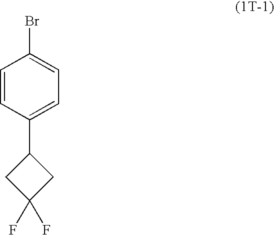

3-(4-Bromophenyl)cyclobutanone (600 mg, 2.67 mmol) was dissolved in dichloromethane (10 mL) and toluene (10 mL). Boron trifluoride diethyl etherate (0.676 mL, 5.33 mmol) was added and reaction cooled to 0° C. Deoxo-Fluor® (0.983 mL, 5.33 mmol) was added drop wise and once addition was complete, the reaction was warmed to room temperature for 48 hours. 1M aqueous sodium hydroxide (10 ml) was added and vigorously stirred for 30 minutes. Reaction was extracted with dichloromethane (50 mL), dried over sodium sulfate, filtered and concentrated. Crude purified on silica gel, eluting with a gradient from 0% to 8% ethyl acetate in heptane to give 1-bromo-4-(3,3-difluorocyclobutyl)benzene (360 mg, 54%) as a colorless oil.

GCMS was 248 at 1.94 min.

1H NMR (400 MHz, CHLOROFORM-d) d ppm 2.53-2.72 (m, 2H) 2.92-3.07 (m, 2H) 3.26-3.40 (m, 1H) 7.06-7.13 (m, 2H) 7.41-7.49 (m, 2H)

### Preparation of Intermediate tert-butyl(1,1,1,3,3,3-hexafluoro-2-(4-iodophenyl)propan-2-yloxy)dimethylsilane (1V-1)

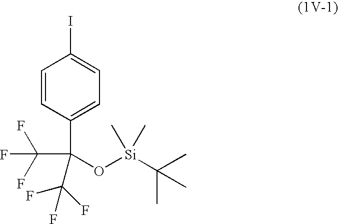

4-Iodobenzoic acid methyl ester (5 g, 19.08 mmol) dissolved in tetrahydrofuran (80 mL) and cooled to 0° C. (Trifluoromethyl)trimethylsilane (5.43 g, 38.2 mmol) and cesium fluoride (145 mg, 0.954 mmol) added. Once addition was complete, reaction was warmed up to room temperature and stirred for 3 hours. Additional (trifluoromethyl)trimethylsilane (2.715 g, 19.08 mmol) was added and reaction stirred at room temperature for 4 hours. 4 M aqueous solution of hydrochloric acid (20 mL) added and stirred for 5 hours. The reaction mixture was diluted with ethyl acetate (500 ml), washed with water (2×250 ml), dried over sodium sulfate, filtered and concentrated. Crude purified on silica gel eluting with a gradient from 0% to 10% ethyl acetate in heptane to afford 2,2,2-trifluoro-1-(4-iodophenyl)ethanone (1.8 g, 31%) GCMS=300 at 1.47 min and 1,1,1,3,3,3-hexafluoro-2-(4-iodophenyl)propan-2-ol (1.6 g, 22%); GCMS=370 at 1.60 min.

To a solution of tert-butyldimethylsilyl chloride (686 mg, 4.55 mmol), 4-dimethylaminopyridine (50.5 mg, 0.413 mmol) and triethylamine (0.864 mL, 6.2 mmol) in dichloromethane (20 mL), a solution of 1,1,1,3,3,3-hexafluoro-2-(4-iodophenyl)propan-2 of in 5 ml of dichloromethane was added drop wise at room temperature. Stirred for 24 hours. Reaction was concentrated and water (50 mL) added. Solution extracted with ethyl acetate (100 mL) and organic layer washed with brine, dried over magnesium sulfate, filtered and concentrated. Crude purified on silica gel, eluting with 0% to 10% ethyl acetate in heptane to give tert-butyl(1,1,1,3,3,3-hexafluoro-2-(4-iodophenyl)propan-2-yloxy)dimethylsilane (2 g, 99%) as a colorless oil.

1H NMR (400 MHz, CHLOROFORM-d) d ppm 0.15 (s, 6H) 0.98 (s, 9H) 7.41 (d, J=8.78 Hz, 2H) 7.76 (d, J=8.98 Hz, 2H)

### Preparation of Intermediate (1-(4-bromophenyl)ethoxy)(tert-butyl)dimethylsilane (1W-1)

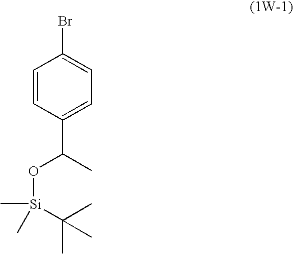

4-Bromo-alpha-methylbenzyl alcohol (3 g, 10 mmol), imidazole (1.97 mL, 29.8 mmol) and tert-butyl-chlorodimethylsilyl chloride (3 g, 19.3 mmol) combined in dimethylformamide (37 mL) and stirred at room temperature for 16 hours. Water and diethyl ether added and stirred vigorously. Organic layer was separated and washed with brine, dried over sodium sulfate, filtered and concentrated to give a crude oil that was purified on silica gel eluting with 1% ethyl acetate in heptane to give (1W-1) (4.4 g, 90%) as a colorless oil.

### Preparation of Intermediate 1-iodo-4-isobutylbenzene (1Y-1)

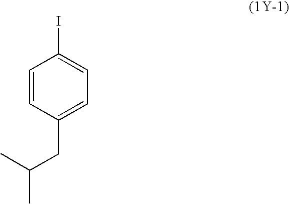

1-Isobutylbenzene (5 g, 37 mmol) was added to a mixture of iodine (9.46 g, 37.3 mmol) and silver(I) nitrite (5.85 g, 37.3 mmol) in dichloromethane (200 mL) at room temperature. Reaction was stirred for 96 hours. Yellow solid was filtered off and the filtrate was Washed with 10% aqueous sodium sulfite (500 mL), saturated aqueous sodium bicarbonate and brine and dried over magnesium sulfate, filtered and concentrated. Crude was purified on silica gel, eluting with a gradient from 0% to 5% ethyl acetate in heptane to give 1-iodo-4-isobutylbenzene (7 g, 70%) as a pink oil.

### Preparation of Intermediate 4-(1,1,1-trifluoro-2-methylpropan-2-yl)phenol (1Z-1)

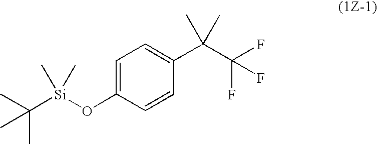

Piperidine (99.8 mL, 1.01 mol, 1.25 eq) and triethylamine (120.8 mL, 0.81 mol, 1.0 eq) in ether (394 mL) were cooled to 0° C. and trifluoroacetic anhydride (120.8 mL, 0.81 mol, 1.0 eq) in ether (263 mL) was added drop wise over 30 minutes. The reaction was warmed to room temperature and stirred for 16 hours. The reaction was diluted with ether (625 mL) and washed with 0.2 N aqueous hydrochloric acid until neutral. The organic portion was washed with brine, dried over sodium sulfate and concentrated. The resulting yellow oil was purified on silica gel eluting with 10% ethyl acetate in hexane to give 2,2,2-trifluoro-1-(piperidin-1-yl)ethanone (140.35 g, 77%).

1H NMR (CDCl3, 400 MHz): 3.61 (2H, m), 3.52 (2H, m), 1.67 (6H, m).

Magnesium turnings (7.73 g, 318 mmol, 1.25 eq) and tetrahydrofuran (63 mL) were placed in a 3 neck flask. 4-Bromoanisole (59.40 g, 318 mmol, 1.25 eq) in tetrahydrofuran (63 mL) was added drop wise and the flask heated until a vigorous reaction occurred. Once the magnesium had dissolved the reaction was cooled to 0° C. and 2,2,2-trifluoro-1-(piperidin-1-yl)ethanone (46.00 g, 258 mmol) in tetrahydrofuran (250 mL) was added drop wise. The reaction was stirred at room temperature for 2 hours and was subsequently quenched with saturated aqueous ammonium chloride and the resulting precipitate filtered off. The filtrate was dried over sodium sulfate concentrated to give an orange oil which was purified by distillation (120 C, 32 mbar) to give 2,2,2-trifluoro-1-(4-methoxyphenyl)ethanone (80 g, 52%).

2,2,2-Trifluoro-1-(4-methoxyphenyl)ethanone (80.00 g, 392 mmol) in diethyl ether (800 mL) was cooled to 0° C. Methyl magnesium bromide (3.0M in diethyl ether, 130.4 mL, 392 mmol, 1.0 eq) was added drop wise and the reaction allowed to warm to room temperature overnight. The reaction was quenched with 1N hydrochloric acid (800 mL), the layers separated and the organic portion washed with water (800 mL) dried over sodium sulfate and concentrated to give 1,1,1-trifluoro-2-(4-methoxyphenyl)propan-2-ol (85 g, 98%) as a yellow oil.

1H NMR (CDCl3, 400 MHz): 7.50 (2H, d), 6.91 (2H, d), 3.81 (3H, s), 2.33 (1H, bs), 1.75 (3H, s). 00 MHz): 8.05 (2H, d), 7.00 (2H, d), 3.90 (3H, s).

1,1,1-Trifluoro-2-(4-methoxyphenyl)propan-2-ol (85.00 g, 391 mmol) in dichloromethane (860 mL) was cooled to 0° C. and titanium tetrachloride (40.52 mL, 1.0 eq) was added slowly to the reaction. The reaction was stirred at 0° C. for 1.5 hours and was then added slowly to ice water and the layers were separated and the aqueous portion extracted with dichloromethane (3×500 mL). The combined organics were washed with saturated sodium hydrogen carbonate and brine, dried over sodium sulfate and concentrated. The crude oil was purified on silica gel eluting with hexane to give 1-(2-chloro-1,1,1-trifluoropropan-2-yl)-4-methoxybenzene (60.9 g, 65%).

1H NMR (CDCl3, 400 MHz): 7.58 (2H, d), 6.89 (2H, d), 3.78 (3H, s), 2.11 (3H, s).

Trimethyl aluminium (2.0 M in heptane, 504 mL, 1.04 mol, 4 eq) was added to 1-(2-chloro-1,1,1-trifluoropropan-2-yl)-4-methoxybenzene (60.00 g, 251 mmol) in hexane (840 mL). The reaction was heated at reflux for 2 hours. The reaction was cooled and quenched slowly with 2N hydrochloric acid. The layers were separated and the aqueous portion extracted with hexane. The organic portion was dried over sodium sulfate and concentrated to give 1-methoxy-4-(1,1,1-trifluoro-2-methylpropan-2-yl)benzene (32.09 g, 58%).

1H NMR (CDCl3, 400 MHz): 7.42 (2H, d), 6.90 (2H, d), 3.79 (3H, s), 1.55 (6H, s).

1-Methoxy-4-(1,1,1-trifluoro-2-methylpropan-2-yl)benzene (32.00 g, 147 mmol) in dichloromethane (500 mL) was cooled to 0° C. Boron tribromide (14.14 mL, 147 mmol, 1.0 eq) was added drop wise. The reaction was allowed to warm to room temperature and was stirred for 4 hours. The reaction was then cooled to 0° C. and quenched by the slow addition of water. The layers were separated and the aqueous portion extracted with dichloromethane. The combined organic extracts were washed with brine and dried over sodium sulfate and concentrated. Crude was purified on silica gel eluting with 5% ethyl acetate in hexane to give 4-(1,1,1-trifluoro-2-methylpropan-2-yl)phenol (1Z-1) (29.01 g, 97%).

1H NMR (CDCl3, 400 MHz): 7.34 (2H, d), 6.82 (2H, d), 1.53 (6H, s).

### Preparation of Intermediate 1-bromo-4-(1-methoxy-2-methylpropan-2-yl)benzene (1AA-1)

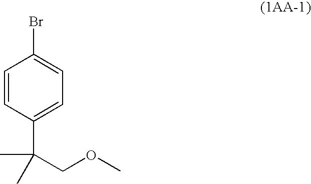

Methyl 2-(4-bromophenyl)-2-methylpropanoate (23.5 g, 86.6 mmol) in tetrahydrofuran (175 mL) was cooled to −78° C. Lithium aluminum hydride (100 mL of 1.0 M solution) added slowly over 45 minutes and stirred for 3 hours at cold temperature. Reaction solution was slowly diluted with ethyl acetate and stirred for 10 minutes. 1M hydrochloric acid was slowly added drop wise to reaction mixture. Diethyl ether (200 mL) added and layers separated. Organic washed with 1 M hydrochloric acid, brine, dried over sodium sulfate, filtered and concentrated to give 2-(4-bromophenyl)-2-methylpropan-1-ol (20 m, 100%) as a white solid.

A suspension of sodium hydride (60% in mineral oil, 637 mg, 15.9 mmol) in tetrahydrofuran (133 mL) was cooled to 0° C. To this mixture was added 2-(4-bromophenyl)-2-methylpropan-1-ol (3.04 g, 13.3 mmol) in tetrahydrofuran (10 mL) drop wise. Once addition was complete, reaction was slowly warmed to room temperature and stirred for 2 hours. Reaction was then cooled to 0° C. and methyl iodide (1.26 mL, 19.9 mmol) was added drop wise. The suspension was stirred at 0° C. for 3 hours and then warmed up to room temperature for 18 hours. Saturated aqueous ammonium chloride added to reaction mixture and layers separated. Organic washed with brine, dried over sodium sulfate, filtered and concentrated. Crude purified on silica gel eluting with 35% ethyl acetate in heptane to give (1AA-1) (2.62, 81%) as a pale oil.

1H NMR (400 MHz, CHLOROFORM-d) d ppm 1.10 (s, 6H) 2.70 (s, 2H) 3.24 (s, 3H) 7.05 (d, J=8.59 Hz, 2H) 7.37 (d, J=8.39 Hz, 2H)

### Preparation of Intermediate (1-(4-bromophenyl)-2,2-dimethylpropoxy)(tert-butyl)dimethylsilane (1-AC-1)

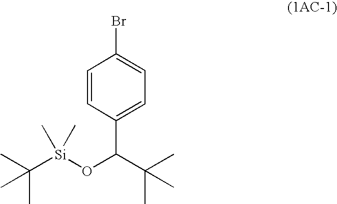

t-butyl magnesium chloride in tetrahydrofuran (9 mL, 8.11 mmol) was added to a solution of 4-bromo benzaldehyde (1 g, 5.4 mmol) in tetrahydrofuran (20 mL) at 0° C. Once addition was complete, reaction warmed up to room temperature and stirred for 18 hours. Saturated aqueous ammonium chloride (10 mL) added and extracted with ethyl acetate. The organic layer was separated and dried over magnesium sulfate and concentrated. Crude purified on silica gel eluting with a gradient from 3% to 10% ethyl acetate in heptane to give 1-(4-bromophenyl)-2,2-dimethylpropan-1-ol (630 mg, 47%) as a colorless oil.

1H NMR (400 MHz, CHLOROFORM-d) d ppm 0.89 (s, 9H) 1.85 (s, 1H) 4.34 (s, 1H) 7.16 (d, J=8.20 Hz, 2H) 7.42 (d, J=8.39 Hz, 2H)

To a solution of 1-(4-bromophenyl)-2,2-dimethylpropan-1-ol (630 mg, 2.59 mmol) in dimethylformamide at room temperature under nitrogen was added tert-butyldimethylsilyl chloride (818 mg, 5.43 mmol) and stirred for 65 hours. Reaction mixture was concentrated, diluted with water (50 mL) and extracted with 1:1 ethyl acetate and heptane (100 mL). The organic phase was separated, washed with brine, dried over magnesium sulfate, and concentrated to give (1-(4-bromophenyl)-2,2-dimethylpropoxy)(tert-butyl)dimethylsilane (1AC-1) (900 mg, 97%) as a colorless oil.

### Preparation of Intermediate methyl 1-(4-bromophenyl)cyclohexanecarboxylate (1AE-1)

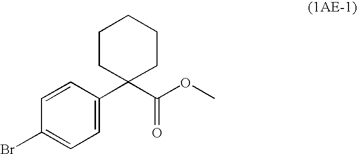

Sodium hydride (12 g, 52 mmol) was suspended in tetrahydrofuran (200 mL) under argon and warmed to 35° C. Methyl 2-(4-bromophenyl)acetate (26 mmol) in tetrahydrofuran added drop wise to reaction over 1 hour. The reaction mixture was then kept at this temperature for 1 hour until all gas evolution has ceased. The 1,5-diiodopentane (17 g, 52 mmol) was then added drop wise as a solution in tetrahydrofuran (100 mL) and the reaction mixture stirred at 35° C. for a further hour and at ambient temperature overnight. After this time, the reaction mixture was cooled to 0° C. and quenched by the addition of dry silica, filtered and the solvent removed under vacuum. The crude product was then purified by flash chromatography eluting with 33% ethyl acetate in heptane to give methyl 1-(4-bromophenyl)cyclohexanecarboxylate (1AC-1) (15.3 g, 99% yield) as a yellow oil.

1H NMR (400 MHz, CDCl3): 7.45-7.38 (m, 2H), 7.27-7.24 (m, 2H), 3.63 (s, 3H), 2.43 (d, J=13.3 Hz, 2H), 1.71-0.80 (m, 8H) ppm.

### Preparation of Intermediate methyl 1-(4-bromophenyl)cyclopentanecarboxylate (1AF-1)

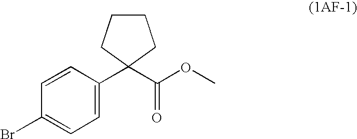

Methyl 2-(4-bromophenyl)acetate (73.0 g, 0.32 mol) was dissolved in tetrahydrofuran (750 mL) and 1,4-diiodobutane (25.5 g, 0.64 mol) was added. The mixture was stirred under a flow of argon and sodium hydride (60% on oil, 100.0 g, 0.32 mol) was added slowly in portions. After the addition was complete, the mixture was stirred at room temperature for 16 hours. The mixture was poured onto ice-cold water (500 mL) and ethyl acetate was added (500 mL). The mixture was separated and the aqueous layer washed with ethyl acetate (500 mL). The organic layers were combined and washed with brine (1 L), dried over magnesium sulfate and concentrated to give methyl 1-(4-bromophenyl)cyclopentanecarboxylate (42.0 g, 47%) as a yellow solid.

1H NMR (CDCl, 400 MHz): 7.41 (d, 2H), 7.22 (d, 2H), 3.59 (s, 3H), 2.55-2.66 (m, 2H), 1.81-1.90 (m, 2H), 1.68-1.75 (m, 4H).

### Preparation of Intermediate 1-bromo-4-(2-methoxy-2-methylpropyl)benzene (1AG-1)

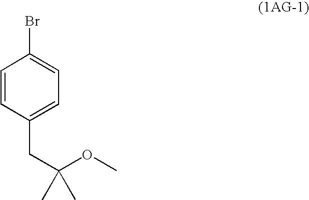

2-(4-Bromophenyl)acetic acid (75 g, 340 mmol) suspended in ethanol (341 mL). Concentrated sulfuric acid (0.682 mL, 12.79 mmol) was added and reaction heated to reflux for 24 hours. Reaction concentrated and residue diluted with diethyl ether and saturation sodium bicarbonate. Layers carefully separated and organic was washed with brine, dried over sodium sulfate, filtered and concentrated to give ethyl 2-(4-bromophenyl)acetate (80.2 g, 97%) as off-white solid.

Methyl magnesium bromide (3M in tetrahydrofuran; 10.6 mL, 31.8 mmol) in tetrahydrofuran (10 mL) was cooled to 0° C. Ethyl 2-(4-bromophenyl)acetate (2.58 g, 10.6 mmol) in tetrahydrofuran (30 mL) was added drop wise to cold reaction over 15 minutes. Stirred at 0° C. for 3 hours. Reaction was carefully quenched with aqueous saturated ammonium chloride and then acidified with 1M hydrochloric acid. The reaction mixture was diluted with diethyl ether and layers separated. Organic washed with brine, dried over sodium sulfate, filtered and concentrated to give (1AG-1) (2.34 g, 96%) as a clear oil.

1H NMR (500 MHz, CHLOROFORM-d) d ppm 1.23 (s, 6H) 1.31 (br. s., 1H) 2.74 (s, 2H) 7.11 (d, J=8.05 Hz, 2H) 7.45 (d, J=8.29 Hz, 2H)

### Preparation of Intermediate methyl 1-(4-bromophenyl)cyclobutanecarboxylate (1AH-1)

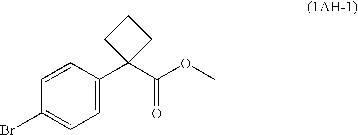

Sodium hydride (3.5 g, 88 mmol) was stirred as a suspension in dimethylformamide (250 ml) under argon. This was warmed to 35° C. and methyl 2-(4-bromophenyl)acetate (10 g, 44 mmol) in dimethylformamide (100 mL) was added drop wise over 1 hour and then stirred at 30° C. for 1 hour. To this the 1,3-dibromopropane (4.4 ml, 44 mmol) in dimethylformamide (50 ml) was added drop wise over 1 hour, and this was left to stir at room temperature overnight. The reaction was incomplete. Sodium hydride (3.5 g, 88 mmol) was prepared in dimethylformamide (100 ml) at 35° C. and was added to this drop wise to the reaction mixture over 1 hour. This was again left to stir at room temperature overnight. Saturated aqueous ammonium chloride solution (200 ml) was carefully added, followed by water (500 ml). The product was extracted with ethyl acetate (2×500 ml), washed with water (3×500 ml), and brine (2×500 ml). The organic solution was then dried over magnesium sulfate, filtered, and evaporated. The crude product was purified by flash chromatography (12.5% ethyl acetate in heptane) to methyl 1-(4-bromophenyl)cyclobutanecarboxylate (900 mg, 3.3 mmol, 7.5%).

1H NMR (400 MHz CDCl3) 7.45 (d, 2H), 7.15 (d, 2H), 3.65 (s, 3H), 2.80 (m, 2H), 2.45 (m, 2H), 2.05 (m 1H), 1.85 (m, 1H)

### Preparation of Intermediate 2-(4-bromophenyl)-2-ethylbutan-1-ol (1AI-1)

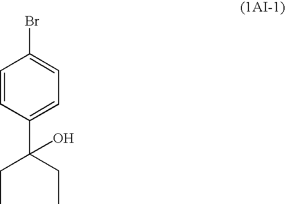

Dibromobenzene (3 g, 12.72 mmol) tetrahydrofuran (30 mL) was cooled to −78° C. and n-butyllithium in hexane (896 mg, 14 mmol) was added drop wise. Pentan-3-one (1.31 g, 15.3 mmol) added drop wise and continued to stir at cold temperature for 3 hours. Saturated aqueous ammonium chloride and water were added and reaction was extracted with 75% ethyl acetate in heptane. Organic washed with brine, dried over magnesium sulfate, filtered and concentrated to give (1AI-1) (2.44 g, 78%) as a colorless oil.

1H NMR (400 MHz, CHLOROFORM-d) d ppm 0.73 (t, 6H) 1.57 (s, 1H) 1.71-1.88 (m, 4H) 7.23 (d, 2H) 7.43 (d, 2H)

### Preparation of Intermediate 1-(4-bromophenyl)cyclohexanol (1AJ-1)

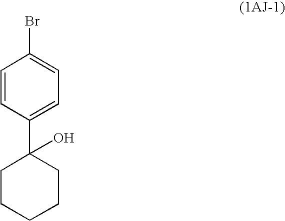

Dibromobenzene (3 g, 12.72 mmol) dissolved in tetrahydrofuran (35 mL) and cooled to −78° C. A 2.5M solution of n-butyllithium in hexane (5.6 mL, 14 mmol) added drop wise to cold reaction mixture and stirred at −78° C. for 1 hour. Cyclohexanone (1.45 mL, 14 mmol) was added drop wise at −78° C. Once addition was complete, reaction was warmed up to 0° C. for 1 hour. Saturated aqueous ammonium chloride and water added. Reaction mixture extracted with a 2:1 solution of ethyl acetate:heptane and organic layers washed with brine, dried over magnesium sulfate, filtered and concentrated to give 1-(4-bromophenyl)cyclohexanol (3.2 g, 98%) as a colorless oil.

1H NMR (400 MHz, CHLOROFORM-d) d ppm 1.21-1.34 (m, 1H) 1.56-1.89 (m, 10H) 7.36 (d, 2H) 7.44 (d, 2H)

### Preparation of Intermediate 2-(4-bromophenyl)propan-2-ol (1AK-1)

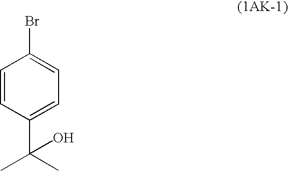

Dibromobenzene (2 g, 8.478 mmol) in tetrahydrofuran (25 mL) was cooled to −78° C. and n-butyllithium (2.5 M in hexane; 3.8 mL, 9.33 mmol) was added drop wise and stirred for 1 hour. Acetone (591 mg, 10.2 mmol) added drop wise and once addition was complete, the reaction was warmed up to 0° C. and stirred for 3 hours. Saturated ammonium chloride and water added and extracted with 75% ethyl acetate in heptane. Organic washed with brine, dried over magnesium sulfate, filtered and concentrated to give (1AK-1) (1.74 g, 95%) as a colorless oil.

1H NMR (500 MHz, CHLOROFORM-d) d ppm 1.58 (s, 6H) 1.79-1.82 (m, 1H) 7.38 (d, 2H) 7.47 (d, 2H)

### Example 1

### Preparation of 4-amino-6-{4-[trans-4-(2-hydroxy-2-methylpropyl)cyclohexyl]phenyl}-7,8-dihydropyrimido[5,4-f][1,4]oxazepin-5(6H)-one (1A)

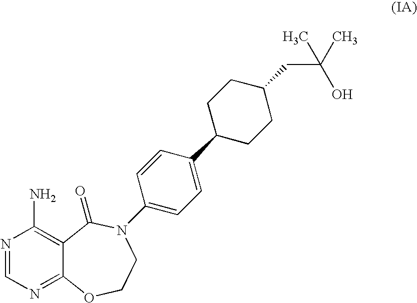

To an ice cooled solution of methyl {trans-4-[4-(4-amino-5-oxo-7,8-dihydropyrimido[5,4-f][1,4]oxazepin-6-(5H)-yl)phenyl]cyclohexyl}acetate (I-1e-1: 40 mg, 0.10 mmol) was added methyl magnesium bromide (1.4 M in toluene, 0.83 mL, 1.16 mmol), the cooling bath was allowed to expire and the reaction mixture was stirred for 24 hours. The reaction was partitioned between water and ethyl acetate, the organic phase dried over sodium sulfate and concentrated in vacuo. Chromatography on silica gel (4 g, 1-5% methanol:dichloromethane) afforded the title compound (IA) as a white solid, 10 mg.

1H NMR (400 MHz, CHLOROFORM-d) δ ppm 8.25 (s, 1H) 8.15 (br. s., 1H) 7.22-7.30 (m, 2H) 7.12-7.19 (m, 2H) 5.71 (br. s., 1H) 4.63-4.69 (m, 2H) 3.94-4.03 (m, 2H) 2.41-2.53 (m, 1H) 1.82-1.98 (m, 4H) 1.38-1.56 (m, 5H) 1.04-1.28 (m, 8H). m/z=411.4 (M+1).

### Example 1B

### Preparation of 4-amino-6-{4-[trans-4-(2-amino-2-methylpropyl)cyclohexyl]phenyl}-7,8-dihydropyrimido[5,4-f][1,4]oxazepin-5(6H)-one (1B)

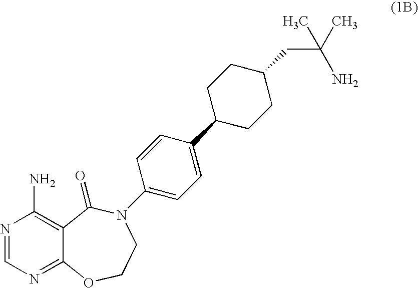

To a stirred solution of 4-amino-6-{4-[trans-4-(2-hydroxy-2-methylpropyl)cyclohexyl]phenyl}-7,8-dihydropyrimido[5,4-f][1,4]oxazepin-5(6H)-one (1A: 100 mg, 0.24 mmole) and trimethylsilylazide (42 mg, 0.37 mmole) was added boron trifluoride etherate (54 mg, 0.37 mmole) dropwise. After 30 hours the reaction mixture was partitioned between ethyl acetate and saturated aqueous sodium bicarbonate. The organic layer was separated, dried over sodium sulfate to afford a white solid (106 mg), which was taken onto the next step without further purification.

The material prepared in the previous step was dissolved in ethyl acetate (10 mL)/ethanol (10 mL), 10% palladium-on-carbon (25 mg) was added and the slurry was shaken under an atmosphere of hydrogen gas (50 p.s.i.) for 20 hours. The reaction mixture was filtered through a pad of Celite, washing with ethyl acetate and the combined filtrates were concentrated in vacuo. Chromatography on silica gel utilizing a gradient of 3-10% of 10% ammonium hydroxide in methanol:dichloromethane afforded the title compound (1B) as a white solid, 21 mg.

1H NMR (400 MHz, METHANOL-d4) δ ppm 1.14 (s, 6H) 1.16-1.30 (m, 3H) 1.36 (d, J=4.98 Hz, 2H) 1.40-1.62 (m, 3H) 1.88 (dd, J=23.06, 12.25 Hz, 3H) 2.43-2.57 (m, 1H) 3.94-4.04 (m, 2H) 4.61-4.72 (m, 2H) 7.17-7.26 (m, 2H) 7.26-7.33 (m, 2H) 8.14 (s, 1H). m/z=410.0 (M+1).

The compounds listed in Table 1 below were prepared using procedures analogous to those described above for the synthesis of Intermediate Methyl {trans-4-[4-(4-amino-5-oxo-7,8-dihydropyrimido[5,4-f][1,4]oxazepin-6-(5H)-yl)phenyl]cyclohexyl}acetate (I-1e-1) or, when R² is methyl, intermediate Methyl 2-((1S,4s)-4-(4-((R)-4-chloro-8-methyl-5-oxo-7,8-dihydropyrimido-[5,4-f][1,4]oxazepin-6(5H)-yl)phenyl)cyclohexyl)acetate (I-1c-2) using the appropriate starting materials which are available commercially, prepared using preparations well-known to those skilled in the art, or prepared in a manner analogous to routes described above for other intermediates.

### Preparation of 2-[4-(4-amino-5-oxo-7,8-dihydropyrimido[5,4-f][1,4]oxazepin-6(5H)-yl)phenyl]-2-methylpropanamide (1D)

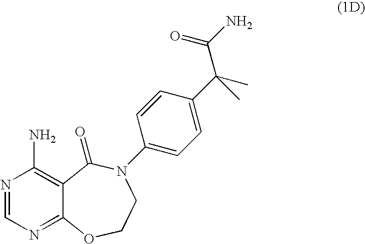

Prepared analogous to (I-1d-2) from (1D-1) to give methyl 2-(4-(4-amino-5-oxo-7,8-dihydropyrimido[5,4-f][1,4]oxazepin-6(5H)-yl)phenyl)-2-methylpropanoate which was used to form the target compound (1D) as follows:

Lithium hydroxide (40.3 mg, 1.68 mmol) and methyl 2-(4-(4-amino-5-oxo-7,8-dihydropyrimido[5,4-f][1,4]oxazepin-6(5H)-yl)phenyl)-2-methylpropanoate (200 mg, 0.561 mmol) were dissolved in a 20% solution of water in tetrahydrofuran (15.8 mL) at room temperature for 16 hours. Reaction was acidified with 1N hydrochloric acid and concentrated. Residue was diluted with a 1:1 mixture of water and 20% isopropanol in dichloromethane and stirred at room temperature for 16 hours. Precipitate was filtered off to give 2-(4-(4-amino-5-oxo-7,8-dihydropyrimido[5,4-f][1,4]oxazepin-6(5H)-yl)phenyl)-2-methylpropanoic acid (137 mg, 71%) as a white solid.

1H NMR (400 MHz; DMSO-d6) d ppm 1.46 (s, 6H) 3.90-4.00 (m, 2H) 4.48-4.61 (m, 2H) 7.23-7.42 (m, 4H) 8.14 (s, 1H)

MS (LC-MS) 343.1 (M+1)

2-(4-(4-Amino-5-oxo-7,8-dihydropyrimido[5,4-f][1,4]oxazepin-6(5H)-yl)phenyl)-2-methylpropanoic acid (31 mg, 0.091 mmol) in dimethylformamide (0.9 mL). Diisopropylethylamine (0.063 ml, 0.364 mmol) and benzotriazole-1-yl-oxy-tris-(dimethylamino)-phosphonium hexafluorophosphate (48.2 mg, 0.109 mmol) added followed by 4-methoxybenzylamine (0.012 ml, 0.091 mmol) and stirred at room temperature for 16 hours. Reaction diluted with water and extracted with ethyl acetate. Organic washed with brine then dried over sodium sulfate and concentrated. Crude product purified on silica gel eluting with 5% methanol in dichloromethane to give N-(4-methoxybenzyl)-2-(4-(4-amino-5-oxo-7,8-dihydropyrimido[5,4-f][1,4]oxazepin-6(5H)-yl)phenyl)-2-methylpropanamide (19 mg, 45%).

1H NMR (400 MHz, DMSO-d6) d ppm 1.45 (s, 6H) 3.68 (s, 3H) 3.91-3.96 (m, 2H) 4.14 (d, J=5.82 Hz, 2H) 4.51-4.59 (m, 2H) 6.81 (d, J=8.31 Hz, 2H) 7.04 (d, J=8.31 Hz, 2H) 7.26-7.36 (m, 4H) 8.14 (s, 1H)

MS (LC-MS) 462.2 (M+1)

N-(4-methoxybenzyl)-2-(4-(4-amino-5-oxo-7,8-dihydropyrimido[5,4-f][1,4]oxazepin-6(5H)-yl)phenyl)-2-methylpropanamide (19 mg, 0.041 mmol) in trifluoroacetic acid (1 mL) in a sealed tube. Heated to 50° C. for 32 hours. Reaction concentrated and diluted with saturated aqueous sodium bicarbonate and stirred at room temperature 16 hours. Aqueous decanted and residue diluted with ethyl acetate and stirred at room temperature for 1 hour. Precipitate was filtered off and dried under high vacuum to give 2-[4-(4-amino-5-oxo-7,8-dihydropyrimido[5,4-f][1,4]oxazepin-6(5H)-yl)phenyl]-2-methylpropanamide (1D) (1.6 mg, 11%) as a white solid.

1H NMR (400 MHz, DMSO-d6) d ppm 1.42 (s, 6H) 3.94 (t, J=4.57 Hz, 2H) 4.49-4.59 (m, 2H) 7.21-7.41 (m, 4H) 8.14 (s, 1H)

MN (LC-MS) 342.0 (M+1)

### Preparation of 4-amino-6-{4-[trans-4-(2-hydroxyethyl)cyclohexyl]phenyl}-7,8-dihydropyrimido[5,4-f][1,4]oxazepin-5(6H)-one (1E)

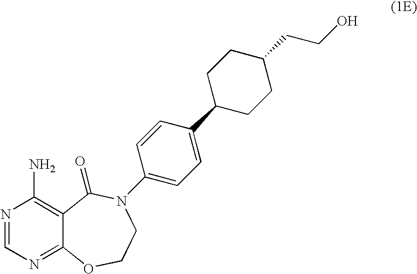

(I-1f-1) (200 mg, 0.504 mmol) in tetrahydrofuran (5 mL) was cooled to 0° C. and isopropyl chloroformate (1 mL, 1 mmol) and triethylamine (0.155 mL, 1.11 mmol) were added. The reaction mixture was warmed to room temperature for 2 hours. The reaction mixture was cooled to −78° C. and sodium borohydride (76 mg, 4 eq) in a 10% solution of methanol in tetrahydrofuran (1.65 ml) was added drop wise. Once addition was complete, reaction was allowed to warm up to room temperature for 16 hours. Water was added and reaction was concentrated to get rid of all organics. The remaining aqueous mixture was extracted with ethyl acetate (3×5 ml) and the combined organics were dried over sodium sulfate, filtered and concentrated. The residue was purified on silica gel eluting with a gradient from 0% to 10% methanol in dichloromethane to give 4-amino-6-{4-[trans-4-(2-hydroxyethyl)cyclohexyl]phenyl}-7,8-dihydropyrimido[5,4-f][1,4]oxazepin-5(6H)-one (1E) (89 mg, 46%) to give a white color solid product.

1H NMR (400 MHz, DMSO-d6) d ppm 0.84-1.10 (m, 2H) 1.22-1.35 (m, 2H) 1.36-1.48 (m, 2H) 1.61-1.91 (m, 5H) 2.27-2.60 (m, 1H) 3.32-3.53 (m, 2H) 3.91 (t, 2H) 4.27 (t, J=5.08 Hz, 1H) 4.53 (t, 2H) 7.08-7.37 (m, 4H) 7.54 (s, 2H) 8.11 (s, 1H).

ES+ 383.4 m/z.

### Preparation of 2-[4-(4-amino-5-oxo-7,8-dihydropyrimido[5,4-f][1,4]oxazepin-6(5H)-yl)phenyl]-2-methylpropanenitrile (1F)

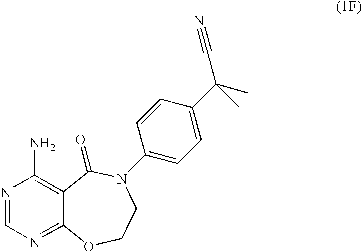

To a stirred solution of (1D) (72 mg, 0.21 mmol) in tetrahydrofuran (2.11 ml) and dimethylformamide (0.016 ml) was added oxalyl chloride (0.09 ml, 1 mmol) at room temperature and stirred for 2 hours. Saturated aqueous sodium bicarbonate was carefully added and reaction diluted with ethyl acetate. Solution was allowed to stir at room temperature for 1 hour. Precipitate was collected and dried under high vacuum to give 2-[4-(4-amino-5-oxo-7,8-dihydropyrimido[5,4-f][1,4]oxazepin-6(5H)-yl)phenyl]-2-methylpropanenitrile (1F) (23.7 mg, 34%) as a white solid.

1H NMR (400 MHz, DMSO-d6) d ppm 1.68 (s, 6H) 3.98-4.06 (m, 2H) 4.60-4.67 (m, 2H) 7.36-7.46 (m, 2H) 7.56 (d, J=8.72 Hz, 2H) 8.24 (s, 1H)

MS (LC-MS) 324.1 (M+1)

### Preparation of (8R)-4-amino-6-[4-(1-fluoro-1-methylethyl)phenyl]-8-methyl-7,8-dihydropyrimido[5,4-f][1,4]oxazepin-5(6H)-one (1L)

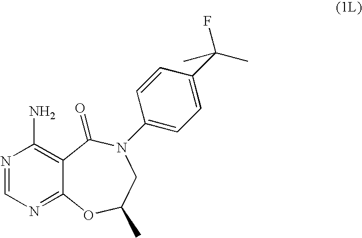

Methyl 4-[(8R)-4-amino-8-methyl-5-oxo-7,8-dihydropyrimido[5,4-f][1,4]oxazepin-6(5H)-yl]benzoate (1J) (0.201 mg, 0.612 mmol) in tetrahydrofuran (2 mL) was cooled to 0° C. Methylmagnesium bromide (1M in butyl ether, 8.57 mL) was added and stirred for 30 minutes. 1M hydrochloric acid (2.66 mL) was added and stirred for 10 minutes at 0° C. The reaction mixture was then extracted with ethyl acetate (10 ml) and the organic washed with water (2×2 ml), dried over sodium sulfate, filtered and concentrated. The crude was purified on silica gel eluting with a gradient from 20% to 75% ethyl acetate in heptane to give (8R)-4-amino-6-(4-(2-hydroxypropan-2-yl)phenyl)-8-methyl-7,8-dihydropyrimido[5,4-f][1,4]oxazepin-5(6H)-one (1A-2) (13.8 mg, 6.8%).

1H NMR (400 MHz, METHANOL-d4) d ppm 1.37 (d, J=6.44 Hz, 3H) 1.52 (s, 6H) 3.81-3.98 (m, 2H) 4.91-5.09 (m, 1H) 7.24-7.36 (m, 2H) 7.49-7.65 (m, 2H) 8.17 (s, 1H)

(8R)-4-amino-6-(4-(2-hydroxypropan-2-yl)phenyl)-8-methyl-7,8-dihydropyrimido[5,4-f][1,4]oxazepin-5(6H)-one (1A-2) (35 mg, 0.11 mmol) was dissolved in dichloromethane (4 mL) and cooled to −78° C. Deoxofluor® was added and warmed to room temperature and stirred for 34 hours. Saturated aqueous sodium bicarbonate was added and stirred for 30 minutes. Aqueous was extracted with dichloromethane and organics washed with brine, dried over magnesium sulfate, filtered and concentrated. Residue was purified on silica gel eluting with a gradient from 0% to 10% methanol in dichloromethane to give the target compound (1L) (4 mg, 10%).

1H NMR (400 MHz, CHLOROFORM-d) d ppm 1.47 (d, J=6.44 Hz, 3H) 1.67 (s, 3H) 1.73 (s, 3H) 3.80-3.96 (m, 2H) 4.89-4.99 (m, 1H) 5.64 (br. s., 1H) 7.28 (d, J=8.59 Hz, 2H) 7.43-7.51 (m, 2H) 8.00 (br. s., 1H) 8.30 (s, 1H)

### Preparation of 1-{4-[(8R)-4-amino-8-methyl-5-oxo-7,8-dihydropyrimido[5,4-f][1,4]oxazepin-6(5H)-yl]phenyl}cyclohexanecarboxamide (1AE)

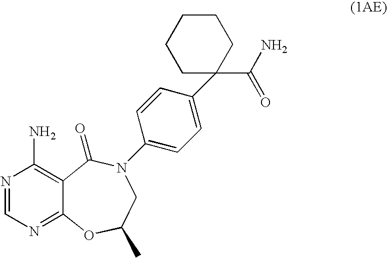

Prepared analogous to (I-1d-2) from (1AE-1) to give (8R)-methyl 1-(4-(4-amino-8-methyl-5-oxo-7,8-dihydropyrimido[5,4-f][1,4]oxazepin-6(5H)-yl)phenyl)cyclohexanecarboxylate which was used to form the target compound (1AE) as follows:

(8R)-methyl 1-(4-(4-amino-8-methyl-5-oxo-7,8-dihydropyrimido[5,4-f][1,4]oxazepin-6(5H)-yl)phenyl)cyclohexanecarboxylate (790 mg, 1.9 mmol) was dissolved in a mixture of methanol (15 mL), water (10 mL) and tetrahydrofuran (5 mL). Lithium hydroxide (810 mg, 1.9 mmol) added. The reaction mixture was then heated to 45° C. for 16 hours. Reaction mixture was cooled to room temperature and acidified by aqueous citric acid, causing a sticky solid to precipitate out. This was then extracted into ethyl acetate, dried over magnesium sulfate, filtered and concentrated. The crude product was purified on silica gel eluting with 10% methanol in ethyl acetate to give a colorless solid. Solid was then triturated with methyl tert-butyl ether to (8R)-1-(4-(4-amino-8-methyl-5-oxo-7,8-dihydropyrimido[5,4-f][1,4]oxazepin-6(5H)-yl)phenyl)cyclohexanecarboxylic acid (105 mg, 14%).

1H NMR (400 MHz, SO(CD3)2): 8.17 (s, 1H), 8.12 (s, 1H), 7.68 (br. s, 1H), 7.40 (d, J=8.7 Hz, 2H), 7.31 (d, J=8.7 Hz, 2H), 4.88-4.83 (m, 1H), 3.90-3.77 (m, 2H), 2.32 (br. s, J=12.4 Hz, 2H), 1.63-1.35 (m, 8H), 1.23 (d, J=6.4 Hz, 3H) ppm.

(8R)-1-(4-(4-amino-8-methyl-5-oxo-7,8-dihydropyrimido[5,4-f][1,4]oxazepin-6(5H)-yl)phenyl)cyclohexanecarboxylic acid (105 mg, 0.27 mmol) was dissolved in dimethylformamide (2 ml) and O-benzotriazole-N,N,N′,N′-tetramethyl-uronium-hexafluoro-phosphate (302 mg, 0.81 mmol) was added. The mixture was stirred at room temperature for 1 hour, then concentrated ammonia (aqueous) (1 ml) added and stirred for 16 hours. Reaction was concentrated and purified by prep HPLC to give the target compound (1AE) (16 mg, 15%).

1H NMR (400 MHz, SO(CD3)2): 8.17 (s, 1H), 7.40 (d, 2H), 7.28 (d, 2H), 7.06 (s, br, 1H), 6.87 (s, br, 1H), 4.85 (m, 1H), 3.83 (m, 2H), 2.32 (s, br, 2H), 1.59-1.42 (m, 8H), 1.23 (d, 3H) ppm.

### Preparation of 1-{4-[(8R)-4-amino-8-methyl-5-oxo-7,8-dihydropyrimido[5,4-f][1,4]oxazepin-6(5H)-yl]phenyl}cyclopentanecarboxamide (1AF)

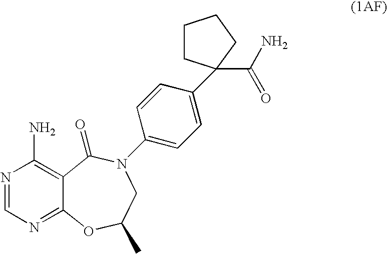

Prepared analogous to (1AE) from (1AF-1).

### Preparation of 1-{4-[(8R)-4-amino-8-methyl-5-oxo-7,8-dihydropyrimido[5,4-f][1,4]oxazepin-6(5H)-yl]phenyl}cyclobutanecarboxamide (1AH)

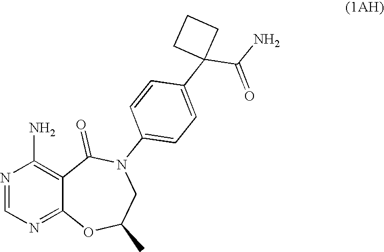

Prepared analogous to (1AE) from (1AH-1).

### Preparation of (8R)-4-amino-6-[4-(1-ethyl-1-methoxypropyl)phenyl]-8-methyl-7,8-dihydropyrimido[5,4-f][1,4]oxazepin-5(6H)-one (1AI)

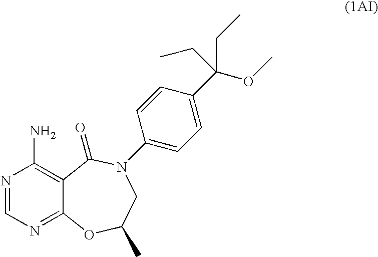

(8R)-4-Amino-6-[4-(1-ethyl-1-hydroxypropyl)phenyl]-8-methyl-7,8-dihydropyrimido[5,4-f][1,4]oxazepin-5(6H)-one (1AP) (70 mg, 0.2 mmol), hydrochloric acid in dioxane (4M solution; 1 mL) and methanol (4 mL) was stirred at room temperature for 18 hours. Reaction concentrated and diluted with ethyl acetate. Organic washed with water, dried over magnesium sulfate, filtered and concentrated. Crude purified via reverse phase chromatography using the following conditions:

MS Mode ESI+ Scan Range 160-850 daltons

Column Waters XBridge C18 19×100 mm 5 μm

Gradient from 5% to 100% of 0.05% aqueous ammonium hydroxide in acetonitrile

Prep Flow Rate 25 ml/min

(1AI) (22 mg, 30%) isolated as a white solid.

1H NMR (400 MHz, CHLOROFORM-d) d ppm 0.70 (t, 6H) 1.45 (d, 3H) 1.73-1.93 (m, 4H) 3.07 (s, 3H) 3.79-3.94 (m, 2H) 4.88-4.98 (m, 1H) 5.65 (br. s., 1H) 7.24 (d, 2H) 7.43 (d, 2H) 7.97 (br. s., 1H) 8.28 (s, 1H)

### Preparation of (8R)-4-amino-6-[4-(1-ethoxy-1-methylethyl)phenyl]-8-methyl-7,8-dihydropyrimido[5,4-f][1,4]oxazepin-5(6H)-one (1AK)

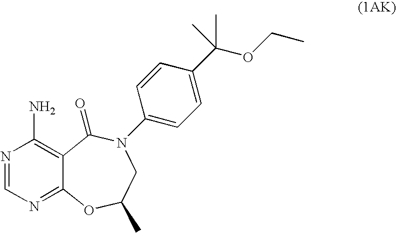

(8R)-4-amino-6-(4-(2-hydroxypropan-2-yl)phenyl)-8-methyl-7,8-dihydropyrimido[5,4-f][1,4]oxazepin-5(6H)-one (110 mg, 0.335 mmol) and 0.5M hydrochloric acid in ethanol (4 ml) was stirred at room temperature for 18 hours. Aqueous saturated sodium bicarbonate added, extracted with ethyl acetate, dried over magnesium sulfate, filtered and concentrated. Residue purified on silica gel eluting with a gradient from 0% to 7% methanol in ethyl acetate to give the target compound, (1AK) (50 mg, 42%).

1H NMR (500 MHz, CHLOROFORM-d) d ppm 1.18 (t, 3H) 1.48 (d, 3H) 1.55 (s, 6H) 3.27 (q, 2H) 3.82-3.96 (m, 2H) 4.91-5.00 (m, 1H) 5.74 (br. s., 1H) 7.26 (d, 2H) 7.51 (d, 2H) 8.01 (br. s., 1H) 8.30 (s, 1H)

### Preparation of (8R)-4-amino-6-[4-(1-methoxy-1-methylethyl)phenyl]-8-methyl-7,8-dihydropyrimido[5,4-f][1,4]oxazepin-5(6H)-one (1AL)

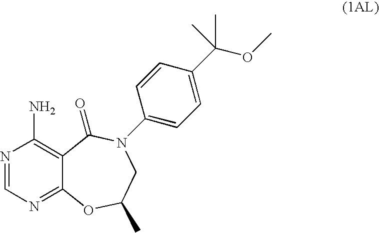

Prepared analogous to (1AI) from (8R)-4-amino-6-(4-(2-hydroxypropan-2-yl)phenyl)-8-methyl-7,8-dihydropyrimido[5,4-f][1,4]oxazepin-5(6H)-one

### Preparation of (8R)-2-(4-(4-amino-8-methyl-5-oxo-7,8-dihydropyrimido[5,4-f][1,4]oxazepin-6(5H)-yl)phenyl)acetamide (1AM)

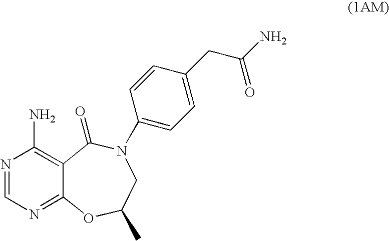

Prepared analogous to (I-1d-2) from methyl 2-(4-bromophenyl)acetate to give (8R)-methyl 2-(4-(4-amino-8-methyl-5-oxo-7,8-dihydropyrimido[5,4-f][1,4]oxazepin-6(5H)-yl)phenyl)acetate which was used to synthesize (1AM) as follows:

(8R)-methyl 2-(4-(4-amino-8-methyl-5-oxo-7,8-dihydropyrimido[5,4-f][1,4]oxazepin-6(5H)-yl)phenyl)acetate (530 mg, 1.46 mmol) and aqueous ammonium hydroxide (770 mg, 6.15 mmol) in acetonitrile (4.8 mL) were combined in a sealed tube and heated to 50° C. for 16 hours. Reaction concentrated and purified on silica gel eluting with a gradient from 5% to 20% methanol in dichloromethane. Obtained solid was then purified further via reverse phase chromatography to give the target compound (1AM) (92 mg, 19%).

### Preparation of 2-{4-[(8R)-4-amino-8-methyl-5-oxo-7,8-dihydropyrimido[5,4-f][1,4]oxazepin-6(5H)-yl]phenyl}-2-methylpropanoic acid (1AQ)

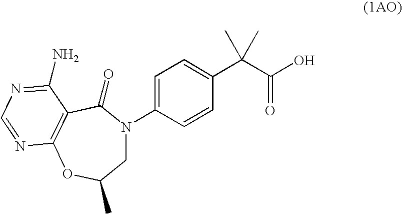

(1X) (100 mg, 0.270 mmol) was dissolved in tetrahydrofuran (2.7 mL) and water and potassium hydroxide (60.6 mg, 1.08 mmol) added. Reaction stirred at room temperature for 16 hours the heated to 50° C. for another 16 hours. Reaction concentrated to dryness and diluted with water. Aqueous was carefully acidified with 1N aqueous hydrochloric acid and solids collected to give the target compound (1AO).

1H NMR (400 MHz, DMSO-d6) d ppm 1.24 (d, J=6.44 Hz, 3H) 1.46 (s, 6H) 3.76-3.91 (m, 2H) 4.81-4.91 (m, 1H) 7.30 (d, 2H) 7.37 (d, 2H) 7.58 (br. s., 2H) 8.17 (s, 1H) 12.36 (br. s., 1H); LCMS (157-rx2) shows Desired acid at rt1.52 min, M+1=357.0, M−1=355.0.

### Preparation of 2-{4-[(8R)-4-amino-8-methyl-5-oxo-7,8-dihydropyrimido[5,4-f][1,4]oxazepin-6(5H)-yl]phenyl}-2-methylpropanamide) (1AR)

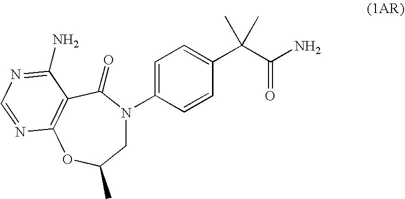

(1AO) (90 mg, 0.25 mmol) was dissolved in dimethylformamide and 1-hydroxybenzotriazole (160 mg, 1.01 mmol) and 3-[cyano(ethyl)amino]propyl-dimethylazanium chloride (100 mg, 0.506 mmol) added and stirred at room temperature for 2 hours then at 50° C. for 2 hours. Ammonia hydroxide (158 mg, 1.26 mmol) added and stirred for 2 hours at room temperature. Ethyl acetate and water were added and organic was separated, washed with brine, dried over sodium sulfate, filtered and concentrated. Crude purified on silica gel eluting with a gradient from 1% to 20% methanol in dichloromethane to give the target compound (1AR) (15 mg, 17%).

1H NMR (400 MHz, DMSO-d6) d ppm 1.24 (d, J=6.25 Hz, 3H) 1.42 (s, 6H) 3.76-3.91 (m, 2H) 4.81-4.90 (m, 1H) 6.63 (br. s., 1H) 6.89 (s, 1H) 6.94 (s, 1H) 7.28 (d, 2H) 7.36 (d, 2H) 7.57 (br. s., 1H) 8.17 (s, 1H)

M+1=356.1

TABLE 1

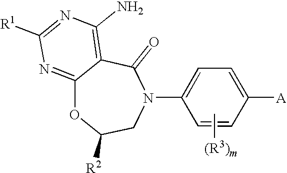

| Ex. No. | R¹ | R² | R³ | m | A | SM-1 |
|---|---|---|---|---|---|---|
| 1C | H | H | — | 0 | —C(CH₃)₃ | 4-tert-butylphenyl |
| | | | | | | trifluoromethanesulfonate |

IH NMR :(CDCI3): δ ppm 8.3 (s, IH), 7.5 (d, 2H), 7.2 (d, 2H), 4.7 (t, 2H), 4.0(t, 2H), I .3(s, 9H)
m/z = 313.5 (M + 1)

| | | | | | | |
|---|---|---|---|---|---|---|
| 1D | H | H | — | 0 | —C(CH₃)₂C(O)NH₂ | methyl 2-(4-bromophenyl)-2- |
| | | | | | | methylpropanoate (1D-1) |

1H NMR (400 MHz, DMSO-d6) δ ppm 1.42 (s, 6H) 3.94 (t, J = 4.57 Hz, 2H) 4.49-4.59 (m, 2H) 7.21-7.41 (m, 4H) 8.14 (s, 1H)
m/z = 342.0 (M + 1)

| | | | | | | |
|---|---|---|---|---|---|---|
| 1E | H | H | — | 0 | 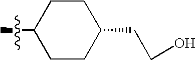 | methyl[trans-4-[4- [[(trifluoromethyl)- sulfonyl]oxy]phenyl]- cyclohexyl]acetate |

1H NMR (400 MHz, DMSO-d6) δ ppm 0.84-1.10 (m, 2H) 1.22-1.35 (m, 2H) 1.36-1.48 (m, 2H) 1.61-1.91
(m, 5H) 2.27-2.60 (m, 1H) 3.32-3.53 (m, 2H) 3.91 (t, 2H) 4.27 (t, J = 5.08 Hz, 1H) 4.53 (t, 2H) 7.08-7.37
(m, 4H) 7.54 (s, 2H) 8.11 (s, 1H).
m/z = 383.4 (M + 1)

| | | | | | | |
|---|---|---|---|---|---|---|
| 1F | H | H | — | 0 | —C(CH₃)₂CN | methyl 2-(4-bromophenyl)-2- |
| | | | | | | methylpropanoate (1D-1) |

1H NMR (400 MHz, DMSO-d6) δ ppm 1.68 (s, 6H) 3.98-4.06 (m, 2H) 4.60-4.67 (m, 2H)
7.36-7.46 (m, 2H) 7.56 (d, J = 8.72 Hz, 2H) 8.24 (s, 1H)
m/z = 324.1 (M + 1)

| | | | | | | |
|---|---|---|---|---|---|---|
| 1G | H | H | — | 0 | —CH₃ | 1-chloro-4-methylbenzene |

1H NMR (400 MHz, CHLOROFORM-d) δ ppm 2.34 (s, 3H) 3.93-3.99 (m, 2H) 4.64-4.67 (m, 2H)
7.10-7.15 (m, 2H) 7.20-7.25 (m, 2H) 8.24 (s, 1H)
m/z = 271.3 (M + 1)

| | | | | | | |
|---|---|---|---|---|---|---|
| 1H | H | H | — | 0 | —CH(CH₃)₂ | 1-bromo-4-isoproplbenzene |

1H NMR (400 MHz, METHANOL-d4) δ ppm 1.25 (d, J = 7.03 Hz, 6H) 2.87-2.98 (m, 1H) 4.00-4.04 (m, 2H)
4.67-4.71 (m, 2H) 7.23-7.27 (m, 2H) 7.30-7.34
m/z = 299.2 (M + 1)

| | | | | | | |
|---|---|---|---|---|---|---|
| 1I | H | CH₃ | — | 0 | —C(CH₃)₃ | 4-tert-butylphenyl |
| | | | | | | trifluoromethanesulfonate |

1H NMR (400 MHz, CHLOROFORM-d) δ ppm 1.32 (s, 9H) 1.45 (d, J = 6.23 Hz, 3H) 3.66-3.95 (m, 2H) 4.79-5.05
(m, 1H) 7.20 (d, J = 8.72 Hz, 2H) 7.46 (d, J = 8.72 Hz, 2H) 8.29 (s, 1H)
m/z = 327.1 (M + 1)

| | | | | | | |
|---|---|---|---|---|---|---|
| 1J | H | CH₃ | — | 0 | —C(O)OCH₃ | methyl 4-bromobenzoate |

1H NMR (400 MHz, CHLOROFORM-d) δ ppm 1.40 (d, 3H) 3.81-3.90 (m, 5H) 4.84-4.93 (m, 1H) 6.15
(br. s., 1H) 7.31-7.38 (m, 2H) 7.32-7.38 (m, 2H) 7.87 (br. s., 1H) 8.04-8.12 (m, 2H) 8.20-8.28 (m, 1H)
m/z = 329.3 (M + 1)

| | | | | | | |
|---|---|---|---|---|---|---|
| 1K | H | CH₃ | — | 0 | —CH(CH₃)₂ | 1-bromo-4-isopropylbenzene |

1H NMR (500 MHz, DMSO-d6) δ ppm 1.22 (d, J = 7.07 Hz, 6H) 1.27 (d, J = 6.34 Hz, 3H)
2.82-3.02 (m, 1H) 3.77-3.91 (m, 2H) 4.79-4.97 (m, 1H) 7.29 (q, 4H) 8.20 (s, 1H)
m/z = 313.5 (M + 1)

| | | | | | | |
|---|---|---|---|---|---|---|
| 1L | H | CH₃ | — | 0 | —CF(CH₃)₂ | methyl 4-bromobenzoate |

1H NMR (400 MHz, CHLOROFORM-d) δppm 1.47 (d, J = 6.44 Hz, 3H) 1.67 (s, 3H) 1.73 (s, 3H) 3.80-3.96 (m, 2H) 4.89-4.99
(m, 1H) 5.64 (br. S., 1H) 7.28 (d, J = 8.59 Hz, 2H) 7.43-7.51 (m, 2H) 8.00 (br. s., 1H) 8.30 (s, 1H).
m/z = 331.4 (M + 1)

| | | | | | | |
|---|---|---|---|---|---|---|
| 1M | H | CH₃ | — | 0 | —CH₂CH₃ | 1-bromo-4-ethylbenzene |

1H NMR (400 MHz, CHLOROFORM-d) δ ppm 1.26 (t, J = 7.61 Hz, 3H) 1.47 (d, J = 6.44 Hz, 3H) 2.68 (q, J = 7.61 Hz, 2H)
3.71-4.03 (m, 2H) 4.85-5.01 (m, 1H) 5.60 (br. s., 1H) 7.19 (d, 2H) 7.28 (d, 2H) 8.04 (br. s., 1H) 8.30 (s, 1H)
m/z = 299.3 (M + 1)

| | | | | | | |
|---|---|---|---|---|---|---|
| | | | | | | 4-tert-butylphenyl |
| 1N | —OCH₃ | CH₃ | — | 0 | —C(CH₃)₃ | trifluoromethanesulfonate |

1H NMR (400 MHz, CHLOROFORM-d) δ ppm 1.31 (s, 9H) 1.45 (d, J = 6.44 Hz, 3H)
3.84-3.87 (m, 2H) 3.92 (s, 3H) 4.85-4.91 (m, 1H) 7.16-7.20 (m, 2H) 7.41-7.45 (m, 2H)
m/z = 357.4 (M + 1)

| | | | | | | |
|---|---|---|---|---|---|---|
| 1O | H | CH₃ | — | 0 | 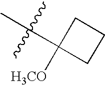 | 1-(4-bromophenyl) cyclobutanol (1O-1) |

1H NMR (400 MHz, CHLOROFORM-d) δ ppm 1.48 (d, J = 6.44 Hz, 3H) 1.65-1.75 (m, 1H) 1.90- 2.05 (m, 1H)
2.37-2.42 (m, 4H) 2.97 (s, 3H) 3.84-3.97 (m, 2H) 4.90-5.00 (m, 1H) 5.64 (br. s., 1H) 7.27-7.32
(m, 2H) 7.46-7.57 (m, 2H) 8.00 (br. s., 1H) 8.30 (s, 1H)
m/z = 355.4 (M + 1)

| | | | | | | |
|---|---|---|---|---|---|---|
| 1P | H | CH₃ | — | 0 | —CH₃ | 1-chloro-4-methylbenzene |

1H NMR (400 MHz, DMSO-d6) δ ppm 1.23 (d, J = 6.44 Hz, 3H) 2.29 (s, 3H) 3.69-3.89
(m, 2H) 4.69-4.95 (m, 1H) 7.11-7.27 (m, 4H) 7.36 (br. s., 1H) 7.55 (br. s., 1H) 8.17 (s, 1H)
m/z = 285.3 (M + 1)

| | | | | | | |
|---|---|---|---|---|---|---|
| 1Q | H | CH₃ | — | 0 | 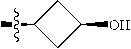 | (3-(4-bromophenyl) cyclobutoxy)(tert-butyl) dimethylsilane (1Q-1) |

1H NMR (400 MHz, CHLOROFORM-d) δ ppm 1.44 (d, J = 6.44 Hz, 3H) 1.97-2.07 (m, 2H) 2.72- 2.81
(m, 2H) 2.91-3.02 (m, 1H) 3.81-3.91 (m, 2H) 4.23-4.33 (m, 1H) 4.87-4.95 (m, 1H) 7.17-7.22 (m, 2H) 7.27-7.32 (m, 2H) 8.27 (s, 1H)
m/z = 341.3 (M + 1)

| | | | | | | |
|---|---|---|---|---|---|---|
| | | | | | | 2,2,2-trifluoro-1-(4- |
| 1R | H | CH₃ | — | 0 | —CH(OH)CF₃ | iodophenyl)ethanol (1R-1) |

1H NMR (400 MHz, acetone) δ ppm 1.39 (d, J = 6.44 Hz, 3H) 3.93-4.06 (m, 2H) 4.96-5.01 (m, 1H)
5.28 (q, J = 7.29 Hz, 1H) 7.47 (d, J = 8.40 Hz, 2H) 7.63 (d, J = 8.40 Hz, 2H) 8.14 (s, 1H) 8.20 (s, 1H).
m/z = 367.2 (M + 1)

| | | | | | | |
|---|---|---|---|---|---|---|
| 1S | H | CH₃ | — | 1 | taken together with | 5-bromo-2,3-dihydro- |
| | | | | | R³ forms a fused | 1H-indene |
| | | | | | cyclopentyl ring | |

1H NMR (CDCl3, 400 MHz): δ ppm 8.28 (s, 1H), 8.12 (bs, 1H, 7.27 (d, 1H), 7.25 (s, 1H), 7.01
(d, 1H), 5.61 (bs, 1H), 4.90-4.97 (m, 1H), 3.78-3.90 (m, 2H), 2.91-2.97 (m, 4H), 2.05-2.12 (m, 2H), 1.46 (d, 3H)
m/z = 311.4 (M + 1)

| | | | | | | |
|---|---|---|---|---|---|---|
| 1T | H | CH₃ | — | 0 | 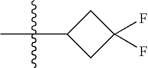 | 1-bromo-4-(3,3- difluorocyclobutyl)benzene |

1H NMR (CDCl₃, 400 MHz): δ ppm 8.29 (s, 1H), 8.00 (bs, 1H), 7.35 (d, 2H), 7.27 (d, 2H), 5.61 (bs,
1H), 4.90-4.97 (m, 1H), 3.80-3.92 (m, 2H), 3.38-3.48 (m, 1H), 2.98-3.09 (m, 2H), 2.60-2.75 (m, 2H), 1.59 (d, 3H)
m/z = 361.3 (M + 1)

| | | | | | | |
|---|---|---|---|---|---|---|
| | | | | | | 1-bromo-4- |
| 1U | H | CH₃ | — | 0 | cyclopropyl | cyclopropylbenzene |

1H NMR (400 MHz, CHLOROFORM-d) δ ppm 0.66-0.71 (m, 2H) 0.95-1.00 (m, 2H) 1.44 (d, J =
6.44 Hz, 3H) 1.86-1.94 (m, 1H) 3.76-3.90 (m, 2H) 4.87-4.95 (m, 1H) 7.14 (d, J = 1.17 Hz, 4H) 8.28 (s, 1H)
m/z = 311.3 (M + 1)

| | | | | | | |
|---|---|---|---|---|---|---|
| 1V | H | CH₃ | — | 0 | —C(CF₃)₂OH | tert-butyl(1,1,1,3,3,3- |
| | | | | | | hexafluoro-2-(4- |
| | | | | | | iodophenyl)propan-2- |
| | | | | | | yloxy)dimethylsilane (1V-1) |

1H NMR (400 MHz, CHLOROFORM-d) δ ppm 8.25 (s, 1H), 7.92 (bs, 1H), 7.81 (d, J = 8.6, 2H),
7.37 (d, J = 8.6, 2H), 5.70 (bs, 1H), 4.97-4.88 (m, 1H), 3.93-3.81 (m, 2H), 1.63 (bs, 1H), 1.45 (d, J = 6.5, 3H).
m/z = 437.1 (M + 1)

| | | | | | | |
|---|---|---|---|---|---|---|
| 1W | H | CH₃ | — | 0 | —CH(OH)CH₃ | (1-(4-bromophenyl)ethoxy) |
| | | | | | | (tert-butyl)dimethylsilane (1W-1) |

1H NMR (400 MHz, CHLOROFORM-d) δ ppm 8.24 (s, 1H), 7.90 (bs, 1H), 7.44 (d, J = 8.7, 2H),
7.21 (d, J = 8.7, 2H), 5.63 (bs, 1H), 4.93-4.83 (m, 2H), 3.90-3.77 (m, 2H), 2.81 (bs, 1H), 1.48 (d, J = 6.5, 3H), 1.41 (d, J = 6.5, 3H).
m/z = 315.2 (M + 1)

| | | | | | | |
|---|---|---|---|---|---|---|
| 1X | H | CH₃ | — | 0 | 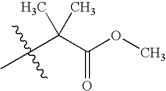 | methyl 2-(4-bromophenyl)-2- methylporpanoate (1D-1) |

1H NMR (400 MHz, CHLOROFORM-d) δ ppm 8.28 (s, 1H), 8.09 (bs, 1H), 7.41 (d, J = 8.7, 2H),
7.23 (d, J = 8.7, 2H), 5.88 (bs, 1H), 5.97-5.89 (m, 1H), 4.92-4.80 (m, 2H), 3.65 (s, 3H), 1.57 (s, 6H), 1.45 (d, J = 6.5, 3H).
m/z = 371.0 (M + 1)

| | | | | | | |
|---|---|---|---|---|---|---|
| 1Y | H | CH₃ | — | 0 | —CH₂CH(CH₃)₂ | 1-iodo-4-isobutylbenzene (1Y-1) |

1H NMR (400 MHz, CHLOROFORM-d) δ ppm 0.91 (d, J = 6.64 Hz, 6H) 1.45 (d, J = 6.44 Hz, 3H)
1.80-1.94 (m, 1H) 2.48 (d, J = 7.22 Hz, 2H) 3.81-3.89 (m, 2H) 4.87-4.98 (m, 1H) 7.14-7.23 (m, 4H) 8.28 (s, 1H)
m/z = 327.2 (M + 1)

| | | | | | | |
|---|---|---|---|---|---|---|
| 1Z | H | CH₃ | — | 0 | —C(CH₃)₂CF₃ | 4-(1,1,1-trifluoro-2- |
| | | | | | | methylpropan-2-yl)phenol 1Z-1) |

1H NMR (500 MHz, CHLOROFORM-d) δ ppm 1.49 (d, J = 6.34 Hz, 3H) 1.61 (s, 6H) 3.84-3.90 (m, 1H)
3.90-3.96 (m, 1H) 4.96 (m, J = 6.62, 6.62, 6.62, 6.62, 2.56 Hz, 1H) 5.71 (br. s., 1H) 7.31
(d, J = 8.54 Hz, 2H) 7.60 (d, J = 8.54 Hz, 2H) 8.00 (br. s., 1H)
m/z = 381.2 (M + 1)

| | | | | | | |
|---|---|---|---|---|---|---|
| 1AA | H | CH₃ | — | 0 | —C(CH₃)₂CH₂OCH₃ | 1-bromo-4-(1-methoxy-2- |
| | | | | | | methylpropan-2-yl) |
| | | | | | | benzene (1AA-1) |

1H NMR (400 MHz, CHLOROFORM-d) δ ppm 1.32 (s, 6H) 1.46 (d, J = 6.44 Hz, 3H) 3.31 (s, 3H) 3.40 (s, 2H)
3.80-3.95 (m, 2H) 4.93 (dddd, 1H) 6.50 (br. s., 1H) 7.19 (d, J = 8.78 Hz, 2H) 7.45 (d, J = 8.78 Hz, 2H) 8.23 (br. s., 1H) 8.28 (s, 1H)
m/z = 357.1 (M + 1)

| | | | | | | |
|---|---|---|---|---|---|---|
| 1AB | H | CH₃ | Cl | 1 | Cl | 4-bromo-1,2-dichlorobenzene |

1H NMR (CHLOROFORM-d) Shift: 8.23 (s, 1H), 7.83 (s, 1H), 7.46 (d, J = 8.6 Hz, 1H), 7.38 (d, J = 2.5 Hz,
1H), 7.12 (dd, J = 8.6, 2.5 Hz, 1H), 6.17 (s, 1H), 4.86 (td, J = 6.5, 2.9 Hz, 1H), 3.72-3.87 (m, 2H), 1.41 (d, J = 6.6 Hz, 3H)
m/z = 338.9 (M + 1)

| | | | | | | |
|---|---|---|---|---|---|---|
| 1AC | H | CH₃ | — | 0 | 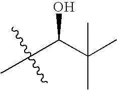 | (1-(4-bromophenyl)-2,2- dimethylpropoxy)tert- butyl)dimethylsilane (1AC-1) |

1H NMR (CHLOROFORM-d) δ ppm: 8.25 (s, 1H), 7.93 (br. s., 1H), 7.39 (d, J = 8.4 Hz, 2H),
7.21 (d, J = 8.6 Hz, 2H), 5.58 (br. s., 1H), 4.86-4.96 (m, 1H), 4.41 (s, 1H), 3.77-3.92 (m, 2H), 2.29 (s, 1H), 1.44 (d, J = 6.6 Hz, 3H), 0.93 (s, 9H)
m/z = 357.1 (M + 1)

| | | | | | | |
|---|---|---|---|---|---|---|
| 1AD | H | CH₃ | — | 0 | 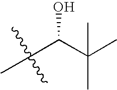 | (1-(4-bromophenyl)-2,2- dimethylpropoxy)(tert- butyl)dimethylsilane (1AC-1) |

1H NMR (CHLOROFORM-d) δ ppm: 8.22 (s, 1H), 7.89 (br. s., 1H), 7.38 (d, J = 8.4 Hz, 2H), 7.20 (d, J = 8.6 Hz,
2H), 5.62 (br. s., 1H), 4.85-4.95 (m, 1H), 4.40 (d, J = 2.9 Hz, 1H), 3.76-3.91 (m, 2H), 2.50 (d, J = 2.9 Hz, 1H), 1.43 (d, J = 6.4 Hz, 3H), 0.
m/z = 357.1 (M + 1)

| | | | | | | |
|---|---|---|---|---|---|---|
| 1AE | H | CH₃ | — | 0 | 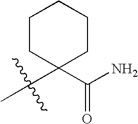 | Methyl 1-(4-bromophenyl) cyclohexanecarboxylate (1AE-1) |

1H NMR (400 MHz, DMSO-d6: δ ppm 8.17 (s, 1H), 7.40 (d, 2H), 7.28 (d, 2H), 7.06 (s, br, 1H), 6.87
(s, br, 1H), 4.85 (m, 1H), 3.83 (m, 2H), 2.32 (s, br, 2H), 1.59-1.42 (m, 8H), 1.23 (d, 3H) ppm.
m/z = 396 (M + 1)

| | | | | | | |
|---|---|---|---|---|---|---|
| 1AF | H | CH₃ | — | 0 | 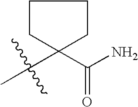 | methyl 1-(4-bromophenyl) cylcopentanecarboxylate (1AF-1) |

1H NMR (400 MHz, DMSO-d6) δ ppm 8.17 (s, 1H), 7.60 (br. s, 1H), 7.36 (d, 2H), 7.28 (d, 2H), 7.06 (br. s, 1H),
6.80 (br. s, 1H), 4.85 (m, 1H), 3.83 (m, 2H), 2.53 (m, 2H), 1.70 (m, 2H), 1.64-1.50 (m, 4H), 1.23 (d, 3H).
m/z = 382.2 (M + 1)

| | | | | | | |
|---|---|---|---|---|---|---|
| 1AG | H | CH₃ | — | 0 | —CH₂C(CH₃)₂OCH₃ | 1-bromo-4-(2-methoxy-2- |
| | | | | | | methylpropyl)benzene (1AG-1) |

1H NMR (400 MHz, CHLOROFORM-d) δ ppm 1.14 (s, 6H) 1.44 (d, J = 6.44 Hz, 3H) 2.77 (s, 2H) 3.26 (s, 3H)
3.76-3.93 (m, 2H) 4.86-4.97 (m, 1H) 5.66 (br. s., 1H) 7.17 (d, 2H) 7.26 (d, 2H) 7.99 (br. s., 1H) 8.27 (s, 1H)
m/z = 357.1 (M + 1)

| | | | | | | |
|---|---|---|---|---|---|---|
| 1AH | H | CH₃ | — | 0 | 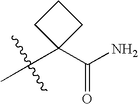 | methyl 1-(4-bromophenyl) cyclobutanecarboxylate (1AH-1) |

1H NMR (400 MHz, DMSO-d6) 8.17 (s, 1H), 7.62 (s, 1H), 7.26 (d, 2H), 7.22 (d, 2H), 4.87 (m, 1H), 3.84-3.82
(m, 2H), 3.47 (dd, 1H), 3.30-3.25 (m, 2H), 2.05 (m, 1H), 1.78-1.60 (m, 4H), 1.24 (d, 3H).
m/z = 368.0 (M + 1)

| | | | | | | |
|---|---|---|---|---|---|---|
| 1AI | H | CH₃ | — | 0 | 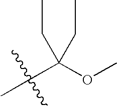 | 2-(4-bromophenyl)-2- ethylbutan-1-ol (1AI-1) |

1H NMR (400 MHz, CHLOROFORM-d) δ ppm 0.70 (t, 6H) 1.45 (d, 3H) 1.73-1.93 (m, 4H) 3.07
(s, 3H) 3.79-3.94 (m, 2H) 4.88-4.98 (m, 1H) 5.65 (br. s., 1H) 7.24 (d, 2H) 7.43 (d, 2H) 7.97 (br. s., 1H) 8.28 (s, 1H)
m/z = 371.0 (M + 1)

| | | | | | | |
|---|---|---|---|---|---|---|
| 1AJ | H | CH₃ | — | 0 | 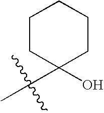 | 1-(4-bromophenyl) cyclohexanol (1AJ-1) |

1H NMR (400 MHz, CHLOROFORM-d) δ ppm 1.20-1.36 (m, 1H) 1.45 (d, 3H) 1.58-1.87 (m, 10H) 3.78-3.94 (m, 2H)
4.87-4.96 (m, 1H) 5.59 (s, 1H) 7.24 (d, 2H) 7.58 (d, 2H) 7.97 (s, 1H) 8.27 (s, 1H)
m/z = 369.1 (M + 1)

| | | | | | | |
|---|---|---|---|---|---|---|
| 1AK | H | CH₃ | — | 0 | 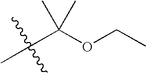 | 2-(4-bromophenyl) propan-2-ol (1AK-1) |

1H NMR (500 MHz, CHLOROFORM-d) δ ppm 1.18 (t, 3H) 1.48 (d, 3H) 1.55 (s, 6H) 3.27 (q, 2H) 3.82-3.96 (m, 2H)
4.91-5.00 (m, 1H) 5.74 (br. s., 1H) 7.26 (d, 2H) 7.51 (d, 2H) 8.01 (br. s., 1H) 8.30 (s, 1H)
m/z = 357.1 (M + 1)

| | | | | | | |
|---|---|---|---|---|---|---|
| 1AL | H | CH₃ | — | 0 | 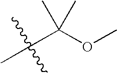 | 2-(4-bromophenyl) propan-2-ol (1AK-1) |

1H NMR (400 MHz, CHLOROFORM-d) δ ppm 1.45 (d, 3H) 1.51 (s, 6H) 3.09 (s, 3H) 3.78-3.93 (m, 2H) 4.88-4.97
(m, 1H) 5.74 (br. s., 1H) 7.24 (d, 2H) 7.47 (d, 2H) 7.98 (br. s., 1H) 8.27 (s, 1H)
m/z = 343.1 (M + 1)

| | | | | | | |
|---|---|---|---|---|---|---|
| 1AM | H | CH₃ | — | 0 | 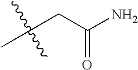 | methyl 2-(4- bromophenyl)acetate |

1H NMR (400 MHz, DMSO-d6) δ ppm 1.22 (d, J = 6.25 Hz, 3H) 3.37 (d, 2H) 3.76-3.91 (m, 2H) 4.81-4.90
(m, 1H) 6.63 (br. s., 1H) 6.89 (s, 1H) 6.94 (s, 1H) 7.28 (d, 2H) 7.36 (d, 2H) 7.57 (br. s., 1H) 8.17 (s, 1H)
m/z = 328 (M + 1)

| | | | | | | |
|---|---|---|---|---|---|---|
| 1AN | H | CH₃ | — | 0 | —CH₂C(CH₃)₂OH | 1-bromo-4-(2-methoxy-2- |
| | | | | | | methylpropyl)benzene (1AG-1) |

1H NMR (400 MHz, CHLOROFORM-d) δ ppm 1.25 (s, 6H) 1.45 (d, J = 6.44 Hz, 3H) 2.39 (br. s., 1H) 2.78
(s, 2H) 3.78-3.92 (m, 2H) 4.87-4.97 (m, 1H) 5.71 (br. s., 1H) 7.21 (d, 2H) 7.31 (d, 2H) 8.00 (br. s., 1H) 8.25 (s, 1H)
m/z = 343.0 (M + 1)

| | | | | | | |
|---|---|---|---|---|---|---|
| 1AO | H | CH₃ | — | 0 | 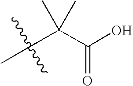 | methyl 2-(4-bromophenyl)-2- methylpropanoate (1D-1) |

1H NMR (400 MHz, DMSO-d6) δ ppm 1.24 (d, J = 6.44 Hz, 3H) 1.46 (s, 6H) 3.76-3.91 (m, 2H) 4.81-4.91
(m, 1H) 7.30 (d, 2H) 7.37 (d, 2H) 7.58 (br. s., 2H) 8.17 (s, 1H) 12.36 (br. s., 1H)
m/z = 357.0 (M + 1)

| | | | | | | |
|---|---|---|---|---|---|---|
| 1AP | H | CH₃ | — | 0 | 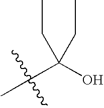 | 2-(4-bromophenyl)-2- ethylburan-1-ol (1AI-1) |

1H NMR (500 MHz, CHLOROFORM-d) δ ppm 0.81 (t, 6H) 1.49 (d, 3H) 1.73 (s, 1H) 1.80-1.95 (m, 4H)
3.83-3.97 (m, 2H) 4.92-5.01 (m, 1H) 5.64 (br. s., 1H) 7.27 (d, 2H) 7.49 (d, 2H) 8.01 (br. s., 1H) 8.31 (s, 1H)
m/z = 357.1 (M + 1)

| | | | | | | |
|---|---|---|---|---|---|---|
| 1AQ | H | CH₃ | — | 0 | 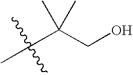 | methyl 2-(4-bromophenyl)-2- methylpropanoate (1D-1) |

1H NMR (400 MHz, DMSO-d6) δ ppm 1.20 (s, 6H) 1.24 (d, J = 6.44 Hz, 3H) 3.40 (d, J = 5.27 Hz, 2H) 3.75-3.89 (m, 2H) 4.68
(t, J = 5.27 Hz, 1H) 4.82-4.90 (m, 1H) 7.24 (d, J = 8.78 Hz, 2H) 7.36 (br. s., 1H) 7.39 (d, J = 8.78 Hz, 2H) 7.56 (br. s., 1H) 8.17
m/z = 343.0 (M + 1)

| | | | | | | |
|---|---|---|---|---|---|---|
| 1AR | H | CH₃ | — | 0 | 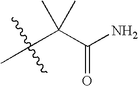 | methyl 2-(4-bromophenyl)-2- methylpropanoate (1D-1) |

1H NMR (400 MHz, DMSO-d6) δ ppm 1.24 (d, J = 6.25 Hz, 3H) 1.42 (s, 6H) 3.76-3.91 (m, 2H) 4.81-4.90
(m, 1H) 6.63 (br. s., 1H) 6.89 (s, 1H) 6.94 (s, 1H) 7.28 (d, 2H) 7.36 (d, 2H) 7.57 (br. s., 1H) 8.17 (s, 1H)
m/z = 356.1 (M + 1)

The compounds listed in Table 1A below were prepared using procedures analogous to those described above for the synthesis of 4-amino-6-{4-[trans-4-(2-hydroxy-2-methylpropyl)cyclohexyl]phenyl}-7,8-dihydropyrimido[5,4-f][1,4]oxazepin-5(6H)-one (1A) using the appropriate starting materials which are available commercially, prepared using preparations well-known to those skilled in the art, or prepared in a manner analogous to routes described above for other intermediates.

TABLE 1A

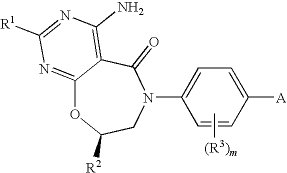

| Ex. No. | R¹ | R² | R³ | m | A | SM-1 |
|---|---|---|---|---|---|---|
| 1A-1 | H | H | — | 0 | 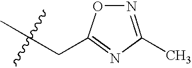 | methyl 3-(trans-4-(4-(trifluoro- methylsulfonyloxy)phenyl)- cyclohexyl)propanoate |

1H NMR (400 MHz, CHLOROFORM-d) δ ppm 0.98-1.12 (m, 2H) 1.15-1.20
(m, 6H) 1.16-1.22 (m, 1H) 1.23-1.31 (m, 2H) 1.34-1.51 (m, 4H) 1.58-1.68 (m, 1H)
1.78-1.93 (m, 4H) 2.40-2.51 (m, 1H) 3.90-4.03 (m, 2H) 4.62-4.68 (m, 2H) 5.60 (br.
s., 1H) 7.12-7.31 (m, 4H) 8.00-8.23 (m, 1H) 8.24 (s, 1H).

| m/z = 425.1 (M + 1) | | | | | | |
|---|---|---|---|---|---|---|
| 1A-2 | H | CH₃ | — | 0 | —C(CH₃)₂OH | methyl 4-(trifluoromethyl- |
| | | | | | | sulfonyloxy)benzoate |

1H NMR (400 MHz, METHANOL-d4) δ ppm 1.37 (d, J = 6.44 Hz, 3H) 1.52
(s, 6H) 3.81-3.98 (m, 2H) 4.91-5.09 (m, 1H) 7.24-7.36 (m, 2H) 7.49-7.65 (m, 2H) 8.17 (s, 1H).
m/z = 329.4 (M + 1)

### Example 2

### Preparation of 4-Amino-6-(4-{trans-4-[(3-methyl-1,2,4-oxadiazol-5-yl)methyl]cyclohexyl}phenyl)-7,8-dihydropyrimido[5,4-f][1,4]oxazepin-5(6H)-one (2A)

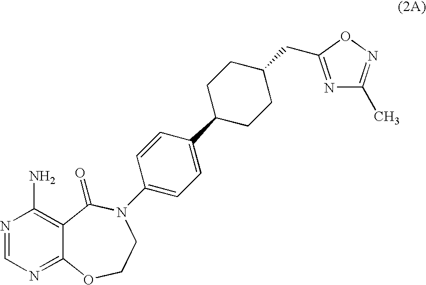

To an ice cooled, stirred mixture of {trans-4-[4-(4-amino-5-oxo-7,8-dihydropyrimido[5,4-f][1,4]oxazepin-6(5H)-yl)phenyl]cyclohexyl}acetic acid (I-1f-1: 75 mg, 0.19 mmol) in 1,2-dichloroethane (0.63 mL) was added oxalyl chloride (0.165 mL, 1.89 mmol) and the resulting thick slurry was stirred at room temperature for 2 hours. The mixture was concentrated in vacuo, azeotroped with toluene and the resulting solids dissolved in p-dioxane (1.5 mL), N-hydroxyacetamidine (140 mg, 1.9 mmol) added and the mixture stirred at room temperature overnight. The reaction mixture was concentrated in vacuo and chromatographed on silica gel (12 g column, 5-10% methanol:dichloromethane over 30 min) to afford 2-{4-[4-(4-amino-5-oxo-7,8-dihydro-5H-9-oxa-1,3,6-triaza-benzocyclohepten-6-yl)-phenyl]-cyclohexyl}-N-{1-[(E)-hydroxyimino]-ethyl}-acetamide, 86 mg.

To a stirred solution of 2-{4-[4-(4-amino-5-oxo-7,8-dihydro-5H-9-oxa-1,3,6-triaza-benzocyclohepten-6-yl)-phenyl]-cyclohexyl}-N-{1-[(E)-hydroxyimino]-ethyl}-acetamide (37 mg, 0.082 mmol) in dimethylformamide (1.0 mL) was heated under microwave conditions at 120° C. for 5 hours. The reaction mixture was concentrated in vacuo and chromatographed on silica gel (12 g column, 2.5-10% methanol:dichloromethane over 30 min) to afford the title compound (2A) as a white solid, 23 mg.

1H NMR (400 MHz, CHLOROFORM-d) 5 ppm 8.24 (s, 1H) 8.12 (br. s., 1H) 7.20-7.28 (m, 2H) 7.11-7.19 (m, 2H) 5.67 (br. s., 1H) 4.59-4.70 (m, 2H) 3.91-4.01 (m, 2H) 2.76 (d, 2H) 2.43-2.56 (m, 1H) 2.35 (s, 3H) 1.78-1.99 (m, 5H) 1.38-1.56 (m, 2H) 1.13-1.29 (m, 2H). m/z=435.1 (M+1).

The compounds listed in Table 2 below were prepared using procedures analogous to those described above for the synthesis of 4-Amino-6-(4-{trans-4-[(3-methyl-1,2,4-oxadiazol-5-yl)methyl]cyclohexyl}phenyl)-7,8-dihydropyrimido[5,4-f][1,4]oxazepin-5(6H)-one (2A) using the appropriate starting materials which are available commercially, prepared using preparations well-known to those skilled in the art, or prepared in a manner analogous to routes described above for other intermediates.

TABLE 2

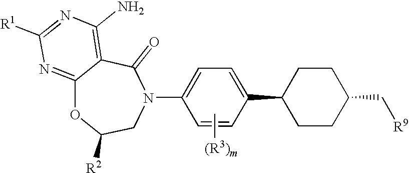

| Ex. No. | R¹ | R² | R³ | m | R⁹ | SM-1 |
|---|---|---|---|---|---|---|
| 2B | H | H | — | 0 | 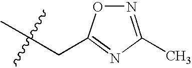 | methyl (trans-4-(4-(trifluoro- methylsulfonyloxy)phenyl)- cyclohexyl)propanoate |

1H NMR (400 MHz, CHLOROFORM-d) δ ppm 1.01-1.16 (m, 2H) 1.29-1.49 (m, 3H) 1.61-1.77
(m, 2H) 1.88 (d, J = 12.30 Hz, 4H) 2.31-2.37 (m, 3H) 2.41-2.52 (m, 1H) 2.79-2.90 (m, 2H) 3.91-4.00
(m, 2H) 4.57-4.68 (m, 2H) 5.55-5.80 (m, 1H) 7.09-7.27 (m, 4H) 8.02-8.21 (m, 1H) 8.23 (s, 1H).
m/z = 449.3 (M + 1)

| | | | | | | |
|---|---|---|---|---|---|---|
| 2C | H | CH₃ | — | 0 | 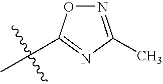 | methyl[trans-4-[4- [[(trifluoromethyl)- sulfonyl]oxy]phenyl]- cyclohexyl]acetate |

1H NMR (400 MHz, CHLOROF0RM-d) δ ppm 1.23 (br. s., 2H) 1.37-1.53 (m, 5H) 1.83-2.03 (m,
5H) 2.37 (s, 3H) 2.48-2.57 (m, 1H) 2.79 (d, J = 6.83 Hz, 2H) 3.71-3.95 (m, 2H) 4.77-5.00 (m, 1H)
5.61 (br. s., 1H) 6.38 (none, 1H) 7.14-7.22 (m, 2H) 7.19-7.29 (m, 2H) 8.00 (br. s., 1H) 8.27 (s, 1H)
m/z = not available

### Example 3

### Preparation of 4-Amino-6-(4-{trans-4-[(5-methyl-1,3,4-oxadiazol-2-yl)methyl]cyclohexyl}phenyl)-7,8-dihydropyrimido[5,4-f][1,4]oxazepin-5(6H)-one (3A)

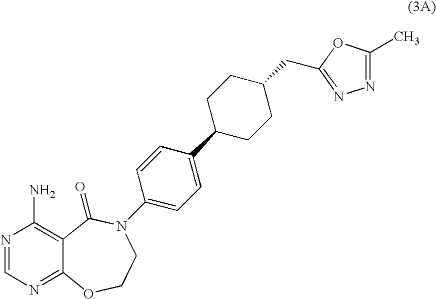

To an ice cooled, stirred mixture of {trans-4-[4-(4-amino-5-oxo-7,8-dihydropyrimido[5,4-f][1,4]oxazepin-6(5H)-yl)phenyl]cyclohexyl}acetic acid (I-1f-1: 100 mg, 0.252 mmol) in 1,2-dichloroethane (0.84 mL) was added oxalyl chloride (0.221 mL, 2.52 mmol) and the resulting thick slurry was stirred at room temperature for 2 hours. The mixture was concentrated in vacuo, azeotroped with toluene and the resulting solids dissolved in p-dioxane (1.5 mL), acetic hydrazide (192 mg, 2.52 mmol) added and the mixture stirred at room temperature for 96 hours. The reaction mixture was partitioned between dichloromethane and saturated aqueous sodium bicarbonate. The insoluble solids were filtered, washed with water and dried in vacuo to afford N-acetyl-N′-(2-{4-[4-(4-amino-5-oxo-7,8-dihydro-5H-9-oxa-1,3,6-triaza-benzocycloheptan-6-yl)phenyl]-cyclohexyl}-acetyl-hydrazide as a white solid, 83 mg.

To a stirred solution of triphenyl phosphine (23 mg, 0.021 mmol), iodine (21 mg, 0.084 mmol) and triethylamine (18 mg, 0.176 mmol) was added N-acetyl-N′-(2-{4-[4-(4-amino-5-oxo-7,8-dihydro-5H-9-oxa-1,3,6-triaza-benzocycloheptan-6-yl)phenyl]-cyclohexyl}-acetyl-hydrazide (20 mg, 0.044) and the resulting mixture was stirred at room temperature for 3.5 hours. The reaction mixture was concentrated in vacuo and chromatographed via prep HPLC (C18 column, 20-50% acetonitrile:water, 10 mL/min) to afford the title compound (3A) as a white solid, 6 mg.

1H NMR (400 MHz, CHLOROFORM-d) δ ppm 8.24 (s, 1H) 8.15 (br. s., 1H) 7.20-7.30 (m, 2H) 7.11-7.19 (m, 2H) 5.72 (br. s., 1H) 4.61-4.68 (m, 2H) 3.93-4.02 (m, 2H) 2.68-2.76 (m, 2H) 2.41-2.55 (m, 4H) 1.80-1.95 (m, 5H) 1.37-1.54 (m, 2H) 1.13-1.28 (m, 2H). m/z=435.3 (M+1).

### Example 4

### Preparation of 4-Amino-6-(4-{trans-4-[(5-methyl-1,3,4-thiadiazol-2-yl)methyl]cyclohexyl}phenyl)-7,8-dihydropyrimido[5,4-f][1,4]oxazepin-5(6H)-one (4A)

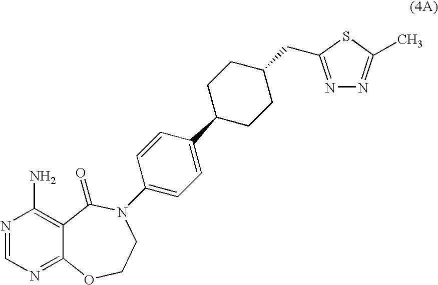

A solution of N-acetyl-N′-(2-{4-[4-(4-amino-5-oxo-7,8-dihydro-5H-9-oxa-1,3,6-triaza-benzocycloheptan-6-yl)phenyl]-cyclohexyl}-acetyl-hydrazide (from Example 3A above, 26 mg, 0.057 mmol) and Lawesson's reagent (14 mg, 0.034 mmol) in 1:1 p-dioxane:tetrahydrofuran (0.8 mL) was heated in a sealed tube at 120° C. for 18 hours. The reaction was cooled, concentrated and chromatographed via prep HPLC (C18 column, 20-50% acetonitrile:water, 10 mL/min) to afford the title compound (46 as a white solid, 2.5 mg.

1H NMR (400 MHz, METHANOL-d₄) δ ppm 8.12 (s, 1H) 7.26-7.32 (m, 2H) 7.19-7.25 (m, 2H) 4.63-4.69 (m, 2H) 3.96-4.02 (m, 2H) 2.97-3.01 (m, 2H) 2.70 (s, 3H) 2.48-2.58 (m, 1H) 1.83-1.92 (m, 5H) 1.42-1.57 (m, 2H) 1.18-1.30 (m, 2H). m/z=451.1 (M+1).

### Example 5

### Preparation of 4-amino-6-(4-{trans-4-[(4,5-dimethyl-4H-1,2,4-triazol-3-yl)methyl]cyclohexyl}phenyl)-7,8-dihydropyrimido[5,4-f][1,4]oxazepin-5(6H)-one (5A)

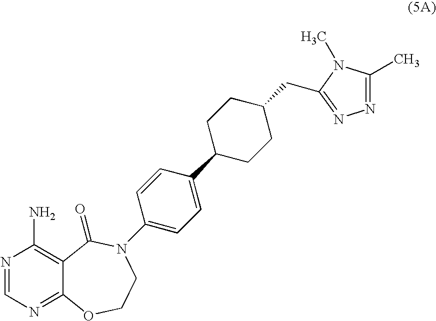

To an ice cooled, stirred mixture of {trans-4-[4-(4-amino-5-oxo-7,8-dihydropyrimido[5,4-f][1,4]oxazepin-6(5H)-yl)phenyl]cyclohexyl}acetic acid (I-1f-1: 500 mg, 0.13 mmol) in 1,2-dichloroethane (0.42 mL) was added oxalyl chloride (0.11 mL, 1.26 mmol) and the resulting thick slurry was stirred at room temperature for 2 hours. The mixture was concentrated in vacuo, azeotroped with toluene and the resulting solids dissolved in 2M methylamine in tetrahydrofuran (0.63 mL, 1.26 mmol) and stirred for 24 hours. The solids were filtered, washed with ethyl ether and dried in vacuo to afford 2-{4-[4-(4-amino-5-oxo-7,8-dihydro-5H-9-oxa-1,3,6-triaza-benzocyclohepten-6-yl)-phenyl]-cyclohexyl}-N-methylacetamide as a white solid, 50 mg.

A solution of 2-{4-[4-(4-amino-5-oxo-7,8-dihydro-5H-9-oxa-1,3,6-triaza-benzocyclohepten-6-yl)-phenyl]-cyclohexyl}-N-methylacetamide (35 mg, 0.085 mmol) and Lawesson's reagent (21 mg, 0.051 mmol) in tetrahydrofuran (0.57 mL) was heated at reflux for 3 hours. The reaction was cooled, concentrated in vacuo and chromatographed on silica gel (4 g, 2-8% methanol:dichloromethane, 30 minutes) to afford 2-{4-[4-(4-amino-5-oxo-7,8-dihydro-5H-9-oxa-1,3,6-triaza-benzocyclohepten-6-yl)-phenyl]-cyclohexyl}-N-methylthioacetamide as a yellow solid, 11 mg.

A stirred slurry of 2-{4-[4-(4-amino-5-oxo-7,8-dihydro-5H-9-oxa-1,3,6-triaza-benzocyclohepten-6-yl)-phenyl]-cyclohexyl}-N-methylthioacetamide (11 mg, 0.026 mmol), mercury oxide (6.4 mg, 0.029 mmol) and acetic hydrazide (4 mg, 0.052 mmol) in tetrahydrofuran was stirred at room temperature for 16 hours and then heated at 80° C. under microwave conditions. The reaction mixture was filtered through Celite®, washing with methanol and then chromatographed via prep HPLC (C18, 20-50% acetonitrile:water, 10 mL/min) to afford the title compound (5A) as a white solid.

1H NMR (400 MHz, CHLOROFORM-d) δ ppm 8.22 (s, 1H) 8.16 (br. s., 1H) 7.24 (d, 2H) 7.14 (d, 2H) 6.02 (br. s., 1H) 4.61-4.68 (m, 2H) 3.94-4.01 (m, 2H) 3.46 (s, 3H) 2.65 (d, 2H) 2.45-2.54 (m, 1H) 2.41 (s, 3H) 1.77-1.94 (m, 5H) 1.36-1.51 (m, 2H) 1.13-1.29 (m, 2H). m/z=448.2 (M+1).

### Preparation of 4-amino-6-(4-{trans-4-[(5-methyl-4H-1,2,4-triazol-3-yl)methyl]cyclohexyl}-phenyl)-7,8-dihydropyrimido[5,4-f][1,4]oxazepin-5(6H)-one (5B)

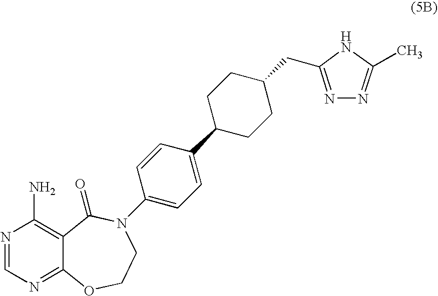

Compound 5B above can be prepared using procedures analogous to those described above for the synthesis of 4-amino-6-(4-{trans-4-[(4,5-dimethyl-4H-1,2,4-triazol-3-yl)methyl]cyclohexyl}phenyl)-7,8-dihydropyrimido[5,4-f][1,4]oxazepin-5(6H)-one (5A) with the exception that ammonia is used in place of methylamine.

1H NMR (400 MHz, CHLOROFORM-d) δ ppm 1.40-1.60 (m, 3H) 1.90 (br. s., 5H) 2.12 (d, J=6.83 Hz, 1H) 2.34-2.42 (m, 6H) 3.90-4.02 (m, 2H) 4.65 (dd, J=4.98, 3.61 Hz, 2H) 7.12-7.18 (m, 2H) 7.23-7.28 (m, 2H) 8.25 (s, 1H).

### Example 6

### Preparation of 4-amino-6-{4-[trans-4-(2-oxo-2-pyrrolidin-1-ylethyl)cyclohexyl]phenyl}-7,8-dihydropyrimido[5,4-f][1,4]oxazepin-5(6H)-one (6A)

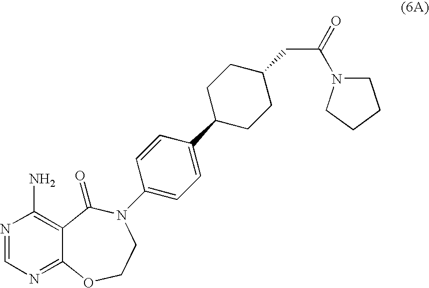

A solution of {trans-4-[4-(4-amino-5-oxo-7,8-dihydropyrimido[5,4-f][1,4]oxazepin-6(5H)-yl)phenyl]cyclohexyl}acetic acid (I-1f-1: 12 mg, 0.03 mmol), pyrrolidine (5 mg, 0.08 mmol) and O-benzotriazole-N,N,N′,N′-tetramethyl-uronium-hexafluoro-phosphate (12 mg, 0.04 mmol) in dimethylformamide (0.4 mL) was heated at 55° C. for 18 hours. Chromatography on silica gel (4 g, 1-5% methanol:dichloromethane) afforded the title compound (6A) as a white solid, 7 mg.

1H NMR (400 MHz, CHLOROFORM-d) δ ppm 8.24 (s, 1H) 8.15 (br. s., 1H) 7.10-7.35 (m, 4H) 5.63 (br. s., 1H) 4.60-4.70 (m, 2H) 3.91-4.03 (m, 2H) 3.35-3.49 (m, 4H) 2.39-2.53 (m, 1H) 2.13-2.21 (m, 2H) 1.76-1.98 (m, 9H) 1.38-1.55 (m, 2H) 1.04-1.18 (m, 2H). m/z=450.4 (M+1).

The compounds listed in Table 3 below were prepared using procedures analogous to those described above for the synthesis of 4-amino-6-{4-[trans-4-(2-oxo-2-pyrrolidin-1-ylethyl)cyclohexyl]phenyl}-7,8-dihydropyrimido[5,4-f][1,4]oxazepin-5(6H)-one (6A) using the appropriate starting materials which are available commercially, prepared using preparations well-known to those skilled in the art, or prepared in a manner analogous to routes described above for other intermediates. The structures were verified by high resolution mass spectrometry.

TABLE 3

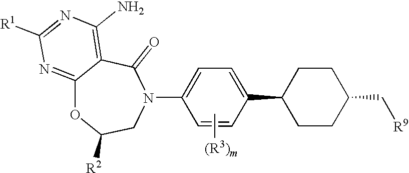

| Ex. No. | R¹ | R² | R³ | m | R⁹ |
|---|---|---|---|---|---|
| 6B | H | H | — | 0 | 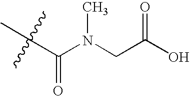 |

1H NMR (400 MHz, DMSO-d6) δ ppm 8.15 (s, 1H) 7.59 (br. s., 2H) 7.17- 7.34 (m, 4H)
4.47-4.63 (m, 2H) 3.89-4.01 (m, 2H) 3.31 (s, 3H) 3.01 (s, 2H) 2.25 (d, J = 6.64 Hz, 2H) 2.11
(d, J = 6.22 Hz, 1H) 1.67-1.91 (m, 5H) 1.34- 1.54 (m, 2H) 0.93-1.21 (m, 2H)
m/z = 468.4 (M + 1)

| | | | | | |
|---|---|---|---|---|---|
| 6C | H | H | — | 0 | 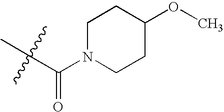 |

1H NMR (400 MHz, CHLOROFORM-d) δ ppm 8.23 (s, 1H) 8.14 (br. s., 1H) 7.11-7.30 (m, 4H)
4.60-4.68 (m, 2H) 3.88-4.00 (m, 3H) 3.61-3.74 (m, 1H) 3.15-3.46 (m, 5H) 2.39-2.52 (m, 1H) 2.23
(dd, J = 6.64, 3.73 Hz, 2H) 1.76-1.96 (m, 8H) 1.39-1.60 (m, 5H) 1.03-1.17 (m, 2H)
m/z = 494.5 (M + 1)

| | | | | | |
|---|---|---|---|---|---|
| 6D | H | H | — | 0 | 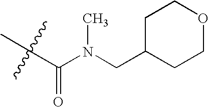 |

1H NMR (400 MHz, CHLOROFORM-d) δ ppm 8.23 (s, 1H) 8.14 (br. s., 1H) 7.09-7.31 (m, 4H)
5.64 (br. s., 1H) 4.60-4.69 (m, 3H) 3.84-4.13 (m, 4H) 3.10-3.42 (m, 4H) 2.99 (s, 3H) 2.41-2.52
(m, 1H) 2.21 (d, J = 6.64 Hz, 2H) 1.78-1.97 (m, 4H) 1.39-1.61 (m, 4H) 1.17-1.39 (m, 3H) 1.01-1.17 (m, 2H)
m/z = 508.5 (M + 1)

| | | | | | |
|---|---|---|---|---|---|
| 6E | H | H | — | 0 | 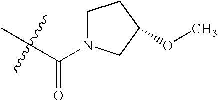 |

m/z = 480.5 (M + 1)

| | | | | | |
|---|---|---|---|---|---|
| 6F | H | H | — | 0 | 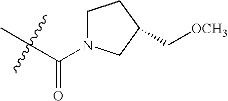 |

m/z = 494.5 (M + 1)

| | | | | | |
|---|---|---|---|---|---|
| 6G | H | H | — | 0 | 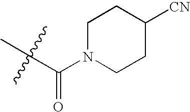 |

m/z = 489.5 (M + 1)

| | | | | | |
|---|---|---|---|---|---|
| 6H | H | H | — | 0 | 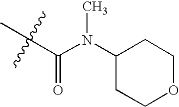 |

m/z = 494.5 (M + 1)

| | | | | | |
|---|---|---|---|---|---|
| 6I | H | H | — | 0 | 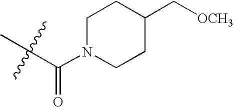 |
| m/z = 508.5 (M + 1) | | | | | |
| 6J | H | H | — | 0 | 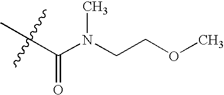 |

1H NMR (400 MHz, CHLOROFORM-d) δ ppm 8.28 (s, 1H) 8.19 (br. s., 1H) 7.14-7.33 (m, 4H) 4.64-4.74
(m, 2H) 3.97-4.03 (m, 2H) 3.52-3.60 (m, 2H) 3.50 (s, 1H) 3.30-3.39 (m, 3H) 3.08 (s, 3H) 2.97 (s, 1H) 2.44-2.57
(m, 1H) 2.23-2.32 (m, 3H) 1.83-2.00 (m, 5H) 1.43-1.61 (m, 2H) 1.09-1.21 (m, 2H)
m/z = 468.5 (M + 1)

| | | | | | |
|---|---|---|---|---|---|
| 6K | H | H | — | 0 | 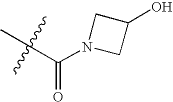 |

m/z = 452.5 (M + 1)

| | | | | | |
|---|---|---|---|---|---|
| 6L | H | H | — | 0 | 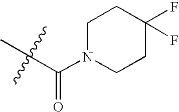 |

1H NMR (400 MHz, CHLOROFORM-d) δ ppm 8.35 (s, 1H) 7.93 (br. s., 1H) 7.32-7.16 (m, 4H) 5.65 (br. s., 1H)
4.62-4.68 (m, 2H) 3.95-4.00 (m, 2H) 3.66-3.74 (m, 2H) 3.56-3.60 (m, 2H) 3.31-3.35 (m, 1H) 3.07-3.13 (m, 1H)
2.12-2.51 (m, 4H), 1.80-2.03 (m, 8H), 1.42-1.58 (m, 1H), 1.05-1.14 (m, 1H),
m/z = 500.5 (M + 1)

| | | | | | |
|---|---|---|---|---|---|
| 6M | H | H | — | 0 | 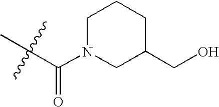 |

m/z = 494 (M + 1)
HPLC Retention Time = 2.705 (ymc ODS-AW 2.0 33 50 mm 5μm/0.05% TFA)

| | | | | | |
|---|---|---|---|---|---|
| 6N | H | H | — | 0 | 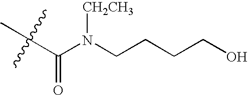 |

m/z = 496 (M + 1)
HPLC Retention Time = 2.767 (ymc ODS-AW 2.0 × 50 mm 5μm/0.05% TFA)

| | | | | | |
|---|---|---|---|---|---|
| 6O | H | H | — | 0 | 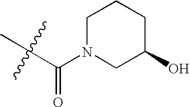 |

m/z = 480 (M + 1):
HPL Retention Time = 2.627 (ymc ODS-AW 2.0 × 50 mm 5μm/0.05% TFA)

| | | | | | |
|---|---|---|---|---|---|
| 6P | H | H | — | 0 | 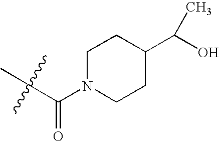 |

m/z = 508 (M + 1)
HPLC Retention Time = 2.725 (ymc ODS-AW 2.0 × 50 mm 5μm/0.05% TFA)

| | | | | | |
|---|---|---|---|---|---|
| 6Q | H | H | — | 0 | 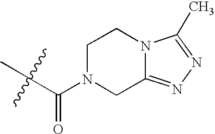 |

m/z = 517 (M + 1)
HPLC Retention Time = 2.411 (ymc ODS-AW 2.0 × 50 mm 5μm/0.05% TFA)

| | | | | | |
|---|---|---|---|---|---|
| 6R | H | H | — | 0 | 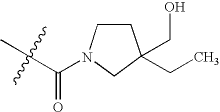 |

m/z = 508 (M + 1)
HPLC Retention Time = 2.802 (ymc ODS-AW 2.0 × 50 mm 5μm/0.05% TFA)

| | | | | | |
|---|---|---|---|---|---|
| 6S | H | H | — | 0 | 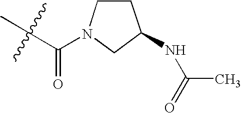 |

m/z = 570 (M + 1)
HPLC Retention time = 2.552 (ymc ODS-AW 2.0 × 50 mm 5μm/0.05% TFA)

| | | | | | |
|---|---|---|---|---|---|
| 6T | H | H | — | 0 | 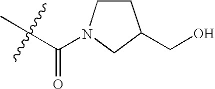 |

m/z = 480 (M + 1)
HPLC Retention Time = 2.596 (ymc ODS-AW 2.0 × 50 mm 5μm/0.05% TFA)

| | | | | | |
|---|---|---|---|---|---|
| 6U | H | H | — | 0 | 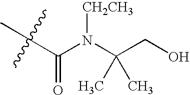 |

m/z = 496 (M + 1)
HPLC Retention Time = 2.193 (ymc ODS-AW 2.0 × 50 mm 5μm/0.05% TFA)

| | | | | | |
|---|---|---|---|---|---|
| 6V | H | H | — | 0 | 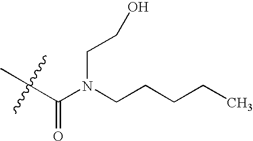 |

m/z = 510 (M + 1)
HPLC Retention Time = 2.553 (ymc ODS-AW 2.0 × 50 mm 5μm/0.05% TFA)

| | | | | | |
|---|---|---|---|---|---|
| 6W | H | H | — | 0 | 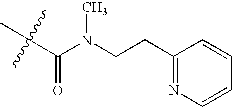 |

Isolated as the trifluoroacetate salt
m/z = 515 (M + 1)
HPLC Retention Time = 2.093 (ymc ODS-AW 2.0 × 50 mm 5μm/0.05% TFA)

| | | | | | |
|---|---|---|---|---|---|
| 6X | H | H | — | 0 | 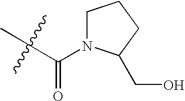 |

m/z = 480 (M + 1)
HPLC Retention Time = 2.389 (ymc ODS-AW 2.0 × 50 mm 5μm/0.05% TFA)

| | | | | | |
|---|---|---|---|---|---|
| 6Y | H | H | — | 0 | 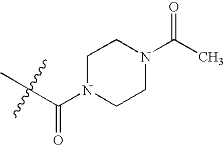 |

m/z = 507 (M + 1)
HPLC Retention Time = 2.241 (ymc ODS-AW 2.0 × 50 mm 5μm/0.05% TFA)

| | | | | | |
|---|---|---|---|---|---|
| 6Z | H | H | — | 0 | 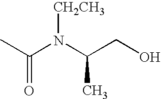 |

m/z = 482 (M + 1)
HPLC Retention Time = 2.884 (Welch XB-C18 2.1 × 50 mm 5μm/0.5% NH₄OH)

| | | | | | |
|---|---|---|---|---|---|
| 6AA | H | H | — | 0 | 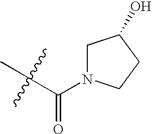 |

m/z = 466 (M + 1)
HPLC Retention Time = 2.2 (ymc ODS-AW 2.0 × 50 mm 5μm/0.05% TFA)

| | | | | | |
|---|---|---|---|---|---|
| 6AB | H | H | — | 0 | 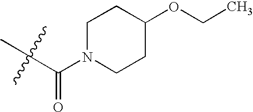 |

m/z = 508 (M + 1)
HPLC Retention Time = 2.654 (ymc ODS-AW 2.0 × 50 mm 5μm/0.05% TFA)

| | | | | | |
|---|---|---|---|---|---|
| 6AC | H | H | — | 0 | 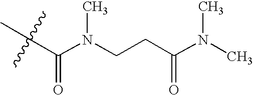 |

m/z = 509 (M + 1)
HPLC Retention Time = 2.314 (ymc ODS-AW 2.0 × 50 mm 5μm/0.05% TFA)

| | | | | | |
|---|---|---|---|---|---|
| 6AD | H | H | — | 0 | 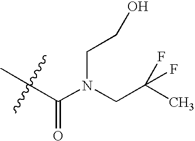 |

m/z = 518 (M + 1)
HPLC Retention Time = 2.475 (ymc ODS-AW 2.0 × 50 mm 5μm/0.05% TFA)

| | | | | | |
|---|---|---|---|---|---|
| 6AE | H | H | — | 0 | 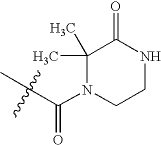 |

m/z = 507 (M + 1)
HPLC Retention Time = 2.31 (ymc ODS-AW 2.0 × 50 mm 5μm/0.05% TFA)

| | | | | | |
|---|---|---|---|---|---|
| 6AF | H | H | — | 0 | 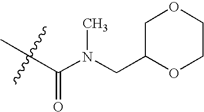 |

m/z = 510 (M + 1)
HPLC Retention Time = 2.456 (ymc ODS-AW 2.0 × 50 mm 5μm/0.05% TFA)

| | | | | | |
|---|---|---|---|---|---|
| 6AG | H | H | — | 0 | 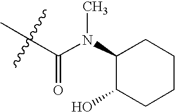 |

m/z = 508 (M + 1)
HPLC Retention Time = 2.537 (ymc ODS-AW 2.0 × 50 mm 5μm/0.05% TFA)

| | | | | | |
|---|---|---|---|---|---|
| 6AH | H | H | — | 0 | 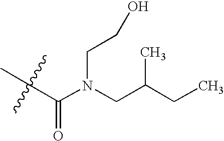 |

m/z = 510 (M + 1)
HPLC Retention Time = 2.515 (ymc ODS-AW 2.0 × 50 mm 5μm/0.05% TFA)

| | | | | | |
|---|---|---|---|---|---|
| 6AI | H | H | — | 0 | 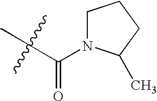 |

m/z = 464 (M + 1)
HPLC Retention Time = 2.685 (ymc ODS-AW 2.0 × 50 mm 5μm/0.05% TFA)

| | | | | | |
|---|---|---|---|---|---|
| 6AJ | H | H | — | 0 | 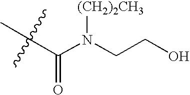 |

m/z = 482 (M + 1)
HPLC Retention Time = 2.468 (ymc ODS-AW 2.0 × 50 mm 5μm/0.05% TFA)

| | | | | | |
|---|---|---|---|---|---|
| 6AK | H | H | — | 0 | 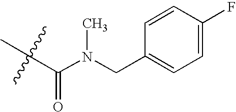 |

m/z = 518 (M + 1)
HPLC Retention Time = 2.708 (ymc ODS-AW 2.0 × 50 mm 5μm/0.05% TFA)

| | | | | | |
|---|---|---|---|---|---|
| 6AL | H | H | — | 0 | 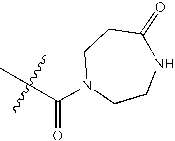 |

m/z = 493 (M + 1)
HPLC Retention Time = 2.141 (ymc ODS-AW 2.0 × 50 mm 5μm/0.05% TFA)

| | | | | | |
|---|---|---|---|---|---|
| 6AM | H | H | — | 0 | 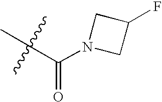 |

m/z = 454 (M + 1)
HPLC Retention Time = 2.398 (ymc ODS-AW 2.0 × 50 mm 5μm/0.05% TFA)

| | | | | | |
|---|---|---|---|---|---|
| 6AN | H | H | — | 0 | 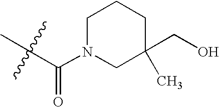 |

m/z = 508 (M + 1)
HPLC Retention Time = 2.496 (ymc ODS-AW 2.0 × 50 mm 5μm/0.05% TFA)

| | | | | | |
|---|---|---|---|---|---|
| 6AO | H | H | — | 0 | 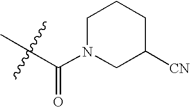 |

m/z = 489 (M + 1)
HPLC Retention Time = 2.465 (ymc ODS-AW 2.0 × 50 mm 5μm/0.05% TFA)

| | | | | | |
|---|---|---|---|---|---|
| 6AP | H | H | — | 0 | 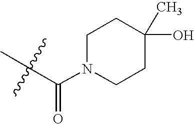 |

m/z = 494 (M + 1)
HPLC Retention Time = 2.333 (ymc ODS-AW 2.0 × 50 mm 5μm/0.05% TFA)

| | | | | | |
|---|---|---|---|---|---|
| 6AQ | H | H | — | 0 | 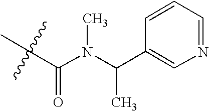 |

Isolated as the trifluoroacetate salt
m/z = 515 (M + 1)
HPLC Retention Time = 2.153 (ymc ODS-AW 2.0 × 50 mm 5μm/0.05% TFA)

| | | | | | |
|---|---|---|---|---|---|
| 6AR | H | H | — | 0 | 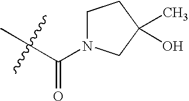 |

m/z = 480 (M + 1)
HPLC Retention Time = 2.288 (ymc ODS-AW 2.0 × 50 mm 5μm/0.05% TFA)

| | | | | | |
|---|---|---|---|---|---|
| 6AS | H | H | — | 0 | 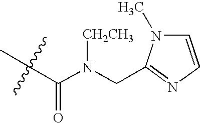 |

Isolated as the trifluoroacetate salt
m/z = 518 (M + 1)
HPLC Retention Time = 2.116 (ymc ODS-AW 2.0 × 50 mm 5μm/0.05% TFA)

| | | | | | |
|---|---|---|---|---|---|
| 6AT | H | H | — | 0 | 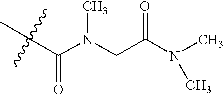 |

m/z = 495 (M + 1)
HPLC Retention Time = 2.278 (ymc ODS-AW 2.0 × 50 mm 5μm/0.05% TFA)

| | | | | | |
|---|---|---|---|---|---|
| 6AU | H | H | — | 0 | 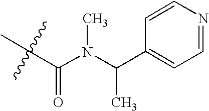 |

Isolated as the trifluoroacetate salt
m/z = 515 (M + 1)
HPLC Retention Time = 2.138 (ymc ODS-AW 2.0 × 50 mm 5μm/0.05% TFA)

| | | | | | |
|---|---|---|---|---|---|
| 6AV | H | H | — | 0 | 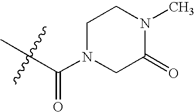 |

m/z = 493 (M + 1)
HPLC Retention Time = 2.21 (ymc ODS-AW 2.0 × 50 mm 5μm/0.05% TFA)

| | | | | | |
|---|---|---|---|---|---|
| 6AW | H | H | — | 0 | 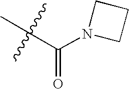 |

m/z = 436 (M + 1)
HPLC Retention Time = 2.408 (ymc ODS-AW 2.0 × 50 mm 5μm/0.05% TFA)

| | | | | | |
|---|---|---|---|---|---|
| 6AX | H | H | — | 0 | 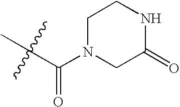 |

m/z = 479 (M + 1)
HPLC Retention Time = 2.122 (ymc ODS-AW 2.0 × 50 mm 5μm/0.05% TFA)

| | | | | | |
|---|---|---|---|---|---|
| 6AY | H | H | — | 0 | 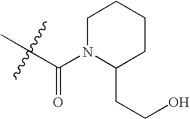 |

m/z = 508 (M + 1)
HPLC Retention Time = 2.457 (Welch XB-C18 2.1 × 50 mm 5μm/0.5% NH₄OH)

| | | | | | |
|---|---|---|---|---|---|
| 6AZ | H | H | — | 0 | 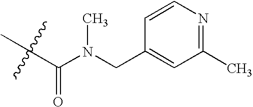 |

Isolated as the trifluoroacetate salt
m/z = 515 (M + 1)
HPLC Retention Time = 2.096 (ymc ODS-AW 2.0 × 50 mm 5μm/0.05% TFA)

| | | | | | |
|---|---|---|---|---|---|
| 6BA | H | H | — | 0 | 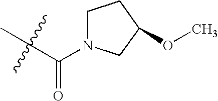 |

m/z = 480 (M + 1)
HPLC Retention Time = 2.531 (ymc ODS-AW 2.0 × 50 mm 5μm/0.05% TFA)

| | | | | | |
|---|---|---|---|---|---|
| 6BB | H | H | — | 0 | 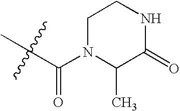 |

m/z = 493 (M + 1)
HPLC Retention Time = 2.531 (ymc ODS-AW 2.0 × 50 mm 5μm/0.05% TFA)

| | | | | | |
|---|---|---|---|---|---|
| 6BC | H | H | — | 0 | 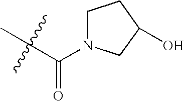 |

m/z = 466 (M + 1)
HPLC Retention Time = 2.531 (ymc ODS-AW 2.0 × 50 mm 5μm/0.05% TFA)

| | | | | | |
|---|---|---|---|---|---|
| 6BD | H | H | — | 0 | 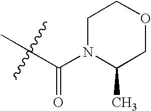 |

m/z = 480 (M + 1)
HPLC Retention Time = 2.771 (ymc ODS-AW 2.0 × 50 mm 5μm/0.05% TFA)

| | | | | | |
|---|---|---|---|---|---|
| 6BE | H | H | — | 0 | 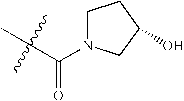 |

m/z = 466 (M + 1)
HPLC Retention Time = 2.532 (ymc ODS-AW 2.0 × 50 mm 5μm/0.05% TFA)

| | | | | | |
|---|---|---|---|---|---|
| 6BF | H | H | — | 0 | 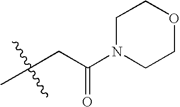 |

1H NMR (400 MHz, CHLOROFORM-d) δ ppm 0.98-1.15 (m, 2H) 1.25- 1.37 (m, 1H) 1.36-1.50
(m, 2H) 1.50-1.61 (m, 2H) 1.79-1.92 (m, 4H) 2.24- 2.38 (m, 2H) 2.40-2.56 (m, 1H) 3.37-3.51
(m, 2H) 3.52-3.68 (m, 6H) 3.90-4.01 (m, 2H) 4.65 (dt, J = 4.98, 3.81 Hz, 2H) 5.63 (br. s., 1H)
7.10-7.32 (m, 4H) 8.13 (s, 1H) 8.24 (s, 1H).
m/z = 480.5 (M + 1)

| | | | | | |
|---|---|---|---|---|---|
| 6BG | H | CH₃ | — | 0 | —C(O)N(CH₃)₂ |

1H NMR (400 MHz, CHLOROFORM-d) δ ppm 1.44 (d, J = 6.64 Hz, 3H) 3.02 (d, 6H) 3.75-3.98
(m, 2H) 4.81-5.01 (m, 1H) 5.59 (br. s., 1H) 7.33 (d, J = 8.20 Hz, 2H) 7.51 (d, J = 8.39 Hz, 2H) 7.93
(br. s., 1H) 8.30 (s, 1H).
m/z = 342.3 (M + 1)

### Example 7

### Preparation of 6-[4-(1-acetylpiperidin-4-yl)phenyl]-4-amino-7,8-dihydropyrimido[5,4-f][1,4]oxazepin-5(6H)-one (7A)

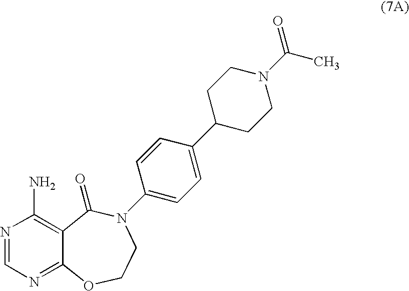

A solution of acetic acid (6 mg, 0.01 mmole), 4-[4-(4-amino-5-oxo-7,8-dihydropyrimido[5,4-f][1,4]oxazepin-6(5H)-yl)phenyl]piperidine (I-3b: 17 mg, 0.05 mmole), 2-(1H-7-azabenzotriazol-1-yl)-1,1,3,3-tetramethyl uronium hexafluorophosphate methanaminium, also known as HATU, (38 mg, 0.1 mmole) and triethylamine (20 mg, 0.2 mmole) in DMF (0.5 mL) was stirred for 18 hours. The reaction mixtures were concentrated in vacuo and purified by reverse phase HPLC to afford the title compound (7A), 12 mg. m/z=382.1 (M+1).

The compounds listed in Table 4 below were prepared using procedures analogous to those described above for the synthesis of 6-[4-(1-acetylpiperidin-4-yl)phenyl]-4-amino-7,8-dihydropyrimido[5,4-f][1,4]oxazepin-5(6H)-one ON using the appropriate starting materials which are available commercially, prepared using preparations well-known to those skilled in the art, or prepared in a manner analogous to routes described above for other intermediates. The structures were verified by high resolution mass spectrometry.

TABLE 4

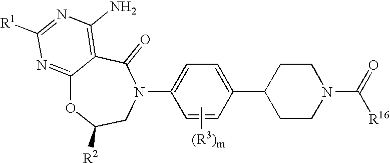

| Ex. No. | R¹ | R² | R³ | m | R¹⁶ |
|---|---|---|---|---|---|
| 7B | H | H | — | 0 | —OC(CH₃)₃ |

1H NMR (400 MHz, CHLOROFORM-d) δ ppm 1.44 (s, 9H)
1.50-1.65 (m, 2H) 1.70-1.85 (m, 2H) 2.57-2.70 (m, 1H)
2.70-2.86 (m, 2H) 3.91-4.04 (m, 2H) 4.13-4.31 (m, 2H)
4.59-4.75 (m, 2H) 5.60 (br. s., 1H) 7.19 (d, 2H)
7.27 (d, 2H) 8.08 (br. s., 1H) 8.24 (s, 1H)
m/z = 440.3 (M + 1)

| | | | | | |
|---|---|---|---|---|---|
| 7C | H | H | — | 0 | 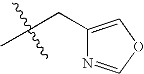 |

m/z = 465.1 (M + 1)

| | | | | | |
|---|---|---|---|---|---|
| 7D | H | H | — | 0 | 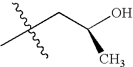 |

m/z = 426.1 (M + 1)

| | | | | | |
|---|---|---|---|---|---|
| 7E | H | H | — | 0 | 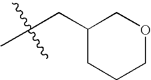 |

m/z = 466.2 (M + 1)

| | | | | | |
|---|---|---|---|---|---|
| 7F | H | H | — | 0 | 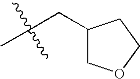 |

m/z = 438.1 (M + 1)

| | | | | | |
|---|---|---|---|---|---|
| 7G | H | H | — | 0 | 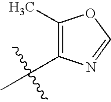 |

m/z = 449.1 (M + 1)

| | | | | | |
|---|---|---|---|---|---|
| 7H | H | H | — | 0 | 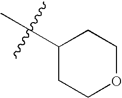 |

m/z = 452.1 (M + 1)

| | | | | | |
|---|---|---|---|---|---|
| 7I | H | H | — | 0 | 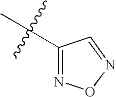 |

m/z = 436.1 (M + 1)

| | | | | | |
|---|---|---|---|---|---|
| 7J | H | H | — | 0 | 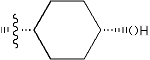 |

m/z = 466.2 (M + 1)

| | | | | | |
|---|---|---|---|---|---|
| 7K | H | H | — | 0 | 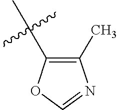 |

m/z = 449.1 (M + 1)

| | | | | | |
|---|---|---|---|---|---|
| 7L | H | H | — | 0 | 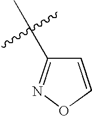 |

m/z = 435.1 (M + 1)

| | | | | | |
|---|---|---|---|---|---|
| 7M | H | H | — | 0 | 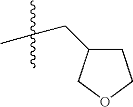 |

m/z = 452.1 (M + 1)

| | | | | | |
|---|---|---|---|---|---|
| 7N | H | H | — | 0 | 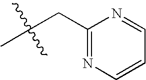 |

m/z = 460.1 (M + 1)

| | | | | | |
|---|---|---|---|---|---|
| 7O | H | H | — | 0 | 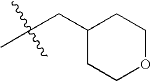 |

m/z = 466.2 (M + 1)

| | | | | | |
|---|---|---|---|---|---|
| 7P | H | H | — | 0 | 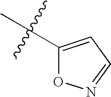 |

m/z = 435.1 (M + 1)

| | | | | | |
|---|---|---|---|---|---|
| 7Q | H | H | — | 0 | —CH₂OCH₂CH₃ |

m/z = 426.1 (M + 1)

| | | | | | |
|---|---|---|---|---|---|
| 7R | H | H | — | 0 | 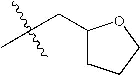 |

m/z = 452.1 (M + 1)

| | | | | | |
|---|---|---|---|---|---|
| 7S | H | H | — | 0 | —CH(CH₃)₂ |

m/z = 410.1 (M + 1)

| | | | | | |
|---|---|---|---|---|---|
| 7T | H | H | — | 0 | —CH₂SO₂CH₃ |

m/z = 460.1 (M + 1)

| | | | | | |
|---|---|---|---|---|---|
| 7U | H | H | — | 0 | 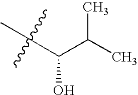 |

m/z = 440.1 (M + 1)

| | | | | | |
|---|---|---|---|---|---|
| 7V | H | H | — | 0 | 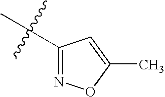 |

m/z = 449.1 (M + 1)

| | | | | | |
|---|---|---|---|---|---|
| 7W | H | H | — | 0 | 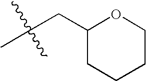 |

m/z = not available

| | | | | | |
|---|---|---|---|---|---|
| 7X | H | H | — | 0 | 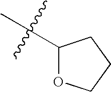 |

m/z = not available

| | | | | | |
|---|---|---|---|---|---|
| 7Y | H | H | — | 0 | benzyl |

m/z = 458.1 (M + 1)

| | | | | | |
|---|---|---|---|---|---|
| 7Z | H | H | — | 0 | 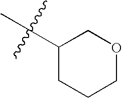 |

m/z = 452.1 (M + 1)

| | | | | | |
|---|---|---|---|---|---|
| 7AA | H | H | — | 0 | cyclohexyl |

m/z = 450.2 (M + 1)

| | | | | | |
|---|---|---|---|---|---|
| 7AB | H | H | — | 0 | 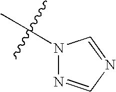 |

m/z = 449.1 (M + 1)

| | | | | | |
|---|---|---|---|---|---|
| 7AC | H | H | — | 0 | 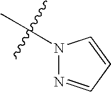 |

m/z = not available

| | | | | | |
|---|---|---|---|---|---|
| 7AD | H | H | — | 0 | phenyl |

m/z = not available

| | | | | | |
|---|---|---|---|---|---|
| 7AE | H | H | — | 0 | 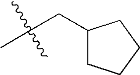 |

m/z = not available

### Pharmacological Testing

The practice of the instant invention for the treatment of diseases modulated by the inhibition of DGAT-1 can be evidenced by activity in at least one of the protocols described hereinbelow.

### In Vitro Assay for Inhibition of DGAT-1 Activity

Human full-length diacylglycerol:acylCoA acyltransferase 1 (DGAT-1) was expressed in Sf9 insect cells which are then lysed and a crude membrane fraction (105,000×g pellet) was prepared. The DGAT-1 gene is a human DGAT-1 gene described in J Biol Chem 273:26765 (1998) and U.S. Pat. No. 6,100,077.

In vitro inhibition of DGAT-1 was measured using a modification, further described below, of the assay methodology described in U.S. Pat. No. 6,994,956 B2.

The cells were cultured as follows. Sf9 cells (20 L) were infected with 4 mL of DGAT1 Baculovirus Infected Insect Cells (BIIC) for 72 hours in a Wave Bioreactor System 20/50P (Wave Biotec/GE Healthcare™).

Crude DGAT-1 microsomes were prepared as follows. Cell pellets were washed once with ice-cold Dulbecco's phosphate-buffered saline. Cells were collected in tabletop centrifuge (Beckmann' GS-6KR), 15 minutes, 2000×g, 4° C. Twenty (20) mL of ice-cold Microsome Buffer (MB) was added per 5 g of cell pellet. The suspension was passed through a microfluidizer 3 times (18K psi). The lysate was transferred to centrifuge tubes and centrifuged for 20 minutes at 5000×g (Beckman-Coulter, Inc. Allegra® 64R High-Speed Refrigerated Benchtop Centrifuge, F0650 rotor) at 4° C. The supernatant was transferred to ultracentrifuge tubes and centrifuged at 125,000×g for 1 hour in a Beckman™ Ti-45 rotor, 4° C. The supernatant fluid was discarded. The pellet was resuspended in 70 mL of MB by sonication. The microsome concentration was determined using Bio-Rad Protein DC Protein Assay. The samples were portioned, flash frozen and stored at −80° C.

The Microsome Buffer, used for microsome preparation, was prepared by conventional means and contained 125 mM sucrose, 3 mM imidazole, 0.2 μg/mL aprotinin, 0.2 μg/mL leupeptin and 5 mM dithiothreitol (Cleland's reagent) at pH −7.4 DGAT-1 activity was measured in 384-well format in a total assay volume of 20 μl that contained, Hepes buffer (50 mM, pH 7.5), MgCl₂ (10 mM), bovine serum albumin (0.6 mg/ml), [¹⁴C]decanoylCoA (25 μM, 58 Ci/mol) and microsomes (5.6 μg/ml) into which 1,2 dioleoyl-sn-glycerol (75 μM) in acetone has already been incorporated. Inhibitors in DMSO were pre-incubated with membranes before initiating the DGAT-1 reaction by the addition of decanoylCoA. Two control DGAT-1 reactions were also incubated in parallel: 1) DMSO without inhibitor to measure zero percent effect of inhibition and 2) and a maximally inhibited DGAT-1 reaction (“blank”) incubated with 1 μM {trans-4-[4-(4-amino-2,7,7-trimethyl-7H-pyrimido[4,5-b][1,4]oxazin-6-yl)phenyl]cyclohexyl}acetic acid (WO2004/047755), which was the 100 percent effect sample. The concentration of dimethylsulfoxide (DMSO) in the reaction mix was 2.5%. The inhibitors were present at a range of eight concentrations to generate an apparent IC₅₀ for each compound. The eight inhibitor concentration employed ranged from 3 μM to 1 nM (from high to low concentration). Specifically, the eight concentrations used were 3 μM, 1 μM, 300 nM, 100 nM, 30 nM, 10 nM, 3 nM and 1 nM.

The reactions were allowed to proceed for 1.5 hours at room temperature and then terminated by the addition of 20 μl of EDTA (40 mM). Reaction mixture is then mixed by trituration with 30 μl of Microscint™-E (Perkin Elmer). Plates contents were allowed to partition for 15 to 30 minutes before ¹⁴C was measured in a scintillation spectrometer (Wallac Microbeta Trilux 1450-030, 12 detector in the top-count DPM mode). Percent inhibition of test compounds was computed as 100−((DPM DMSO uninhibited−DPM test compound)/(DPM DMSO uninhibited)). Four separate trials were conducted. The method of analysis of Trial 1 was the same as Trial 4 (described above) except microsomes were utilized at 25 μg/mL instead of 5 μg/mL. The method of analysis of Trial 2 was the same as Trial 4 (described above) except eleven (11) concentrations of inhibitor were employed instead of eight (8). The method of analysis of Trial 3 was the same as Trial 2 except the compounds were serially diluted in a different laboratory.

The compounds of the present invention, described in Examples above (except Example 7W) were tested for in vitro DGAT-1 inhibition, and were found to exhibit DGAT-1 inhibition with IC₅₀ values provided below in Table 5. Where this DGAT-1 inhibition assay was performed on a compound more than once, an average is provided for that compound. Preferably, the compounds of the present invention exhibit DGAT-1 inhibition with IC₅₀ values of 100 nM or less.

TABLE 5
DGAT 1 Reduced Microsome Multidose Assay Results

| | | Trial 1 | Trial 2 | Trial 3 | Trial 4 |
|---|---|---|---|---|---|
| Ex. | | Human | Human | Human | Human |
| No. | Structural Name | IC50 | IC50 | IC50 | IC50 |
| 1A | 4-amino-6-{4-[trans-4-(2-hydroxy- | 24.9 nm | — | 92.0 nm | — |
| | 2-methylpropyl)cyclohexyl]- | (n = 4) | | (n = 2) | |
| | phenyl}-7,8-dihydropyrimido[5,4- | | | | |
| | f][1,4]oxazepin-5(6H)-one | | | | |
| 1B | 4-amino-6-{4-[trans-4-(2-amino-2- | — | 19.8 nm | 39.9 nm | — |
| | methylpropyl)-cyclohexyl]phenyl}- | | (n = 2) | (n = 3) | |
| | 7,8-dihydropyrimido[5,4-f]- | | | | |
| | [1,4]oxazepin-5(6H)-one | | | | |
| 1C | 4-amino-6-(4-tert-butylphenyl)- | 134 nm | 14.5 nm | 39.7 nm | — |
| | 7,8-dihydropyrimido[5,4-f]- | (n = 2) | (n = 2) | (n = 1) | |
| | [1,4]oxazepin-5(6H)-one | | | | |
| 1D | 2-[4-(4-amino-5-oxo-7,8- | 2750 nm | — | — | — |
| | dihydropyrimido[5,4-f]- | (n = 1) | | | |
| | [1,4]oxazepin-6(5H)-yl)phenyl]-2- | | | | |
| | methylpropanamide | | | | |
| 1E | 4-amino-6-{4-[trans-4-(2- | — | 14.1 nm | 9.75 nm | — |
| | hydroxyethyl)cyclohexyl]-phenyl}- | | (n = 1) | (n = 2) | |
| | 7,8-dihydro-pyrimido[5,4-f]- | | | | |
| | [1,4]oxazepin-5(6H)-one | | | | |
| 1F | 2-[4-(4-amino-5-oxo-7,8- | 2120 nm | 1290 nm | 2660 nm | — |
| | dihydropyrimido[5,4-f]- | (n = 1) | (n = 1) | (n = 2) | |
| | [1,4]oxazepin-6(5H)-yl)-phenyl]-2- | | | | |
| | methyl-propanenitrile | | | | |
| 1G | 4-amino-6-(4-methylphenyl)-7,8- | 2210 nm | 265 nm | 244 nm | — |
| | dihydropyrimido[5,4-f]- | (n = 1) | (n = 1) | (n = 2) | |
| | [1,4]oxazepin-5(6H)-one | | | | |
| 1H | 4-amino-6-(4-isopropylphenyl)- | — | 17.1 nm | 26.3 | — |
| | 7,8-dihydropyrimido[5,4-f]- | | (n = 1) | (n = 2) | |
| | [1,4]oxazepin-5(6H)-one | | | | |
| 1I | (8R)-4-amino-6-(4-tert- | — | 18.4 nm | 33.7 nm | 16.3 nm |
| | butylphenyl)-8-methyl-7,8- | | (n = 1) | (n = 5) | (n = 8) |
| | dihydropyrimido[5,4-f]- | | | | |
| | [1,4]oxazepin-5(6H)-one | | | | |
| 1J | methyl 4-[(8R)-4-amino-8-methyl- | — | — | — | 161 nm |
| | 5-oxo-7,8-dihydropyrimido[5,4- | | | | (n = 3) |
| | f][1,4]oxazepin-6(5H)-yl]benzoate | | | | |
| 1K | (8R)-4-amino-6-(4- | — | — | — | 17.6 nm |
| | isopropylphenyl)-8-methyl-7,8- | | | | (n = 3) |
| | dihydropyrimido[5,4-f]- | | | | |
| | [1,4]oxazepin-5(6H)-one | | | | |
| 1L | (8R)-4-amino-6-[4-(1-fluoro-1- | — | — | — | 46.1 nm |
| | methylethyl)phenyl]-8-methyl-7,8- | | | | (n = 2) |
| | dihydropyrimido[5,4- | | | | |
| | f][1,4]oxazepin-5(6H)-one | | | | |
| 1M | (8R)-4-amino-6-(4-ethylphenyl)-8- | — | — | — | 13.7 nm |
| | methyl-7,8-dihydropyrimido[5,4-f]- | | | | (n = 3) |
| | [1,4]oxazepin-5(6H)-one | | | | |
| 1N | (8R)-4-amino-6-(4-tert- | — | — | — | 44.5 nm |
| | butylphenyl)-2-methoxy-8-methyl- | | | | (n = 2) |
| | 7,8-dihydro-pyrimido[5,4-f]- | | | | |
| | [1,4]oxazepin-5(6H)-one | | | | |
| 1O | (8R)-4-amino-6-[4-(1- | — | — | — | 48.2 nm |
| | methoxycyclobutyl)phenyl]-8- | | | | (n = 3) |
| | methyl-7,8-dihydropyrimido[5,4-f]- | | | | |
| | [1,4]oxazepin-5(6H)-one | | | | |
| 1P | (8R)-4-amino-8-methyl-6-(4- | — | — | — | 52.8 nm |
| | methylphenyl)-7,8- | | | | (n = 2) |
| | dihydropyrimido[5,4-f]- | | | | |
| | [1,4]oxazepin-5(6H)-one | | | | |
| 1Q | (8R)-4-amino-6-[4-(cis-3- | — | — | — | 76.5 nm |
| | hydroxycyclobutyl)phenyl]-8- | | | | (n = 2) |
| | methyl-7,8-dihydropyrimido[5,4-f]- | | | | |
| | [1,4]oxazepin-5(6H)-one | | | | |
| 1R | (8R)-4-amino-8-methyl-6-[4- | — | — | — | 37.7 nm |
| | (2,2,2-trifluoro-1- | | | | (n = 2) |
| | hydroxyethyl)phenyl]-7,8- | | | | |
| | dihydropyrimido[5,4- | | | | |
| | f][1,4]oxazepin-5(6H)-one | | | | |
| 1S | (8R)-4-amino-6-(2,3-dihydro-1H- | — | — | — | <10.7 nm |
| | inden-5-yl)-8-methyl-7,8- | | | | (n = 6) |
| | dihydropyrimido[5,4-f]- | | | | |
| | [1,4]oxazepin-5(6H)-one | | | | |
| 1T | (8R)-4-amino-6-[4-(3,3- | — | — | — | <15.3 nm |
| | difluorocyclobutyl)phenyl]-8- | | | | (n = 6) |
| | methyl-7,8-dihydropyrimido[5,4-f]- | | | | |
| | [1,4]oxazepin-5(6H)-one | | | | |
| 1U | (8R)-4-amino-6-(4- | — | — | — | 19.9 nm |
| | cyclopropylphenyl)-8-methyl-7,8- | | | | (n = 4) |
| | dihydropyrimido[5,4-f]- | | | | |
| | [1,4]oxazepin-5(6H)-one | | | | |
| 1V | (8R)-4-amino-8-methyl-6-{4- | — | — | — | 39.5 nm |
| | [2,2,2-trifluoro-1-hydroxy-1- | | | | (n = 2) |
| | (trifluoromethyl)-ethyl]phenyl}-7,8- | | | | |
| | dihydropyrimido[5,4-f]- | | | | |
| | [1,4]oxazepin-5(6H)-one | | | | |
| 1W | (8R)-4-amino-6-[4-(1- | — | — | — | 146 nm |
| | hydroxyethyl)-phenyl]-8-methyl- | | | | (n = 2) |
| | 7,8-dihydro-pyrimido[5,4- | | | | |
| | f][1,4]oxazepin-5(6H)-one | | | | |
| 1X | methyl 2-{4-[(8R)-4-amino-8- | — | — | — | 32.5 nm |
| | methyl-5-oxo-7,8- | | | | (n = 2) |
| | dihydropyrimido[5,4-f]- | | | | |
| | [1,4]oxazepin-6(5H)-yl]phenyl}-2- | | | | |
| | methylpropanoate | | | | |
| 1Y | (8R)-4-amino-6-(4- | — | — | — | 6.78 nm |
| | isobutylphenyl)-8-methyl-7,8- | | | | (n = 4) |
| | dihydropyrimido[5,4-f]- | | | | |
| | [1,4]oxazepin-5(6H)-one | | | | |
| 1Z | (8R)-4-amino-8-methyl-6-[4- | — | — | — | 14.3 nM |
| | (2,2,2-trifluoro-1,1- | | | | (n = 5) |
| | dimethylethyl)phenyl]-7,8- | | | | |
| | dihydropyrimido[5,4- | | | | |
| | f][1,4]oxazepin-5(6H)-one | | | | |
| 1AA | (8R)-4-amino-6-[4-(2-methoxy- | — | — | — | 42.7 nM |
| | 1,1-dimethylethyl)phenyl]-8- | | | | (n = 4) |
| | methyl-7,8-dihydropyrimido[5,4- | | | | |
| | f][1,4]oxazepin-5(6H)-one | | | | |
| 1AB | (8R)-4-amino-6-(3,4- | — | — | — | 24.8 nM |
| | dichlorophenyl)-8-methyl-7,8- | | | | (n = 8) |
| | dihydropyrimido[5,4- | | | | |
| | F][1,4]oxazepin-5(6H)-one | | | | |
| 1AC | (8R)-4-amino-6-{4-[(1S)-1- | — | — | — | 55.4 nM |
| | hydroxy-2,2- | | | | (n = 8) |
| | dimethylpropyl]phenyl}-8-methyl- | | | | |
| | 7,8-dihydropyrimido[5,4- | | | | |
| | f][1,4]oxazepin-5(6H)-one | | | | |
| 1AD | (8R)-4-amino-6-{4-[(1R)-1- | — | — | — | 22.6 nM |
| | hydroxy-2,2- | | | | (n = 8) |
| | dimethylpropyl]phenyl}-8-methyl- | | | | |
| | 7,8-dihydropyrimido[5,4- | | | | |
| | f][1,4]oxazepin-5(6H)-one | | | | |
| 1AE | 1-{4-[(8R)-4-amino-8-methyl-5- | — | — | — | 82.4 nM |
| | oxo-7,8-dihydropyrimido[5,4- | | | | (n = 6) |
| | f][1,4]oxazepin-6(5H)- | | | | |
| | yl]phenyl}cyclohexanecarboxamide | | | | |
| 1AF | 1-{4-[(8R)-4-amino-8-methyl-5- | — | — | — | 117 nM |
| | oxo-7,8-dihydropyrimido[5,4- | | | | (n = 6) |
| | f][1,4]oxazepin-6(5H)- | | | | |
| | yl]phenyl}cyclopentanecarboxamide | | | | |
| 1AG | (8R)-4-amino-6-[4-(2-methoxy-2- | — | — | — | 45.2 nM |
| | methylpropyl)phenyl]-8-methyl- | | | | (n = 6) |
| | 7,8-dihydropyrimido[5,4- | | | | |
| | f][1,4]oxazepin-5(6H)-one | | | | |
| 1AH | 1-{4-[(8R)-4-amino-8-methyl-5- | — | — | — | 78.2 nM |
| | oxo-7,8-dihydropyrimido[5,4- | | | | (n = 8) |
| | f][1,4]oxazepin-6(5H)- | | | | |
| | yl]phenyl}cyclobutanecarboxamide | | | | |
| 1AI | (8R)-4-amino-6-[4-(1-ethyl-1- | — | — | — | 15.5 nM |
| | methoxypropyl)phenyl]-8-methyl- | | | | (n = 6) |
| | 7,8-dihydropyrimido[5,4- | | | | |
| | f][1,4]oxazepin-5(6H)-one | | | | |
| 1AJ | (8R)-4-amino-6-[4-(1- | — | — | — | 35.6 nM |
| | hydroxycyclohexyl)phenyl]-8- | | | | (n = 6) |
| | methyl-7,8-dihydropyrimido[5,4- | | | | |
| | f][1,4]oxazepin-5(6H)-one | | | | |
| 1AK | (8R)-4-amino-6-[4-(1-ethoxy-1- | — | — | — | 42.3 nM |
| | methylethyl)phenyl]-8-methyl-7,8- | | | | (n = 6) |
| | dihydropyrimido[5,4- | | | | |
| | f][1,4]oxazepin-5(6H)-one | | | | |
| 1AL | (8R)-4-amino-6-[4-(1-methoxy-1- | — | — | — | 34.6 nM |
| | methylethyl)phenyl]-8-methyl-7,8- | | | | (n = 8) |
| | dihydropyrimido[5,4- | | | | |
| | f][1,4]oxazepin-5(6H)-one | | | | |
| 1AM | 2-{4-[(8R)-4-amino-8-methyl-5- | — | — | — | 313 nM |
| | oxo-7,8-dihydropyrimido[5,4- | | | | (n = 6) |
| | f][1,4]oxazepin-6(5H)- | | | | |
| | yl]phenyl}acetamide | | | | |
| 1AN | (8R)-4-amino-6-[4-(2-hydroxy-2- | — | — | — | 104 nM |
| | methylpropyl)phenyl]-8-methyl- | | | | (n = 6) |
| | 7,8-dihydropyrimido[5,4- | | | | |
| | f][1,4]oxazepin-5(6H)-one | | | | |
| 1AO | 2-{4-[(8R)-4-amino-8-methyl-5- | — | — | — | 20.0 nM |
| | oxo-7,8-dihydropyrimido[5,4- | | | | (n = 6) |
| | f][1,4]oxazepin-6(5H)-yl]phenyl}-2- | | | | |
| | methylpropanoic acid | | | | |
| 1AP | (8R)-4-amino-6-[4-(1-ethyl-1- | — | — | — | 29.4 nM |
| | hydroxypropyl)phenyl]-8-methyl- | | | | (n = 6) |
| | 7,8-dihydropyrimido[5,4- | | | | |
| | f][1,4]oxazepin-5(6H)-one | | | | |
| 1AQ | (8R)-4-amino-6-[4-(2-hydroxy-1,1- | — | — | — | 31.0 nM |
| | dimethylethyl)phenyl]-8-methyl- | | | | (n = 6) |
| | 7,8-dihydropyrimido[5,4- | | | | |
| | f][1,4]oxazepin-5(6H)-one | | | | |
| 1AR | 2-{4-[(8R)-4-amino-8-methyl-5- | — | — | — | 24.0 nM |
| | oxo-7,8-dihydropyrimido[5,4- | | | | (n = 6) |
| | f][1,4]oxazepin-6(5H)-yl]phenyl}-2- | | | | |
| | methylpropanamide | | | | |
| 1A-1 | 4-amino-6-{4-[trans-4-(3-hydroxy- | 104 nm | — | — | — |
| | 3-methylbutyl)cyclohexyl]phenyl}- | (n = 1) | | | |
| | 7,8-dihydropyrimido[5,4- | | | | |
| | f][1,4]oxazepin-5(6H)-one | | | | |
| 1A-2 | (8R)-4-amino-6-[4-(1-hydroxy-1- | — | — | — | 61.4 nm |
| | methylethyl)phenyl]-8-methyl-7,8- | | | | (n = 3) |
| | dihydropyrimido[5,4- | | | | |
| | f][1,4]oxazepin-5(6H)-one | | | | |
| 2A | 4-amino-6-(4-{trans-4-[(3-methyl- | 60 nm | 6.05 nm | 14.8 nm | 20.4 nm |
| | 1,2,4-oxadiazol-5-yl)methyl]- | (n = 7) | (n = 2) | (n = 5) | (n = 1) |
| | cyclohexyl}phenyl)-7,8-dihydro- | | | | |
| | pyrimido[5,4-f][1,4]oxazepin- | | | | |
| | 5(6H)-one | | | | |
| 2B | 4-amino-6-(4-{trans-4-[2-(3- | 36.8 nm | — | — | — |
| | methyl-1,2,4-oxadiazol-5-yl)ethyl]- | (n = 3) | | | |
| | cyclohexyl}-phenyl)-7,8- | | | | |
| | dihydropyrimido[5,4-f]- | | | | |
| | [1,4]oxazepin-5(6H)-one | | | | |
| 2C | (8R)-4-amino-8-methyl-6-(4- | — | — | — | 8.1 nm |
| | {trans-4-[(3-methyl-1,2,4- | | | | (n = 4) |
| | oxadiazol-5-yl- | | | | |
| | methyl]cyclohexyl}phenyl)-7,8- | | | | |
| | dihydropyrimido[5,4- | | | | |
| | f][1,4]oxazepin-5(6H)-one | | | | |
| 3A | 4-amino-6-(4-{trans-4-[(5-methyl- | 210 nm | — | — | — |
| | 1,3,4-oxadiazol-2-yl)methyl]- | (n = 3) | | | |
| | cyclohexyl}phenyl)-7,8-dihydro- | | | | |
| | pyrimido[5,4-f][1,4]oxazepin- | | | | |
| | 5(6H)-one | | | | |
| 4A | 4-Amino-6-(4-{trans-4-[(5-methyl- | 185 nm | — | — | — |
| | 1,3,4-thiadiazol-2-yl)methyl]- | (n = 3) | | | |
| | cyclohexyl}phenyl)-7,8-dihydro- | | | | |
| | pyrimido[5,4-f][1,4]oxazepin- | | | | |
| | 5(6H)-one | | | | |
| 5A | 4-amino-6-(4-{trans-4-[(4,5- | 312 nm | — | — | — |
| | dimethyl-4H-1,2,4-triazol-3- | (n = 3) | | | |
| | yl)methyl]cyclohexyl}phenyl)-7,8- | | | | |
| | dihydropyrimido[5,4- | | | | |
| | f][1,4]oxazepin-5(6H)-one | | | | |
| 5B | 4-amino-6-(4-{trans-4-[(5-methyl- | 391 nm | — | — | — |
| | 4H-1,2,4-triazol-3-yl)methyl]- | (n = 1) | | | |
| | cyclohexyl}-phenyl)-7,8-dihydro- | | | | |
| | pyrimido[5,4-f][1,4]oxazepin- | | | | |
| | 5(6H)-one | | | | |
| 6A | 4-amino-6-{4-[trans-4-(2-oxo-2- | 44.6 nm | — | — | — |
| | pyrrolidin-1-ylethyl)cyclohexyl]- | (n = 4) | | | |
| | phenyl}-7,8-dihydropyrimido[5,4- | | | | |
| | f]-[1,4]oxazepin-5(6H)-one | | | | |
| 6B | N-({trans-4-[4-(4-amino-5-oxo-7,8- | 25.7 nm | — | — | — |
| | dihydropyrimido[5,4- | (n = 3) | | | |
| | f][1,4]oxazepin-6(5H)- | | | | |
| | yl)phenyl]cyclohexyl}acetyl)-N- | | | | |
| | methylglycine | | | | |
| 6C | 4-amino-6-(4-{trans-4-[2-(4- | 49.6 nm | — | — | — |
| | methoxypiperidin-1-yl)-2- | (n = 3) | | | |
| | oxoethyl]-cyclohexyl}phenyl)-7,8- | | | | |
| | dihydro-pyrimido[5,4- | | | | |
| | f][1,4]oxazepin-5(6H)-one | | | | |
| 6D | 2-{trans-4-[4-(4-amino-5-oxo-7,8- | 103 nm | — | — | — |
| | dihydropyrimido[5,4- | (n = 3) | | | |
| | f][1,4]oxazepin-6(5H)- | | | | |
| | yl)phenyl]cyclohexyl}-N-methyl-N- | | | | |
| | (tetrahydro-2H-pyran-4- | | | | |
| | ylmethyl)acetamide | | | | |
| 6E | 4-amino-6-[4-(trans-4-{2-[(3S)-3- | 72.1 nm | — | — | — |
| | methoxypyrrolidin-1-yl]-2- | (n = 4) | | | |
| | oxoethyl}-cyclohexyl)phenyl]-7,8- | | | | |
| | dihydro-pyrimido[5,4- | | | | |
| | f][1,4]oxazepin-5(6H)-one | | | | |
| 6F | 4-amino-6-[4-(trans-4-{2-[(3S)-3- | 84.9 nm | — | — | — |
| | (methoxymethyl)pyrrolidin-1-yl]-2- | (n = 3) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6G | 1-({trans-4-[4-(4-amino-5-oxo-7,8- | 50.1 nm | — | — | — |
| | dihydropyrimido[5,4- | (n = 2) | | | |
| | f][1,4]oxazepin-6(5H)- | | | | |
| | yl)phenyl]cyclohexyl}- | | | | |
| | acetyl)piperidine-4-carbonitrile | | | | |
| 6H | 2-{trans-4-[4-(4-amino-5-oxo-7,8- | 53.5 nm | — | — | — |
| | dihydropyrimido[5,4- | (n = 1) | | | |
| | f][1,4]oxazepin-6(5H)- | | | | |
| | yl)phenyl]cyclohexyl}-N-methyl-N- | | | | |
| | (tetrahydro-2H-pyran-4- | | | | |
| | yl)acetamide | | | | |
| 6I | 4-amino-6-[4-(trans-4-{2-[4- | 57.8 nm | — | — | — |
| | (methoxymethyl)piperidin-1-yl]-2- | (n = 4) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6J | 2-{trans-4-[4-(4-amino-5-oxo-7,8- | 51.0 nm | — | — | — |
| | dihydropyrimido[5,4- | (n = 3) | | | |
| | f][1,4]oxazepin-6(5H)- | | | | |
| | yl)phenyl]cyclohexyl}-N-(2- | | | | |
| | methoxyethyl)-N- | | | | |
| | methylacetamide | | | | |
| 6K | 4-amino-6-(4-{trans-4-[2-(3- | 249 nm | — | — | — |
| | hydroxyazetidin-1-yl)-2-oxoethyl]- | (n = 3) | | | |
| | cyclohexyl}phenyl)-7,8-dihydro- | | | | |
| | pyrimido[5,4-f][1,4]oxazepin- | | | | |
| | 5(6H)-one | | | | |
| 6L | 4-amino-6-(4-{trans-4-[2-(4,4- | 105 nm | — | — | — |
| | difluoropiperidin-1-yl)-2-oxoethyl]- | (n = 3) | | | |
| | cyclohexyl}phenyl)-7,8-dihydro-- | | | | |
| | pyrimido[5,4-f][1,4]oxazepin- | | | | |
| | 5(6H)-one | | | | |
| 6M | 4-amino-6-[4-(trans-4-{2-[3- | 45.7 nm | — | — | — |
| | (hydroxymethyl)piperidin-1-yl]-2- | (n = 1) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6N | 2-{trans-4-[4-(4-amino-5-oxo-7,8- | 37.5 nm | — | — | — |
| | dihydropyrimido[5,4- | (n = 1) | | | |
| | f][1,4]oxazepin-6(5H)- | | | | |
| | yl)phenyl]cyclohexyl}-N-ethyl-N- | | | | |
| | (4-hydroxybutyl)acetamide | | | | |
| 6O | 4-amino-6-[4-(trans-4-{2-[(3R)-3- | 162 nm | — | — | — |
| | hydroxypiperidin-1-yl]-2-oxoethyl}- | (n = 3) | | | |
| | cyclohexyl)phenyl]-7,8-dihydro- | | | | |
| | pyrimido[5,4-f][1,4]oxazepin- | | | | |
| | 5(6H)-one | | | | |
| 6P | 4-amino-6-[4-(trans-4-{2-[4-(1, | 39.3 nm | — | — | — |
| | hydroxyethyl)piperidin-1-yl]-2- | (n = 1) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6Q | 4-amino-6-(4-{trans-4-[2-(3- | 240 nm | — | — | — |
| | methyl-5,6- | (n = 3) | | | |
| | dihydro[1,2,4]triazolo[4,3- | | | | |
| | a]pyrazin-7(8H)-yl)-2-oxoethyl]- | | | | |
| | cyclohexyl}phenyl)-7,8-dihydro- | | | | |
| | pyrimido[5,4-f][1,4]oxazepin- | | | | |
| | 5(6H)-one | | | | |
| 6R | 4-amino-6-[4-(trans-4-{2-[3-ethyl- | 17.5 nm | — | — | — |
| | 3-(hydroxymethyl)pyrrolidin-1-yl]- | (n = 1) | | | |
| | 2-oxoethyl}cyclohexyl)phenyl]-7,8- | | | | |
| | dihydropyrimido[5,4- | | | | |
| | f][1,4]oxazepin-5(6H)-one | | | | |
| 6S | N-[(3R)-1-(2-{trans-4-[4-(4-amino- | 192 nm | — | — | — |
| | 5-oxo-7,8-dihydropyrimido[5,4-f]- | (n = 3) | | | |
| | [1,4]oxazepin-6(5H)-yl)phenyl]- | | | | |
| | cyclohexyl}acetyl)pyrrolidin-3-yl]- | | | | |
| | acetamide | | | | |
| 6T | 4-amino-6-[4-(trans-4-{2-[3- | 125 nm | — | — | — |
| | (hydroxymethyl)pyrrolidin-1-yl]-2- | (n = 3) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6U | 2-{trans-4-[4-(4-amino-5-oxo-7,8- | 129 nm | — | — | — |
| | dihydropyrimido[5,4- | (n = 1) | | | |
| | f][1,4]oxazepin-6(5H)- | | | | |
| | yl)phenyl]cyclohexyl}-N-ethyl-N- | | | | |
| | (2-hydroxy-1,1-dimethylethyl)- | | | | |
| | acetamide | | | | |
| 6V | 2-{trans-4-[4-(4-amino-5-oxo-7,8- | 30.7 nm | — | — | — |
| | dihydropyrimido[5,4- | (n = 1) | | | |
| | f][1,4]oxazepin-6(5H)- | | | | |
| | yl)phenyl]cyclohexyl}-N-(2- | | | | |
| | hydroxyethyl)-N-pentylacetamide | | | | |
| 6W | 2-{trans-4-[4-(4-amino-5-oxo-7,8- | 77.7 nm | — | — | — |
| | dihydropyrimido[5,4- | (n = 1) | | | |
| | f][1,4]oxazepin-6(5H)- | | | | |
| | yl)phenyl]cyclohexyl}-N-methyl-N- | | | | |
| | (2-pyridin-2-ylethyl)-acetamide, | | | | |
| | trifluoroacetate salt | | | | |
| 6X | 4-amino-6-[4-(trans-4-{2-[2- | 100 nm | — | — | — |
| | (hydroxymethyl)pyrrolidin-1-yl]-2- | (n = 3) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6Y | 6-(4-{trans-4-[2-(4- | 167 nm | — | — | — |
| | acetylpiperazin-1-yl)-2- | (n = 3) | | | |
| | oxoethyl]cyclohexyl}phenyl)-4- | | | | |
| | amino-7,8-dihydropyrimido[5,4-f]- | | | | |
| | [1,4]oxazepin-5(6H)-one | | | | |
| 6Z | 2-{trans-4-[4-(4-amino-5-oxo-7,8- | 89.6 nm | — | — | — |
| | dihydropyrimido[5,4- | (n = 1) | | | |
| | f][1,4]oxazepin-6(5H)- | | | | |
| | yl)phenyl]cyclohexyl}-N-ethyl-N- | | | | |
| | [(1R)-2-hydroxy-1-methylethyl]- | | | | |
| | acetamide | | | | |
| 6AA | 4-amino-6-[4-(trans-4-{2-[(3R)-3- | 148 nm | — | — | — |
| | hydroxypyrrolidin-1-yl]-2- | (n = 3) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6AB | 4-amino-6-(4-{trans-4-[2-(4- | 54.7 nm | — | — | — |
| | ethoxypiperidin-1-yl)-2-oxoethyl]- | (n = 1) | | | |
| | cyclohexyl}phenyl)-7,8-dihydro- | | | | |
| | pyrimido[5,4-f][1,4]oxazepin- | | | | |
| | 5(6H)-one | | | | |
| 6AC | 3-(2-(trans-4-(4-(4-amino-5-oxo- | 178 nm | — | — | — |
| | 7,8-dihydropyrimido[5,4- | (n = 1) | | | |
| | f][1,4]oxazepin-6(5H)- | | | | |
| | yl)phenyl)cyclohexyl)-N- | | | | |
| | methylacetamido)-N,N- | | | | |
| | dimethylpropanamide | | | | |
| 6AD | 2-{trans-4-[4-(4-amino-5-oxo-7,8- | 42.1 nm | — | — | — |
| | dihydropyrimido[5,4- | (n = 1) | | | |
| | f][1,4]oxazepin-6(5H)- | | | | |
| | yl)phenyl]cyclohexyl}-N-(2,2- | | | | |
| | difluoropropyl)-N-(2-hydroxyethyl)- | | | | |
| | acetamide | | | | |
| 6AE | 4-amino-6-(4-{trans-4-[2-(2,2- | 75.5 nm | — | — | — |
| | dimethyl-3-oxopiperazin-1-yl)-2- | (n = 1) | | | |
| | oxoethyl]cyclohexyl}phenyl)-7,8- | | | | |
| | dihydropyrimido[5,4- | | | | |
| | f][1,4]oxazepin-5(6H)-one | | | | |
| 6AF | 2-{trans-4-[4-(4-amino-5-oxo-7,8- | 82.8 nm | — | — | — |
| | dihydropyrimido[5,4- | (n = 1) | | | |
| | f][1,4]oxazepin-6(5H)- | | | | |
| | yl)phenyl]cyclohexyl}-N-(1,4- | | | | |
| | dioxan-2-ylmethyl)-N-methyl- | | | | |
| | acetamide | | | | |
| 6AG | 2-{trans-4-[4-(4-amino-5-oxo-7,8- | 52.8 nm | — | — | — |
| | dihydropyrimido[5,4- | (n = 1) | | | |
| | f][1,4]oxazepin-6(5H)- | | | | |
| | yl)phenyl]cyclohexyl}-N-[(1S,2S)- | | | | |
| | 2-hydroxycyclohexyl]-N- | | | | |
| | methylacetamide | | | | |
| 6AH | 2-{trans-4-[4-(4-amino-5-oxo-7,8- | 67.0 nm | — | — | — |
| | dihydropyrimido[5,4- | (n = 1) | | | |
| | f][1,4]oxazepin-6(5H)- | | | | |
| | yl)phenyl]cyclohexyl}-N-(2- | | | | |
| | hydroxyethyl)-N-(2-methylbutyl)- | | | | |
| | acetamide | | | | |
| 6AI | 4-amino-6-(4-{trans-4-[2-(2- | 80.4 nm | — | — | — |
| | methylpyrrolidin-1-yl)-2-oxoethyl]- | (n = 1) | | | |
| | cyclohexyl}phenyl)-7,8-dihydro- | | | | |
| | pyrimido[5,4-f][1,4]oxazepin- | | | | |
| | 5(6H)-one | | | | |
| 6AJ | 2-{trans-4-[4-(4-amino-5-oxo-7,8- | 65.5 nm | — | — | — |
| | dihydropyrimido[5,4- | (n = 1) | | | |
| | f][1,4]oxazepin-6(5H)- | | | | |
| | yl)phenyl]cyclohexyl}-N-(2- | | | | |
| | hydroxyethyl)-N-propylacetamide | | | | |
| 6AK | 2-{trans-4-[4-(4-amino-5-oxo-7,8- | 18.1 nm | — | — | — |
| | dihydropyrimido[5,4- | (n = 1) | | | |
| | f][1,4]oxazepin-6(5H)- | | | | |
| | yl)phenyl]cyclohexyl}-N-(4- | | | | |
| | fluorobenzyl)-N-methylacetamide | | | | |
| 6AL | 4-amino-6-(4-{trans-4-[2-oxo-2-(5- | 105 nm | — | — | — |
| | oxo-1,4-diazepan-1-yl)ethyl]- | (n = 1) | | | |
| | cyclohexyl}phenyl)-7,8-dihydro- | | | | |
| | pyrimido[5,4-f][1,4]oxazepin- | | | | |
| | 5(6H)-one | | | | |
| 6AM | 4-amino-6-(4-{trans-4-[2-(3- | 139 nm | — | — | — |
| | fluoroazetidin-1-yl)-2- | (n = 3) | | | |
| | oxoethyl]cyclohexyl}phenyl)-7,8- | | | | |
| | dihydropyrimido[5,4- | | | | |
| | f][1,4]oxazepin-5(6H)-one | | | | |
| 6AN | 4-amino-6-[4-(trans-4-{2-[3- | 71.3 nm | — | — | — |
| | (hydroxymethyl)-3- | (n = 1) | | | |
| | methylpiperidin-1-yl]-2- | | | | |
| | oxoethyl}cyclohexyl)phenyl]-7,8- | | | | |
| | dihydropyrimido[5,4-f]- | | | | |
| | [1,4]oxazepin-5(6H)-one | | | | |
| 6AO | 1-({trans-4-[4-(4-amino-5-oxo-7,8- | 40.0 nm | — | — | — |
| | dihydropyrimido[5,4- | (n = 1) | | | |
| | f][1,4]oxazepin-6(5H)- | | | | |
| | yl)phenyl]cyclohexyl}- | | | | |
| | acetyl)piperidine-3-carbonitrile | | | | |
| 6AP | 4-amino-6-(4-{trans-4-[2-(4- | 107 nm | — | — | — |
| | hydroxy-4-methylpiperidin-1-yl)-2- | (n = 1) | | | |
| | oxoethyl]-cyclohexyl}phenyl)-7,8- | | | | |
| | dihydro-pyrimido[5,4- | | | | |
| | f][1,4]oxazepin-5(6H)-one | | | | |
| 6AQ | 2-{trans-4-[4-(4-amino-5-oxo-7,8- | 87.5 nm | — | — | — |
| | dihydropyrimido[5,4- | (n = 1) | | | |
| | f][1,4]oxazepin-6(5H)- | | | | |
| | yl)phenyl]cyclohexyl}-N-methyl-N- | | | | |
| | (1-pyridin-3-ylethyl)-acetamide, | | | | |
| | trifluoroacetate salt | | | | |
| 6AR | 4-amino-6-(4-{trans-4-[2-(3- | 85.3 nm | — | — | — |
| | hydroxy-3-methylpyrrolidin-1-yl)-2- | (n = 1) | | | |
| | oxoethyl]cyclohexyl}phenyl)-7,8- | | | | |
| | dihydropyrimido[5,4- | | | | |
| | f][1,4]oxazepin-5(6H)-one | | | | |
| 6AS | 2-{trans-4-[4-(4-amino-5-oxo-7,8- | 47.4 nm | — | — | — |
| | dihydropyrimido[5,4- | (n = 1) | | | |
| | f][1,4]oxazepin-6(5H)- | | | | |
| | yl)phenyl]cyclohexyl}-N-ethyl-N- | | | | |
| | [(1-methyl-1H-imidazol-2- | | | | |
| | yl)methyl]acetamide, | | | | |
| | trifluoroacetate salt | | | | |
| 6AT | N-2-({trans-4-[4-(4-amino-5-oxo- | 161 nm | — | — | — |
| | 7,8-dihydropyrimido[5,4-f]- | (n = 3) | | | |
| | [1,4]oxazepin-6(5H)-yl)phenyl]- | | | | |
| | cyclohexyl}acetyl)-N,N,N-2- | | | | |
| | trimethylglycinamide | | | | |
| 6AU | 2-{trans-4-[4-(4-amino-5-oxo-7,8- | 75.5 nm | — | — | — |
| | dihydropyrimido[5,4- | (n = 1) | | | |
| | f][1,4]oxazepin-6(5H)- | | | | |
| | yl)phenyl]cyclohexyl}-N-methyl-N- | | | | |
| | (1-pyridin-4-ylethyl)acetamide, | | | | |
| | trifluoroacetate salt | | | | |
| 6AV | 4-amino-6-(4-{trans-4-[2-(4- | 170 nm | — | — | — |
| | methyl-3-oxopiperazin-1-yl)-2- | (n = 3) | | | |
| | oxoethyl]-cyclohexyl}phenyl)-7,8- | | | | |
| | dihydro-pyrimido[5,4- | | | | |
| | f][1,4]oxazepin-5(6H)-one | | | | |
| 6AW | 4-amino-6-{4-[trans-4-(2-azetidin- | 73.2 nm | — | — | — |
| | 1-yl-2- | (n = 3) | | | |
| | oxoethyl)cyclohexyl]phenyl}-7,8- | | | | |
| | dihydropyrimido[5,4-f]- | | | | |
| | [1,4]oxazepin-5(6H)-one | | | | |
| 6AX | 4-amino-6-(4-{trans-4-[2-oxo-2-(3- | 123 nm | — | — | — |
| | oxopiperazin-1- | (n = 3) | | | |
| | yl)ethyl]cyclohexyl}-phenyl)-7,8- | | | | |
| | dihydropyrimido[5,4-f]- | | | | |
| | [1,4]oxazepin-5(6H)-one | | | | |
| 6AY | 4-amino-6-[4-(trans-4-{2-[2-(2- | 85.3 nm | — | — | — |
| | hydroxyethyl)piperidin-1-yl]-2- | (n = 1) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6AZ | 2-{trans-4-[4-(4-amino-5-oxo-7,8- | 67.0 nm | — | — | — |
| | dihydropyrimido[5,4- | (n = 1) | | | |
| | f][1,4]oxazepin-6(5H)- | | | | |
| | yl)phenyl]cyclohexyl}-N-methyl-N- | | | | |
| | [(2-methylpyridin-4- | | | | |
| | yl)methyl]acetamide, | | | | |
| | trifluoroacetate salt | | | | |
| 6BA | 4-amino-6-[4-(trans-4-{2-[(3R)-3- | 60.1 nm | — | — | — |
| | methoxypyrrolidin-1-yl]-2- | (n = 1) | | | |
| | oxoethyl}cyclohexyl)phenyl]-7,8- | | | | |
| | dihydropyrimido[5,4- | | | | |
| | f][1,4]oxazepin-5(6H)-one | | | | |
| 6BB | 4-amino-6-(4-{trans-4-[2-(2- | 147 nm | — | — | — |
| | methyl-3-oxopiperazin-1-yl)-2- | (n = 3) | | | |
| | oxoethyl]-cyclohexyl}phenyl)-7,8- | | | | |
| | dihydro-pyrimido[5,4- | | | | |
| | f][1,4]oxazepin-5(6H)-one | | | | |
| 6BC | 4-amino-6-(4-{trans-4-[2-(3- | 104 nm | — | — | — |
| | hydroxypyrrolidin-1-yl)-2- | (n = 3) | | | |
| | oxoethyl]cyclohexyl}phenyl)-7,8- | | | | |
| | dihydropyrimido[5,4- | | | | |
| | f][1,4]oxazepin-5(6H)-one | | | | |
| 6BD | 4-amino-6-[4-(trans-4-{2-[(3R)-3- | 47.2 nm | — | — | — |
| | methylmorpholin-4-yl]-2-oxoethyl}- | (n = 1) | | | |
| | cyclohexyl)phenyl]-7,8-dihydro- | | | | |
| | pyrimido[5,4-f][1,4]oxazepin- | | | | |
| | 5(6H)-one | | | | |
| 6BE | 4-amino-6-[4-(trans-4-{2-[(3S)-3- | 95.1 nm | — | — | — |
| | hydroxypyrrolidin-1-yl]-2- | (n = 3) | | | |
| | oxoethyl}-cyclohexyl)phenyl]-7,8- | | | | |
| | dihydro-pyrimido[5,4- | | | | |
| | f][1,4]oxazepin-5(6H)-one | | | | |
| 6BF | 4-amino-6-{4-[trans-4-(3- | 60.1 nm | — | — | — |
| | morpholin-4-yl-3- | (n = 1) | | | |
| | oxopropyl)cyclohexyl]phenyl}-7,8- | | | | |
| | dihydropyrimido[5,4- | | | | |
| | f][1,4]oxazepin-5(6H)-one | | | | |
| 6BG | 4-[(8R)-4-amino-8-methyl-5-oxo- | — | — | — | 1670 nm |
| | 7,8-dihydropyrimido[5,4- | | | | (n = 1) |
| | f][1,4]oxazepin-6(5H)-yl]-N,N- | | | | |
| | dimethylbenzamide | | | | |
| 7A | 6-[4-(1-acetylpiperidin-4- | — | 119 nm | 234 nm | — |
| | yl)phenyl]-4-amino-7,8- | | (n = 2) | (n = 2) | |
| | dihydropyrimido[5,4-f]- | | | | |
| | [1,4]oxazepin-5(6H)-one | | | | |
| 7B | t-butyl 4-[4-(4-amino-5-oxo-7,8- | — | 55.1 nm | 30.0 nm | — |
| | dihydro-pyrimido[5,4-f]- | | (n = 1) | (n = 2) | |
| | [1,4]oxazepin-6(5H)- | | | | |
| | yl)phenyl]piperidine-1-carboxylate | | | | |
| 7C | 4-amino-6-{4-[1-(1,3-thiazol-4- | — | 82.7 nm | 116 nm | — |
| | ylacetyl)piperidin-4-yl]phenyl}-7,8- | | (n = 2) | (n = 2) | |
| | dihydropyrimido[5,4-f][1, | | | | |
| | 4]oxazepin-5(6H)-one | | | | |
| 7D | 4-amino-6-(4-{1-[(3S)-3- | — | 98.2 nm | 267 nm | — |
| | hydroxybutanoyl]piperidin-4- | | (n = 2) | (n = 2) | |
| | yl}phenyl)-7,8- | | | | |
| | dihydropyrimido[5,4-f][1, | | | | |
| | 4]oxazepin-5(6H)-one | | | | |
| 7E | 4-amino-6-{4-[1-(tetrahydro-2H- | — | 196 nm | 300 nm | — |
| | pyran-3-ylacetyl)piperidin-4- | | (n = 2) | (n = 2) | |
| | yl]phenyl}-7,8- | | | | |
| | dihydropyrimido[5,4- | | | | |
| | f][1,4]oxazepin-5(6H)-one | | | | |
| 7F | 4-amino-6-{4-[1-(tetrahydrofuran- | — | 102 nm | 247 nm | — |
| | 3-ylcarbonyl)piperidin-4- | | (n = 2) | (n = 2) | |
| | yl]phenyl}-7,8- | | | | |
| | dihydropyrimido[5,4-f][1, | | | | |
| | 4]oxazepin-5(6H)-one | | | | |
| 7G | 4-amino-6-(4-{1-[(5-methyl-1,3- | — | 88.2 nm | 290 nm | — |
| | oxazol-4-yl)carbonyl]piperidin-4- | | (n = 2) | (n = 2) | |
| | yl}phenyl)-7,8- | | | | |
| | dihydropyrimido[5,4-f]- | | | | |
| | [1,4]oxazepin-5(6H)-one | | | | |
| 7H | 4-amino-6-{4-[1-(tetrahydro-2H- | — | 143 nm | 215 nm | — |
| | pyran-4-ylcarbonyl)piperidin-4- | | (n = 2) | (n = 2) | |
| | yl]phenyl}-7,8- | | | | |
| | dihydropyrimido[5,4-f]- | | | | |
| | [1,4]oxazepin-5(6H)-one | | | | |
| 7I | 4-amino-6-{4-[1-(1,2,5-oxadiazol- | — | 15.3 nm | 8.62 nm | — |
| | 3-ylcarbonyl)piperidin-4- | | (n = 2) | (n = 2) | |
| | yl]phenyl}-7,8- | | | | |
| | dihydropyrimido[5,4- | | | | |
| | f][1,4]oxazepin-5(6H)-one | | | | |
| 7J | 4-amino-6-(4-{1-[(cis-4- | — | 70.1 nm | 113 nm | — |
| | hydroxycyclohexyl)carbonyl]- | | (n = 2) | (n = 2) | |
| | piperidin-4-yl}phenyl)-7,8- | | | | |
| | dihydropyrimido[5,4-f]- | | | | |
| | [1,4]oxazepin-5(6H)-one | | | | |
| 7K | 4-amino-6-(4-{1-[(4-methyl-1,3- | — | 130 nm | 258 nm | — |
| | oxazol-5-yl)carbonyl]piperidin-4- | | (n = 2) | (n = 2) | |
| | yl}phenyl)-7,8-dihydropyrimido | | | | |
| | [5,4-f][1,4]oxazepin-5(6H)-one | | | | |
| 7L | 4-amino-6-{4-[1-(isoxazol-3-yl- | — | 64.9 nm | 174 nm | — |
| | carbonyl)piperidin-4-yl]phenyl}- | | (n = 2) | (n = 2) | |
| | 7,8-dihydropyrimido[5,4-f][1, | | | | |
| | 4]oxazepin-5(6H)-one | | | | |
| 7M | 4-amino-6-{4-[1-(tetrahydrofuran- | — | 114 nm | 180 nm | — |
| | 3-ylacetyl)piperidin-4-yl]phenyl}- | | (n = 2) | (n = 2) | |
| | 7,8-dihydropyrimido[5,4-f][1, | | | | |
| | 4]oxazepin-5(6H)-one | | | | |
| 7N | 4-amino-6-{4-[1-(pyrimidin-2- | — | 119 nm | 234 nm | — |
| | ylacetyl)piperidin-4-yl]phenyl}-7,8- | | (n = 2) | (n = 2) | |
| | dihydropyrimido[5,4-f][1, | | | | |
| | 4]oxazepin-5(6H)-one | | | | |
| 7O | 4-amino-6-{4-[1-(tetrahydro-2H- | — | 138 nm | 374 nm | — |
| | pyran-4-ylacetyl)piperidin-4- | | (n = 2) | (n = 2) | |
| | yl]phenyl}-7,8- | | | | |
| | dihydropyrimido[5,4-f][1, | | | | |
| | 4]oxazepin-5(6H)-one | | | | |
| 7P | 4-amino-6-{4-[1-(isoxazol-5- | — | 34.6 nm | 79.1 | — |
| | ylcarbonyl)piperidin-4-yl]phenyl}- | | (n = 2) | (n = 2) | |
| | 7,8-dihydropyrimido[5,4-f][1, | | | | |
| | 4]oxazepin-5(6H)-one | | | | |
| 7Q | 4-amino-6-{4-[1- | — | 147 nm | 274 nm | — |
| | (ethoxyacetyl)piperidin-4- | | (n = 2) | (n = 2) | |
| | yl]phenyl}-7,8- | | | | |
| | dihydropyrimido[5,4-f]- | | | | |
| | [1,4]oxazepin-5(6H)-one | | | | |
| 7R | 4-amino-6-{4-[1-(tetrahydrofuran- | — | 50.1 nm | 170 nm | — |
| | 2-yl-acetyl)piperidin-4-yl]phenyl}- | | (n = 2) | (n = 2) | |
| | 7,8-dihydropyrimido[5,4-f][1, | | | | |
| | 4]oxazepin-5(6H)-one | | | | |
| 7S | 4-amino-6-[4-(1- | — | 82.2 nm | 174 nm | — |
| | isobutyrylpiperidin-4-yl)phenyl]- | | (n = 2) | (n = 3) | |
| | 7,8-dihydropyrimido[5,4-f]- | | | | |
| | [1,4]oxazepin-5(6H)-one | | | | |
| 7T | 4-amino-6-(4-{1-[(methylsulfonyl)- | — | 123 nm | 217 nm | — |
| | acetyl]piperidin-4-yl}phenyl)-7,8- | | (n = 2) | (n = 3) | |
| | dihydropyrimido[5,4-f][1, | | | | |
| | 4]oxazepin-5(6H)-one | | | | |
| 7U | 4-amino-6-(4-{1-[(2S)-2-hydroxy- | — | 205 nm | 297 nm | — |
| | 3-methylbutanoyl]piperidin-4- | | (n = 2) | (n = 3) | |
| | yl}phenyl)-7,8- | | | | |
| | dihydropyrimido[5,4-f][1, | | | | |
| | 4]oxazepin-5(6H)-one | | | | |
| 7V | 4-amino-6-(4-{1-[(5- | — | 113 nm | 224 nm | — |
| | methylisoxazol-3- | | (n = 2) | (n = 2) | |
| | yl)carbonyl]piperidin-4-yl}phenyl)- | | | | |
| | 7,8-dihydropyrimido[5,4-f][1, | | | | |
| | 4]oxazepin-5(6H)-one | | | | |
| 7W | 4-amino-6-{4-[1-(tetrahydro-2H- | — | — | — | — |
| | pyran-2-ylacetyl)piperidin-4- | | | | |
| | yl]phenyl}-7,8- | | | | |
| | dihydropyrimido[5,4-f][1, | | | | |
| | 4]oxazepin-5(6H)-one | | | | |
| 7X | 4-amino-6-{4-[1-(tetrahydrofuran- | — | 88.4 nm | 157 nm | — |
| | 2-ylcarbonyl)piperidin-4- | | (n = 2) | (n = 2) | |
| | yl]phenyl}-7,8- | | | | |
| | dihydropyrimido[5,4-f][1, | | | | |
| | 4]oxazepin-5(6H)-one | | | | |
| 7Y | 4-amino-6-{4-[1- | — | 45.7 nm | 98.6 nm | — |
| | (phenylacetyl)piperidin-4- | | (n = 2) | (n = 2) | |
| | yl]phenyl}-7,8- | | | | |
| | dihydropyrimido[5,4-f]- | | | | |
| | [1,4]oxazepin-5(6H)-one | | | | |
| 7Z | 4-amino-6-{4-[1-(tetrahydro-2H- | — | 105 nm | 134 nm | — |
| | pyran-3-ylcarbonyl)piperidin-4- | | (n = 2) | (n = 2) | |
| | yl]phenyl}-7,8- | | | | |
| | dihydropyrimido[5,4-f][1, | | | | |
| | 4]oxazepin-5(6H)-one | | | | |
| 7AA | 4-amino-6-{4-[1- | — | 48.9 nm | 85.3 nm | — |
| | (cyclohexylcarbonyl)-piperidin-4- | | (n = 2) | (n = 2) | |
| | yl]phenyl}-7,8- | | | | |
| | dihydropyrimido[5,4-f][1, | | | | |
| | 4]oxazepin-5(6H)-one | | | | |
| 7AB | 4-amino-6-{4-[1-(1H-1,2,4-triazol- | — | 188 nm | 281 nm | — |
| | 1-yl-acetyl)piperidin-4-yl]phenyl}- | | (n = 2) | (n = 2) | |
| | 7,8-dihydropyrimido[5,4-f][1, | | | | |
| | 4]oxazepin-5(6H)-one | | | | |
| 7AC | 4-amino-6-{4-[1-(1H-pyrazol-1- | — | 73.1 nm | 113 nm | — |
| | ylacetyl)piperidin-4-yl]phenyl}-7,8- | | (n = 2) | (n = 2) | |
| | dihydropyrimido[5,4-f][1, | | | | |
| | 4]oxazepin-5(6H)-one | | | | |
| 7AD | 4-amino-6-[4-(1-benzoylpiperidin- | — | 43.0 nm | 88.3 nm | — |
| | 4-yl)phenyl]-7,8- | | (n = 2) | (n = 2) | |
| | dihydropyrimido[5,4-f]- | | | | |
| | [1,4]oxazepin-5(6H)-one | | | | |
| 7AE | 4-amino-6-{4-[1- | — | 41.2 nm | 92.3 nm | — |
| | (cyclopentylacetyl)-piperidin-4- | | (n = 2) | (n = 2) | |
| | yl]phenyl}-7,8- | | | | |
| | dihydropyrimido[5,4-f][1, | | | | |
| | 4]oxazepin-5(6H)-one | | | | |

The following assays may also be used to further define the utility of the compounds of the present invention.

### In Vivo Assay for Glucose Lowering

Oral glucose tolerance tests (“OGTT”) have been in use in humans since, at least, the 1930s, Pincus et al., Am J Med Sci 188, 782 (1934), and are routinely used in the diagnosis of human diabetes, though not to evaluate the efficacy of therapeutic agents in patients.

KK mice have been used to evaluate glitazones (Fujita, et al., Diabetes. 32, 804-810 (1983); Fujiwara, et al., Diabetes. 37, 1549-48 (1988); Izumi et al. Biopharm Drug Dispos, 18, 247-257 (1997), metformin (Reddi, et al., Diabet Metabl. 19, 44-51 (1993), glucosidase inhibitors (Hamada, et al., Jap Pharmacol Ther, 17, 17-28 (1988); Matsuo, et al., Am J Clin Nutr, 55, 314S-317S (1992)), and the extra-pancreatic effects of sulfonylureas (Kameda, et al., Arzenim Forsch./Drug Res. 32, 39044 (1982); and Muller, et al., Horm Metabl Res, 28, 469-487 (1990)).

KK mice are derived from an inbred line first established by Kondo et al. (Kondo, et al., Bull Exp Anim, 6, 107-112 (1957)). The mice spontaneously develop a hereditary form of polygenic diabetes that progresses to cause renal, retinal and neurological complications analogous to those seen in human diabetic subjects, but they do not require insulin or other medication for survival. Another aspect of the invention is directed to the use of KK mice to evaluate the effects of insulin secretagogue agents in the context of an oral glucose tolerance test.

### In Vivo Assay for Food Intake

The following screen may be used to evaluate the efficacy of test compounds for inhibiting food intake in Sprague-Dawley rats after an overnight fast.

Male Sprague-Dawley rats are individually housed and fed powdered chow. They are maintained on a 12 hour light/dark cycle and received food and water ad libitum. The animals are acclimated to the vivarium for a period of one week before testing is conducted. Testing is completed during the light portion of the cycle.

To conduct the food intake efficacy screen, rats are transferred to individual test cages without food the afternoon prior to testing, and the rats are fasted overnight. After the overnight fast, rats are dosed the following morning with vehicle or test compounds. A known antagonist is dosed (3 mg/kg) as a positive control, and a control group receives vehicle alone (no compound). The test compounds are dosed at ranges between 0.1 and 100 mg/kg depending upon the compound. The standard vehicle is 0.5% (w/v) methylcellulose in water and the standard route of administration is oral. However, different vehicles and routes of administration may be used to accommodate various compounds when required. Food is provided to the rats 30 minutes after dosing and an Oxymax automated food intake system (Columbus Instruments, Columbus, Ohio) is started. Individual rat food intake is recorded continuously at 10-minute intervals for a period of two hours. When required, food intake is recorded manually using an electronic scale; food is weighed every 30 minutes after food is provided up to four hours after food is provided. Compound efficacy is determined by comparing the food intake pattern of compound-treated rats to vehicle and the standard positive control.

Throughout this application, various publications are referenced. The disclosures of these publications in their entireties are hereby incorporated by reference into this application for all purposes.

It will be apparent to those skilled in the art that various modifications and variations can be made in the invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims and the application as a whole.

What is claimed is:

1. A compound having Formula (I) 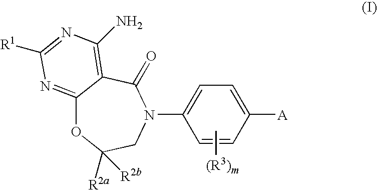
wherein
R¹ is hydrogen, (C₁-C₄)alkyl, (C₁-C₄)perfluoroalkyl, (C₁-C₄)perfluoroalkoxy, or (C₁-C₄)alkoxy;
R^{2a} and R^{2b}, taken separately, are each independently hydrogen, (C₁-C₄)alkyl, or (C₁-C₄)perfluoroalkyl, or R^{2a} and R^{2b}, taken together, are (C₃-C₆)cycloalkyl;
m is 0, 1 or 2;
R³ is halo, (C₁-C₄)alkyl, (C₃-C₆)cycloalkyl, (C₁-C₄)alkoxy, hydroxyl or CF₃, when m is 2, R³ can be the same or different and when m is 0, R³ is hydrogen;
A is a chemical moiety selected from the group consisting of
(i) (C₁-C₆)alkyl optionally substituted with one or two substituents selected from the group consisting of —N(R⁵)(R⁶), hydroxyl, (C₁-C₄)alkoxy, (C₁-C₄)haloalkyl, halo, cyano, —C(O)—OH, —C(O)—(C₁-C₄)alkoxy, and —C(O)—N(R⁵)(R⁶);
(ii) halo;
(iii) 3- to 5-membered carbocyclic ring optionally substituted with hydroxy, (C₁-C₄)alkoxy, cyano or 1 to 2 halo groups;
(iv) —C(O)—R⁴;
(v) a group of formula (Ia) 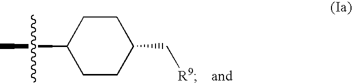
(vi) a group of formula (Ib) 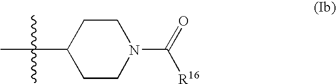
R⁴ is —OR⁵ or —N(R⁵)(R⁶);
R⁵ and R⁶ are each independently selected from H or (C₁-C₆)alkyl;
R⁹ is
(a) —(CH₂)ₚ—C(O)—N(R^{10a})(R^{10b}), where p is 0 or 1, R^{10a} is (C₁-C₆)alkyl-, or halo-substituted(C₁-C₃)alkyl-, and R^{10b} is —CH(CH₃)—R^{10c} or —(CH₂)_{q}R^{10c}, where q is 0, 1 or 2 and R^{10c} is (C₁-C₄)alkyl, —C(O)OH, —C(O)N((C₁-C₃)alkyl)₂, —C(O)NH(C₁-C₃)alkyl, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, (C₁-C₃)alkyl, (C₁-C₄)alkoxy, or cyano;
or R^{10a} and R^{10b} taken together with the nitrogen to which they are attached form a 4- to 7-membered heterocycle optionally containing an additional heteroatom selected from oxygen, nitrogen or sulfur, where said heterocycle is optionally fused to a 5- to 6-membered heteroaryl containing 1 to 3 heteroatoms each independently selected from O, N or S, wherein said heterocycle and said fused heterocycle are optionally substituted with 1 to 3 substituents selected from hydroxyl, cyano, halo, (C₁-C₃)alkoxy-, (C₁-C₃)alkyl-, hydroxy(C₁-C₆)alkyl-, (C₁-C₃)alkoxy(C₁-C₃)alkyl-, CH₃C(O)NH—, CH₃C(O)—, or oxo;
(b) —(CH₂)ᵣ—R¹¹, where r is 0, 1 or 2 and R¹¹ is a chemical moiety selected from the group consisting of 1,3-thiazol-4-yl, 1,2,4-oxadiazol-5-yl, 1,3,4-oxadiazol-2-yl, 1,2,4-triazol-3-yl, 1,2,5-triazol-3-yl, or 1,3,4-thiadazol-2-yl; wherein said chemical moiety is optionally substituted with 1 to 3 (C₁-C₃)alkyl groups;
(c) —(CH₂)ₛC(OH)(R¹²)(R¹³), where s is 0, 1, or 2 and R¹² and R¹³ are each independently a H or (C₁-C₃)alkyl; or
(d) —(CH₂)ₜ—C(NH₂)(R¹⁴)(R¹⁵), where t is 0, 1, or 2 and R¹⁴ and R¹⁵ are each independently a H or (C₁-C₃)alkyl; and
R¹⁶ is (C₁-C₆)alkyl optionally substituted with hydroxyl, (C₁-C₃)alkoxy, (C₁-C₃)alkyl-SO₂—, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, or (C₁-C₃)alkyl;
or a pharmaceutically acceptable salt thereof.

2. A compound having Formula (I*) 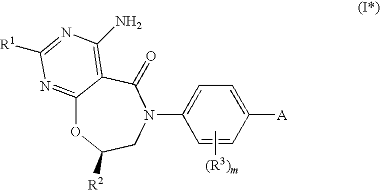
wherein
R¹ is hydrogen, (C₁-C₃)alkyl, methoxy or halo-substituted (C₁-C₃)alkyl;
R² is hydrogen or methyl;
m is 0, 1 or 2;
R³ is halo, methyl, methoxy, or CF₃, when m is 2, R³ can be the same or different and when m is 0, R³ is hydrogen;
A is a chemical moiety selected from the group consisting of
(i) (C₁-C₆)alkyl;
(ii) 3- to 5-membered carbocyclic ring optionally substituted with hydroxy, (C₁-C₄)alkoxy, cyano or 1 to 2 halo groups;
(iii) —C(CH₃)₂—R⁴, where R⁴ is cyano, hydroxyl, —C(O)NH₂, —C(O)—O(C₁-C₃)alkyl, —CH₂OH, or fluoro;
(iv) —C(O)O(C₁-C₃)alkyl;
(v) —C(O)—N(R⁵)(R⁶), where R⁵ and R⁶ are each independently selected from H or (C₁-C₃)alkyl;
(vi) —(CH₂)ₙ—C(OH)(R⁷)(R⁸), where n is 0 or 1 and R⁷ and R⁸ are each independently a H, (C₁-C₃)alkyl, or —CF₃;
(vii) taken together with R³ on an adjacent carbon to form a 5- to 6-membered carbocyclic fused ring;
(viii) a group of formula (Ia) 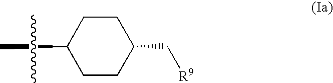
wherein R⁹ is
(a) —(CH₂)ₚ—C(O)—N(R^{10a})(R^{10b}), where p is 0 or 1, R^{10a} is (C₁-C₆)alkyl-, or halo-substituted(C₁-C₃)alkyl-, and R^{10b} is —CH(CH₃)—R^{10c} or —(CH₂)_{q}R^{10c}, where q is 0, 1 or 2 and R^{10c} is (C₁-C₄)alkyl, —C(O)OH, —C(O)N((C₁-C₃)alkyl)₂, —C(O)NH(C₁-C₃)alkyl, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, (C₁-C₃)alkyl, (C₁-C₄)alkoxy, or cyano;
or R^{10a} and R^{10b} taken together with the nitrogen to which they are attached form a 4- to 7-membered heterocycle optionally containing an additional heteroatom selected from oxygen, nitrogen or sulfur, where said heterocycle is optionally fused to a 5- to 6-membered heteroaryl containing 1 to 3 heteroatoms each independently selected from O, N or S, wherein said heterocycle and said fused heterocycle are optionally substituted with 1 to 3 substituents selected from hydroxyl, cyano, halo, (C₁-C₃)alkoxy-, (C₁-C₃)alkyl-, hydroxy(C₁-C₆)alkyl-, (C₁-C₃)alkoxy(C₁-C₃)alkyl-, CH₃C(O)NH—, CH₃C(O)—, or oxo;
(b) —(CH₂)ᵣ—R¹¹, where r is 0, 1 or 2 and R¹¹ is a chemical moiety selected from the group consisting of 1,3-thiazol-4-yl, 1,2,4-oxadiazol-5-yl, 1,3,4-oxadiazol-2-yl, 1,2,4-triazol-3-yl, 1,2,5-triazol-3-yl, or 1,3,4-thiadazol-2-yl; wherein said chemical moiety is optionally substituted with 1 to 3 (C₁-C₃)alkyl groups;
(c) —(CH₂)ₛ—C(OH)(R¹²)(R¹³), where s is 0, 1, or 2 and R¹² and R¹³ are each independently a H or (C₁-C₃)alkyl; or
(d) —(CH₂)ₜ—C(NH₂)(R¹⁴)(R¹⁵), where t is 0, 1, or 2 and R¹⁴ and R¹⁵ are each independently a H or (C₁-C₃)alkyl; and
(ix) a group of formula (Ib) 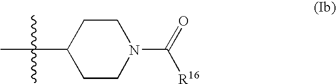
wherein R¹⁶ is
(a) —CH(CH₃)—R¹⁷ or —(CH₂)ᵥR¹⁷, where v is 0, 1 or 2 and R¹⁷ is hydrogen, (C₁-C₃)alkyl, (C₁-C₃)alkoxy, (C₁-C₃)alkyl-SO₂—, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, or (C₁-C₃)alkyl; or
(b) —(CH₂)_{w}—C(OH)(R¹⁸)(R¹⁹), where w is 0 or 1 and R¹⁸ and R¹⁹ are each independently a H or (C₁-C₃)alkyl;
or a pharmaceutically acceptable salt thereof.

3. The compound of claim 1 or 2 wherein A is a chemical moiety selected from the group consisting of
(i) (C₁-C₆)alkyl;
(ii) 3- to 5-membered carbocyclic ring optionally substituted with hydroxy, (C₁-C₄)alkoxy, cyano or 1 to 2 halo groups;
(iii) —C(CH₃)₂—R⁴, where R⁴ is cyano, hydroxyl, —C(O)NH₂, —C(O)—O(C₁-C₃)alkyl, —CH₂OH, or fluoro;
(iv) —C(O)O(C₁-C₃)alkyl;
(v) —C(O)—N(R⁵)(R⁶), where R⁵ and R⁶ are each independently selected from H or (C₁-C₃)alkyl;
(vi) —(CH₂)ₙ—C(OH)(R⁷)(R⁸), where n is 0 or 1 and R⁷ and R⁸ are each independently a H, (C₁-C₃)alkyl, or —CF₃; and
(vii) taken together with R³ on an adjacent carbon to form a 5- to 6-membered carbocyclic fused ring;
or a pharmaceutically acceptable salt thereof.

4. The compound of claim 1 wherein
R¹ is hydrogen or methoxy;
R² is methyl or hydrogen;
m is 0 or 1;
A is (i) (C₁-C₆)alkyl optionally substituted with one or two substituents selected from the group consisting of (C₁-C₄)haloalkyl, —C(O)—OH, —C(O)—(C₁-C₄)alkoxy, and —C(O)—N(R⁵)(R⁶); or
(ii) halo;
or a pharmaceutically acceptable salt thereof.

5. A compound selected from the group consisting of:
(8R)-4-amino-8-methyl-6-(4-methylphenyl)-7,8-dihydropyrimido[5,4-f]-[1,4]oxazepin-5(6H)-one;
(8R)-4-amino-6-[4-(cis-3-hydroxycyclobutyl)phenyl]-8-methyl-7,8-dihydropyrimido[5,4-f][1,4]oxazepin-5(6H)-one;
(8R)-4-amino-6-(4-tert-butylphenyl)-2-methoxy-8-methyl-7,8-dihydropyrimido[5,4-f][1,4]oxazepin-5(6H)-one;
(8R)-4-amino-6-[4-(1-methoxycyclobutyl)phenyl]-8-methyl-7,8-dihydropyrimido[5,4-f][1,4]oxazepin-5(6H)-one;
(8R)-4-amino-6-[4-(3,3-difluorocyclobutyl)phenyl]-8-methyl-7,8-dihydropyrimido[5,4-f][1,4]oxazepin-5(6H)-one;
(8R)-4-amino-6-(4-isobutylphenyl)-8-methyl-7,8-dihydropyrimido[5,4-f]-[1,4]oxazepin-5(6H)-one;
(8R)-4-amino-6-(4-ethylphenyl)-8-methyl-7,8-dihydropyrimido[5,4-f]-[1,4]oxazepin-5(6H)-one;
(8R)-4-amino-6-(4-tert-butylphenyl)-8-methyl-7,8-dihydropyrimido[5,4-f]-[1,4]oxazepin-5(6H)-one;
(8R)-4-amino-6-(4-isopropylphenyl)-8-methyl-7,8-dihydropyrimido[5,4-f]-[1,4]oxazepin-5(6H)-one;
(8R)-4-amino-6-(4-cyclopropylphenyl)-8-methyl-7,8-dihydropyrimido[5,4-f][1,4]oxazepin-5(6H)-one;
4-amino-6-(4-tert-butylphenyl)-7,8-dihydropyrimido[5,4-f][1,4]oxazepin-5(6H)-one;
(8R)-4-amino-6-(2,3-dihydro-1H-inden-5-yl)-8-methyl-7,8-dihydropyrimido-[5,4-f][1,4]oxazepin-5(6H)-one;
(8R)-4-amino-8-methyl-6-[4-(2,2,2-trifluoro-1,1-dimethylethyl)phenyl]-7,8-dihydropyrimido[5,4-f][1,4]oxazepin-5(6H)-one;
(8R)-4-amino-6-(3,4-dichlorophenyl)-8-methyl-7,8-dihydropyrimido[5,4-f][1,4]oxazepin-5(6H)-one;
2-{4-[(8R)-4-amino-8-methyl-5-oxo-7,8-dihydropyrimido[5,4-f][1,4]oxazepin-6(5H)-yl]phenyl}-2-methylpropanoic acid; and
2-{4-[(8R)-4-amino-8-methyl-5-oxo-7,8-dihydropyrimido[5,4-f][1,4]oxazepin-6(5H)-yl]phenyl}-2-methylpropanamide
or a pharmaceutically acceptable salt thereof.

6. The compound of claim 1 or 2 having Formula (II) 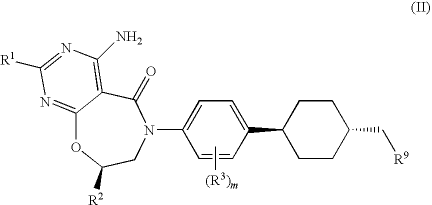
wherein
R¹ is hydrogen, (C₁-C₃)alkyl, methoxy or halo-substituted (C₁-C₃)alkyl;
R² is hydrogen or methyl;
m is 0, 1 or 2;
R³ is halo, methyl, methoxy, or CF₃, when m is 2, R³ can be the same or different;
R⁹ is selected from the group consisting of
(i) —(CH₂)ₚ—C(O)—N(R^{10a})(R^{10b}), where p is 0 or 1, R^{10a} is (C₁-C₆)alkyl-, or halo-substituted(C₁-C₃)alkyl-, and R^{10b} is —CH(CH₃)—R^{10c} or —(CH₂)_{q}R^{10c}, where q is 0, 1 or 2 and R^{10c} is (C₁-C₄)alkyl, —C(O)OH, —C(O)N((C₁-C₃)alkyl)₂, —C(O)NH(C₁-C₃)alkyl, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, (C₁-C₃)alkyl, (C₁-C₄)alkoxy, or cyano;
or R^{10a} and R^{10b} taken together with the nitrogen to which they are attached form a 4- to 7-membered heterocycle optionally containing an additional heteroatom selected from oxygen, nitrogen or sulfur, where said heterocycle is optionally fused to a 5- to 6-membered heteroaryl containing 1 to 3 heteroatoms each independently selected from O, N or S, wherein said heterocycle and said fused heterocycle are optionally substituted with 1 to 3 substituents selected from hydroxyl, cyano, halo, (C₁-C₃)alkoxy-, (C₁-C₃)alkyl-, hydroxy(C₁-C₆)alkyl-, (C₁-C₃)alkoxy-, (C₁-C₃)alkyl-, CH₃C(O)NH—, CH₃C(O)—, or oxo;
(ii) —(CH₂)ᵣR¹¹, where r is 0, 1 or 2 and R¹¹ is a chemical moiety selected from the group consisting of 1,3-thiazol-4-yl, 1,2,4-oxadiazol-5-yl, 1,2,4-triazol-3-yl, 1,2,5-triazol-3-yl, or 1,3,4-thiadazol-2-yl; wherein said chemical moiety is optionally substituted with 1 to 3 (C₁-C₃)alkyl groups;
(iii) —(CH₂)ₛ—C(OH)(R¹²)(R¹³), where s is 0, 1, or 2 and R¹² and R¹³ are each independently a H or (C₁-C₃)alkyl; and
(iv) —(CH₂)ₜ—C(NH₂)(R¹⁴)(R¹⁵), where t is 0, 1, or 2 and R¹⁴ and R¹⁵ are each independently a H or (C₁-C₃)alkyl;
or a pharmaceutically acceptable salt thereof.

7. The compound of claim 6 wherein
R¹ is hydrogen;
R² is methyl or hydrogen;
m is 0;
R⁹ is
(i) —(CH₂)ₚ—C(O)—N(R^{10a})(R^{10b}), where p is 0, R^{10a} is (C₁-C₆)alkyl- and R^{10b} is —(CH₂)_{q}R^{10c}, where q is 1 and R^{10c} is phenyl, wherein said phenyl is optionally substituted with 1 to 3 substituents each independently selected from halo;
or R^{10a} and R^{10b} taken together with the nitrogen to which they are attached form a 4- to 7-membered heterocycle optionally containing an additional heteroatom selected from oxygen or nitrogen, wherein said heterocycle is optionally substituted with 1 to 3 substituents selected from (C₁-C₃)alkyl-, or hydroxy(C₁-C₆)alkyl-;
(ii) —(CH₂)ᵣ—R¹¹, where r is 1 and R¹¹ is 1,2,4-oxadiazol-5-yl, wherein said 1,2,4-oxadiazol-5-Y1 is optionally substituted with 1 to 3 (C₁-C₃)alkyl groups; or
(iii) —(CH₂)ₛ—C(OH)(R¹²)(R¹³), where s is 1, or 2 and R¹² and R¹³ are each independently a H or (C₁-C₃)alkyl; or
or a pharmaceutically acceptable salt thereof.

8. The compound of claim 1 or 2 having Formula (III) 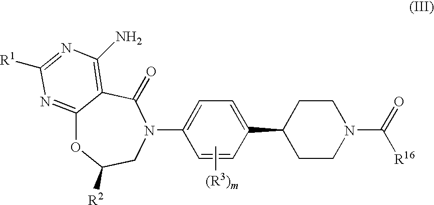
wherein
R¹ is hydrogen, (C₁-C₃)alkyl, methoxy, or halo-substituted (C₁-C₃)alkyl;
R² is hydrogen or methyl;
m is 0, 1 or 2;
R³ is halo, methyl, methoxy, or CF₃, when m is 2, R³ can be the same or different;
R¹⁶ is
(i) —CH(CH₃)—R¹⁷ or —(CH₂)ᵥR¹⁷, where v is 0, 1 or 2 and R¹⁷ is hydrogen, (C₁-C₃)alkyl, (C₁-C₃)alkoxy, (C₁-C₃)alkyl-SO₂—, a 5- to 6-membered cycloalkyl, phenyl, a 5- to 6-membered heterocycle containing 1 to 2 heteroatoms each independently selected from oxygen, nitrogen or sulfur, or a 5- to 6-membered heteroaryl containing 1 to 3 heteroatoms each independently selected from oxygen, nitrogen or sulfur, wherein said alkyl, said cycloalkyl, said phenyl, said heterocycle and said heteroaryl are optionally substituted with 1 to 3 substituents each independently selected from hydroxyl, halo, or (C₁-C₃)alkyl; or
(ii) —(CH₂)_{w}—C(OH)(R¹⁸)(R¹⁹), where w is 0 or 1 and R¹⁸ and R¹⁹ are each independently a H or (C₁-C₃)alkyl;
or a pharmaceutically acceptable thereof.

9. The compound of claim 8 wherein
R¹ is hydrogen;
R² is methyl or hydrogen;
m is 0;
R¹⁶ is —(CH₂)ᵥR¹⁷, where v is 0, 1 or 2 and R¹⁷ is (C₁-C₃)alkyl, a 5- to 6-membered cycloalkyl, phenyl, or a 5- to 6-membered heteroaryl containing 1 to 3 heteroatoms each independently selected from oxygen, or nitrogen;
or a pharmaceutically acceptable thereof.

10. A pharmaceutical composition comprising (i) a compound of any one of the preceding claims; and (ii) a pharmaceutically acceptable excipient, diluent, or carrier.

11. The composition of claim 10 wherein said compound or said pharmaceutically acceptable salt thereof is present in a therapeutically effective amount.

12. The composition of claim 11 further comprising at least one additional pharmaceutical agent selected from the group consisting of an anti-obesity agent and an anti-diabetic agent.

13. The composition of claim 12 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine and said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

14. A method for treating or delaying the progression or onset of Type 2 diabetes and diabetes-related disorders in animals comprising the step of administering to an animal in need of such treatment a therapeutically effective amount of a compound of any one of claims 1 through 9.

15. A method for treating or delaying the progression or onset of Type 2 diabetes and diabetes-related disorders in animals comprising the step of administering to an animal in need of such treatment a pharmaceutical composition of claim 10.

16. A method for treating a disease, condition or disorder modulated by the inhibition of DGAT-1 in animals comprising the step of administering to an animal in need of such treatment two separate pharmaceutical compositions comprising
(iii) first composition comprising a compound of claim 1 through 9, and a pharmaceutically acceptable excipient, diluent, or carrier; and
(iv) a second composition comprising at least one additional pharmaceutical agent selected from the group consisting of an anti-obesity agent and an anti-diabetic agent, and a pharmaceutically acceptable excipient, diluent, or carrier;
wherein said disease, condition or disorder modulated by the inhibition of DGAT-1 is selected from the group consisting of obesity, obesity-related disorders, Type 2 diabetes, and diabetes-related disorders.

17. The method of claim 16 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine; and
said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

18. The method of claim 16 or 17 wherein said first composition and said second composition are administered simultaneously.

19. The method of claim 16 or 17 wherein said first composition and said second composition are administered sequentially and in any order.

20. The use of a compound of claim 1 through 9 or a pharmaceutically acceptable salt thereof in the manufacture of a medicament for treating a disease, condition or disorder that is modulated by the inhibition of DGAT-1.
